(19) United States
(12) Patent Application Publication
(10) Pub. No.: US 2026/0308109 A1
(43) Pub. Date: Oct. 8, 2026

(54) CONFORMATIONALLY RESTRICTED PHENETHYLAMINE ANALOGS

(71) Applicant: 2A Biosciences, Inc., New York, NY (US)

(72) Inventor: David E. Nichols, Chapel Hill, NC (US)

(21) Appl. No.: 19/124,217

(22) PCT Filed: Oct. 24, 2023
(86) PCT No.: PCT/US2023/035834

§ 371 Date: Apr. 24, 2025

Related U.S. Application Data
(60) Provisional application No. 63/418,952, filed on Oct. 24, 2022.

(51) Int. Cl. C07C 217/74; A61K 31/137; A61K 31/36; A61P 27/02; A61P 29/00; C07D 317/70

(52) U.S. Cl. CPC C07C 217/74; A61K 31/137; A61K 31/36; A61P 27/02; A61P 29/00; C07D 317/70

(57) ABSTRACT

The present disclosure relates in some aspects to certain phenethylamine analogs, such as benzocyclobutene and benzocyclopentene analogs, including those of Formula (1). Also provided are methods of synthesizing the compounds, compositions containing the compounds, and methods of using the compounds and compositions. In some aspects, the compounds are useful as therapeutic agents, for example as neuromodulatory agents and as anti-inflammatory agents.

### CROSS-REFERENCE

Priority is claimed under PCT Art. 8(1) and Rule 4.10 to U.S. Prov. App. No. 63/418,952, filed Oct. 24, 2022, and incorporated by reference for all purposes as if fully set forth herein.

### FIELD OF THE INVENTION

The present disclosure relates to conformationally restricted phenethylamine analogs, such as benzocyclobutene and benzocyclopentene analogs. In some aspects, the disclosure further relates to compositions containing the compounds, methods of synthesizing the compounds, and methods of using such compounds, including their administration to subjects. In some aspects, useful features of the compounds include neuromodulatory activity, for example activation of serotonin receptors, inhibition of monoamine transporter uptake, and oral bioavailability. In some further aspects, the compounds are useful as therapeutic agents, such as anti-inflammatory agents.

### BACKGROUND

Psychedelics such as psilocybin and LSD, and entactogens such as MDMA, are currently being investigated for various medical uses, owing to their psychedelic, anxiolytic, and antidepressant effects. Notably, psilocybin has received FDA Breakthrough Therapy designation for its efficacy in treating depression in combination with psychological support. Beyond mental health, psychedelics may be promising for treating inflammatory, neurological, and neurodegenerative diseases and disorders. However, there exists an ongoing need for the development of novel therapeutic compounds that can be used to treat a broad range of disease indications, especially chronic conditions that lack effective treatments. Many psychedelics, including psilocybin and its analogs, remain federally illegal in the United States under Schedule I of the Controlled Substances Act. Novel compounds that minimize side effects, optimize efficacy, and allow for greater access are especially valuable. Provided herein are compounds, compositions, methods, uses, and pharmaceutical kits to meet these needs and others, and having such advantages and improvements as will become readily apparent through the disclosure below.

### INCORPORATION BY REFERENCE

Each cited patent, publication, and non-patent literature is hereby incorporated by reference in its entirety, as if each was incorporated by reference individually, and as if each is fully set forth herein. However, no such citation should be construed as an admission that a cited reference comes from an area that is analogous or directly applicable to the invention, nor should any citation be construed as an admission that a document or underlying information, in any jurisdiction, is prior art or forms part of the common general knowledge in the art.

### BRIEF SUMMARY OF THE INVENTION

The following presents a simplified summary of some embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

In a first aspect, provided is a compound of Formula (1):

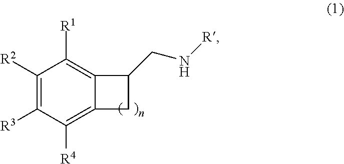

or a pharmaceutically acceptable salt, stereoisomer, hydrate, solvate, or isotopic derivative thereof, wherein: R¹ is H or —OCH₃; R² is H, or is taken together with R³ to form a methylenedioxy group; R³ is H, Br, Cl, I, C₁-C₆ alkyl, C₁-C₆ alkylthio, or C₃-C₆ cycloalkylmethyl; or R³ is taken together with R² to form a methylenedioxy group; R⁴ is H, —OCH₃, or —CH₂CH₂OCH₃; R′ is H or C₁-C₆ alkyl; and n is 1 or 2; and when R³ is H or Br, R⁴ is not —OCH₃; and when R² and R³ are taken together to form a methylenedioxy group, and R¹ and R⁴ are both H, R′ is C₁-C₆ alkyl.

In another aspect, provided is a compound of Formula (1A):

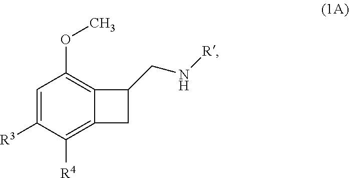

or a pharmaceutically acceptable salt, stereoisomer, hydrate, solvate, or isotopic derivative thereof.

In some embodiments of Formula (1A), R⁴ is —OCH₃. In some embodiments, R⁴ is —CH₂CH₂OCH₃. In some embodiments, R′ is H. In some embodiments, R³ is I.

In some embodiments of Formula (1A), R³ is C₁-C₆ alkyl. In some embodiments, R³ is isobutyl. In some embodiments, R³ is ethyl. In some embodiments, R³ is C₃-C₆ cycloalkylmethyl. In some embodiments, R³ is cyclopropylmethyl.

In some embodiments of Formula (1A), the compound is the R-isomer.

In another aspect, provided is a compound of the formula (1A-I):

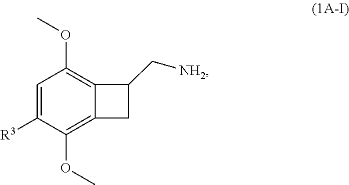

or a pharmaceutically acceptable salt, stereoisomer, hydrate, solvate, or isotopic derivative thereof.

In some embodiments of Formula (1A-I), R³ is I. In some embodiments, R³ is C₁-C₆ alkyl. In some embodiments, R³ is isobutyl. In some embodiments, R³ is ethyl. In some embodiments, the compound is the R-isomer.

In another aspect, provided is the compound of Formula (1B):

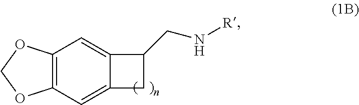

or a pharmaceutically acceptable salt, stereoisomer, hydrate, solvate, or isotopic derivative thereof.

In some embodiments of Formula (1B), n is 1. In some embodiments, n is 2. In some embodiments, R′ is H. In some embodiments, R′ is methyl. In some embodiments, the compound is the R-isomer. In some embodiments, the compound is the S-isomer.

In another aspect, provided is a compound selected from Table 1, or a pharmaceutically acceptable salt, stereoisomer, hydrate, solvate, or isotopic derivative thereof. In some embodiments, a compound selected from Table 1 is the R-isomer.

In another aspect, provided is a compound selected from Table 2, or a pharmaceutically acceptable salt, stereoisomer, hydrate, solvate, or isotopic derivative thereof. In some embodiments, a compound selected from Table 2 is the R-isomer. In some embodiments, a compound selected from Table 2 is the S-isomer.

In another aspect, provided is a compound having the structure of:

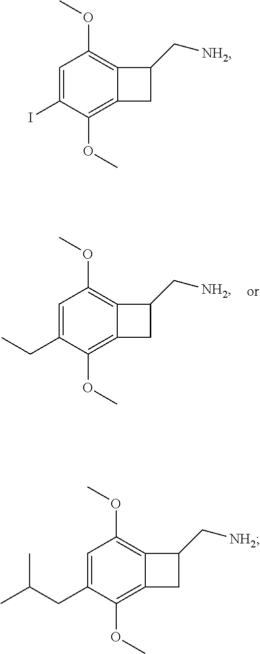 [SMILES: COc1cc(I)c(OC)c2c1C(CN)C2 ; CCc1cc(OC)c2c(c1OC)CC2CN ; COc1cc(CC(C)C)c(OC)c2c1C(CN)C2]

or a pharmaceutically acceptable salt, stereoisomer, hydrate, solvate, or isotopic derivative thereof.

In another aspect, provided is a compound having the structure of:

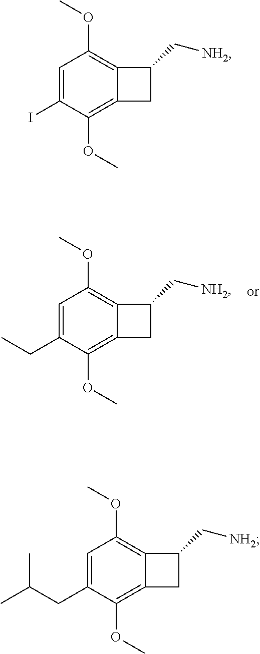 [SMILES: COc1cc(I)c(OC)c2c1[C@H](CN)C2 ; CCc1cc(OC)c2c(c1OC)C[C@H]2CN ; COc1cc(CC(C)C)c(OC)c2c1[C@H](CN)C2]

or a pharmaceutically acceptable salt, hydrate, solvate, or isotopic derivative thereof.

In another aspect, provided is a compound having the structure of:

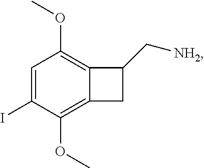

or a pharmaceutically acceptable salt, stereoisomer, hydrate, solvate, or isotopic derivative thereof.

In another aspect, provided is a compound having the structure of:

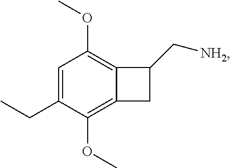 [SMILES: CCc1cc(OC)c2c(c1OC)CC2CN]

or a pharmaceutically acceptable salt, stereoisomer, hydrate, solvate, or isotopic derivative thereof.

In yet another aspect, provided is a compound having the structure of:

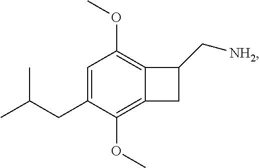 [SMILES: COc1cc(CC(C)C)c(OC)c2c1C(CN)C2]

or a pharmaceutically acceptable salt, stereoisomer, hydrate, solvate, or isotopic derivative thereof.

In another aspect, provided is a compound having the structure of:

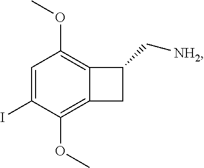

or a pharmaceutically acceptable salt, hydrate, solvate, or isotopic derivative thereof.

In another aspect, provided is a compound having the structure of:

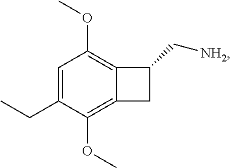 [SMILES: CCc1cc(OC)c2c(c1OC)C[C@H]2CN]

or a pharmaceutically acceptable salt, hydrate, solvate, or isotopic derivative thereof.

In yet another aspect, provided is a compound having the structure of:

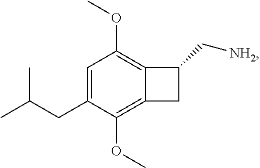 [SMILES: COc1cc(CC(C)C)c(OC)c2c1[C@H](CN)C2]

or a pharmaceutically acceptable salt, hydrate, solvate, or isotopic derivative thereof.

In yet another aspect, provided is a pharmaceutical composition comprising a therapeutically effective amount of the compound of any of the preceding embodiments, or a pharmaceutically acceptable salt, prodrug, hydrate, or solvate thereof, and a pharmaceutically acceptable carrier, diluent, or excipient.

In some embodiments (equivalently herein, and simply for shorthand, “in embodiments”), the pharmaceutical composition is suitable for oral, buccal, sublingual, intranasal, injectable, subcutaneous, intravenous, intraocular, topical, or transdermal administration.

In some embodiments, the pharmaceutical composition is provided in unit dosage form. In some such embodiments, the compound is in a total amount of between 0.01 and 100 mg.

In some embodiments, the composition further comprises a therapeutically effective amount of an additional active compound, or a pharmaceutically acceptable salt, solvate, or stereoisomer thereof. In some embodiments, the additional active compound is selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, dissociatives, cannabinoids, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, plasticity-inducing agents (e.g., psychoplastogens), monoamine oxidase inhibitors, tryptamines, terpenes, phenethylamines, sedatives, stimulants, serotonergic agents, NMDA modulators, NMDA antagonists, and vitamins.

In embodiments, the pharmaceutical composition comprises the R isomer of a disclosed compound or a pharmaceutically acceptable salt, hydrate, solvate, or isotopic derivative thereof, in an enantiomeric excess of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

In embodiments, the pharmaceutical composition comprises the S isomer of a disclosed compound, or a pharmaceutically acceptable salt, hydrate, solvate, or isotopic derivative thereof, in an enantiomeric excess of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

In another aspect, provided is a method for modulating neurotransmission in a subject, comprising administering to the subject a disclosed compound or composition. In some embodiments, modulating neurotransmission comprises agonizing the 5-HT_{2A} or 5-HT_{2C} receptor.

In some aspects are provided methods of increasing neural plasticity in a subject, comprising administering to the subject a disclosed compound or composition of any one of the preceding embodiments.

In another aspect, provided is a method of treating a medical condition in a subject in need of such treatment, comprising administering to the subject a disclosed compound or composition of any one of the preceding embodiments.

In some embodiments, the medical condition is a disorder linked to dysregulation or inadequate functioning of serotonergic neurotransmission.

In some embodiments, the medical condition is a mental, behavioral, or neurodevelopmental disorder. In some embodiments, the medical condition is a neurodevelopmental disorder, schizophrenia or another primary psychotic disorder, catatonia, a mood disorder, an anxiety or fear-related disorders, an obsessive-compulsive or related disorder, a disorder specifically associated with stress, a dissociative disorder, a feeding or eating disorder, an elimination disorder, a disorder of bodily distress or bodily experience, a disorder due to substance use or addictive behavior, an impulse control disorder, a disruptive behavior or dissocial disorder, a personality disorder, a paraphilic disorder, a factitious disorder, a neurocognitive disorder, a mental or behavioral disorder associated with pregnancy, childbirth or the puerperium, a sleep-wake disorder, or a sexual dysfunction.

In some embodiments, the compound or composition is administered together with one or more sessions of psychotherapy.

In some embodiments, the medical condition is inflammation or an inflammatory disorder. In some embodiments, the medical condition is skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, ocular inflammation, or brain inflammation. In some embodiments, the inflammatory disorder is an acute inflammatory disorder. In some embodiments, the inflammatory disorder is a chronic inflammatory disorder. In some embodiments, the inflammatory disorder is a steroid-resistant disorder.

In some embodiments, the inflammatory disorder is selected from the group consisting of asthma, chronic obstructive pulmonary disease, neuroinflammation, rheumatoid arthritis, atherosclerosis, psoriasis, type II diabetes, inflammatory bowel disease, Crohn's disease, multiple sclerosis, septicemia, conjunctivitis, Alzheimer's disease.

In some embodiments, the inflammatory disorder is dermatitis. In some embodiments, the inflammatory disorder is atopic dermatitis, chronic photosensitivity dermatitis, eczema, atopic eczema, contact eczema, dryness eczema, seborrheic eczema, discoid eczema, varicose eczema, herpetic dermatitis, neurodermatitis, autosensitizing dermatitis, stasis dermatitis, purulent dermatitis, dyshidrotic eczema, follicular eczema, spongiotic dermatitis, hand dermatitis, diaper dermatitis, occupational contact dermatitis, and lichen planus-like atopic dermatitis.

In some embodiments, the subject has a compromised immune system. In some embodiments, the subject has an autoimmune disorder. In some embodiments, the subject has a contraindication to a corticosteroid.

In some embodiments, treating inflammation or an inflammatory disorder comprises reducing the level of an inflammatory biomarker by about 1%, 5%, 10%, 20%, 30%, 40%, 50%, 60%, 70%, 80%, 90%, 95%, or 100%. In some embodiments, the inflammatory biomarker is an inflammatory response gene product. In some embodiments, the inflammatory response gene product is mRNA. In some embodiments, the mRNA is ICAM1, VCAM1, MCP1, IL-6, IL-1B, Gm-csf, IL-5, IL-9, IL-15, Muchac, mmp9, or TGF-β mRNA. In some embodiments, the inflammatory response gene product is a protein. In some embodiments, the protein is ICAM1, VCAM1, MCP1, IL-6, IL-1B, Gm-csf, IL-5, IL-9, IL-15, Muc5ac, mmp9, or TGF-β.

In some embodiments, the medical condition is an ophthalmic disorder. In some embodiments, the ophthalmic disorder is an inflammatory disorder. In some embodiments, the ophthalmic disorder is macular degeneration, keratoconjunctivitis, conjunctivitis, keratitis, diabetic retinopathy, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, or retinochoroidal neovascularization.

In some embodiments, the medical condition is a neurodegenerative disorder. In some embodiments, the neurodegenerative disorder is selected from the group consisting of Alzheimer's disease, amyotrophic lateral sclerosis or Charcot's disease, chronic traumatic encephalopathy, corticobasal degeneration, dementias including vascular dementia, Huntington's disease, Lytico-Bodig disease, mild cognitive impairment, multiple sclerosis, a motor neuron disease, neuromyelitis optica spectrum disorder, Parkinson's disease or Parkinsonisms, prion diseases, progressive supranuclear palsy, and traumatic brain injury.

In another aspect, provided is a disclosed compound or composition for use in the treatment of a medical condition.

In another aspect, provided is a disclosed compound or composition for the manufacture of a medicament for the treatment of a medical condition.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the invention as set forth in the claims.

The headings within this document are being utilized only to expedite its review by a reader. They should not be construed as limiting the invention in any manner.

### BRIEF DESCRIPTION OF THE FIGURES

To further clarify various aspects of the invention, a more particular description is rendered by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. These figures, and the elements depicted therein, are not necessarily drawn to consistent scale or to any scale. Unless context suggests otherwise, like elements are indicated by like numerals. Certain aspects of the invention are therefore further described and explained with additional specificity and detail, but still by way of example only, with reference to the accompanying figures in which:

FIG. 1 shows an exemplary 1H nuclear magnetic resonance (NMR) spectrum of 3-iodo-2,5-dimethoxybicyclo[4.2.0]octa-1,3,5-trien-7-yl)methanamine (Compound 3) acquired in DMSO-d₆.

FIG. 2 shows an exemplary liquid chromatography (LC) chromatogram obtained from a sample of Compound 2. (a) λ=220 nm; (b) λ=254 nm; (c) Total Ion Chromatogram.

FIG. 3 shows an exemplary mass spectrum obtained from a sample of Compound 2.

FIG. 4 shows an exemplary ¹H NMR spectrum of Compound 2 acquired in DMSO-d₆, including annotated peak assignments.

FIG. 5 shows an exemplary Heteronuclear Single Quantum Correlation-Distortionless Enhancement by Polarization Transfer (HSQC-DEPT) NMR spectrum of Compound 2 acquired in DMSO-d₆, including annotated peak assignments.

FIG. 6 shows an exemplary Heteronuclear Multiple Bond Correlation (HMBC) NMR spectrum of Compound 2 acquired in DMSO-d₆, including annotations showing 1H-13C correlations.

FIG. 7 shows an exemplary Nuclear Overhauser Effect Spectroscopy (NOESY) NMR spectrum of Compound 2 acquired in DMSO-d₆, including annotations showing through-space ¹H-¹H correlations.

FIG. 8 shows an exemplary ¹H NMR spectrum of (3-ethyl-2,5-dimethoxybicyclo [4.2.0]octa-1,3,5-trien-7-yl)methanamine (Compound 4) acquired in DMSO-d₆.

FIG. 9 shows an exemplary LC chromatogram obtained from a sample of Compound 4. (a) λ=220 nm; (b) λ=254 nm; (c) Total Ion Chromatogram.

FIG. 10 shows an exemplary mass spectrum obtained from a sample of Compound 4.

FIG. 11 shows an exemplary ¹H NMR spectrum of (3-isobutyl-2,5-dimethoxybicyclo [4.2.0]octa-1,3,5-trien-7-yl)methanamine (Compound 8) acquired in DMSO-d₆.

FIG. 12 shows an exemplary LC chromatogram obtained from a sample of Compound 8. (a) λ=220 nm; (b) λ=254 nm; (c) Total Ion Chromatogram.

FIG. 13 shows an exemplary mass spectrum obtained from a sample of Compound 8.

FIG. 14 shows cell based agonist IP1 assay data for Compound 2, as described in Example 15.

FIG. 15 shows cell based agonist IP1 assay data for Compound 4, as described in Example 15.

FIG. 16 shows cell based agonist IP1 assay data for Compound 8, as described in Example 15.

### DETAILED DESCRIPTION OF THE INVENTION

While various aspects and features of certain embodiments are summarized above, the following detailed description illustrates several exemplary embodiments in further detail to enable one of skill in the art to practice such embodiments, and to make and use the full scope of the invention claimed. The described examples are provided for illustrative purposes and are not intended to limit the scope of the invention or its applications. It will be understood that many modifications, substitutions, changes, and variations in the described examples, embodiments, applications, and details of the invention illustrated herein can be made by those skilled in the art without departing from the spirit of the invention, or the scope of the invention as described in the appended claims. It also will be appreciated that the headings within this document are being utilized only to expedite its review by a reader. They should not be construed as limiting the invention in any manner.

The scope of the invention includes all embodiments and formulations thereof, not only those expressly described below, and it will be understood that many modifications, substitutions, changes, and variations in the described embodiments, applications, and details of the invention illustrated herein can be made by those skilled in the art without departing from the spirit of the invention, or the scope of the invention as set forth in the appended claims.

### A. General Definitions and Terms

As used in this specification and the appended claims, the singular forms “a,” “an,” and “the” include plural referents unless the context clearly dictates otherwise. Thus, for example, reference to “an active agent” includes reference to a combination of two or more active agents, and reference to “an excipient” includes reference to a combination of two or more excipients. While the term “one or more” may be used, its absence (or its replacement by the singular) does not signify the singular only, but simply underscores the possibility of multiple agents or ingredients in particular embodiments. The terms “comprising,” “including,” “such as,” and “having” are inclusive and not exclusive (i.e., there may be other elements in addition to the recited elements). The term “or” is used herein to mean, and is used interchangeably with, the term “and/or,” unless context clearly indicates otherwise.

Unless otherwise indicated, all numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, “about” refers to plus or minus five percent (+5%) of the recited unit of measure. The term “substantially,” where it is applied to modify a feature or limitation herein, will be read in the context of the invention and in light of the knowledge in the art to provide the appropriate certainty, e.g., by using a standard that is recognized in the art for measuring the meaning of “substantially” as a term of degree, or by ascertaining the scope as would one of skill in the art.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments may contain certain errors necessarily resulting from the standard deviation found in their respective testing measurements.

A comprehensive list of the abbreviations utilized by organic chemists of ordinary skill in the art appears in the first issue of each volume of the Journal of Organic Chemistry; this list is typically presented in a table entitled Standard List of Abbreviations; the current list as of the date of this filing is hereby incorporated by reference as if fully set forth herein.

Unless defined otherwise, all technical and scientific terms herein have the meaning as commonly understood by one having ordinary skill in the art to which this invention belongs, who as a shorthand may be referred to simply as “one of skill.” Further definitions that may assist a reader in understanding the disclosed embodiments follow; however, it will be appreciated that such definitions are not intended to limit the scope of the invention, which shall be properly interpreted and understood by reference to the full specification (as well as any plain meaning known to one of skill) in view of the language used in the claims. Terminology used herein is for the purpose of describing particular embodiments only and is not intended to be limiting.

Generally, the nomenclature used and procedures performed herein are those known in fields relating to one or more aspects of the invention, such as biology, pharmacology, neuroscience, organic chemistry, synthetic chemistry, and/or medicinal chemistry, and are those that will be well known and commonly employed in such fields. Standard techniques and procedures will be those generally performed according to conventional methods in the art.

“Alkyl” will be understood to include straight or branched radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” can also be used. Preferably, an alkyl group comprises from 1 to 10 carbon atoms, more preferably from 1 to 6 carbon atoms, more preferably from 1 to 4 carbon atoms, and most preferably from 1 to 3 carbon atoms. For any alkyl, the alkyl may be optionally substituted at one or more positions by deuterium, halogen, alkyl, alkenyl, alkynyl, alkyl ester, hydroxy, alkoxy, carboxy, formyl, aryl, cycloalkyl, cycloalkenyl, heterocycloalkyl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, —OP(O)(OH)₂, —OC(O)H, —OSO₂OH, —OC(O)NH₂, and —SONH₂. In embodiments, an alkyl group will be optionally substituted. In embodiments, an alkyl group will be substituted at one or more positions. In embodiments, an alkyl group will not be substituted at any positions.

“Alkenyl” refers to an unsaturated branched, straight-chain, or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include ethenyl; propenyls such as prop-1-en-1-yl, prop-1-en-2-yl, prop-2-en-1-yl (allyl), prop-2-en-2-yl, cycloprop-1-en-1-yl, and cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2yl, buta-1,3-dien-1-yl, buta-1,3-dien-2-yl, cyclobut-1-en-1-yl, cyclobut-1-en-3-yl, and cyclobuta-1,3-dien-1-yl; and the like. An alkenyl group can be substituted or unsubstituted.

“Alkynyl” refers to an unsaturated branched, straight-chain, or cyclic alkyl radical having at least one carbon-carbon triple bond derived by the removal of one hydrogen atom from a single carbon atom of a parent alkyne. Typical alkynyl groups include ethynyl; propynyls such as prop-1-yn-1-yl, and prop-2-yn-1-yl; butynyls such as but-1-yn-1-yl, but-1-yn-3-yl, and but-3-yn-1-yl; and the like. An alkynyl group can be substituted or unsubstituted.

“Aryl” refers to a monovalent aromatic hydrocarbon radical derived by the removal of one hydrogen atom from a single carbon atom of a parent aromatic ring system. Typical aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. Preferably, an aryl group comprises from 6 to 20 carbon atoms, more preferably, between 6 to 12 carbon atoms. An aryl group can be substituted or unsubstituted.

“Cycloalkyl” refers to a saturated monocyclic, bicyclic, fused bicyclic or bridged polycyclic ring assembly containing from 3 to 12 ring atoms, or the number of atoms indicated. Cycloalkyl can include any number of carbons, such as 3 to 6 carbon atoms, 4 to 6 carbon atoms, 5 to 6 carbon atoms, 3 to 8 carbon atoms, 4 to 8 carbon atoms, 5 to 8 carbon atoms, 6 to 8 carbon atoms, 7 to 8 carbon atoms, 3 to 9 carbon atoms, 4 to 9 carbon atoms, 5 to 9 carbon atoms, 6 to 9 carbon atoms, 7 to 9 carbon atoms, 8 to 9 carbon atoms, 3 to 10 carbon atoms, 4 to 10 carbon atoms, 5 to 10 carbon atoms, 6 to 10 carbon atoms, 7 to 10 carbon atoms, 8 to 10 carbon atoms, 9 to 10 carbon atoms, 3 to 11 carbon atoms, 4 to 11 carbon atoms, 5 to 11 carbon atoms, 6 to 11 carbon atoms, 7 to 11 carbon atoms, 8 to 11 carbon atoms, 9 to 11 carbon atoms, 10 to 11 carbon atoms, 3 to 12 carbon atoms, 4 to 12 carbon atoms, 5 to 12 carbon atoms, 6 to 12 carbon atoms, 7 to 12 carbon atoms, 8 to 12 carbon atoms, 9 to 12 carbon atoms, 10 to 12 carbon atoms, and 11 to 12 carbon atoms. Monocyclic cycloalkyl rings include, for example, cyclopropyl, cyclobutyl, cyclopentyl, cyclohexyl, and cyclooctyl. Bicyclic compounds include spirocyclic compounds, fused bicyclic compounds and bridged bicyclic compounds. Bicyclic and polycyclic cycloalkyl rings include, for example, norbornane, bicyclooctane, decahydronaphthalene and adamantane. When cycloalkyl is a monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a monocyclic C₃₋₆ cycloalkyl, exemplary groups include, but are not limited to cyclopropyl, cyclobutyl, cyclopentyl, and cyclohexyl. A cycloalkyl group can be substituted or unsubstituted.

“Cycloalkenyl” refers to a mono- or multi-cyclic hydrocarbon ring system that contains one or more double bonds in at least one ring. However, if there is more than one double bond, the double bonds cannot form a fully delocalized pi-electron system throughout all the rings (otherwise the group would be “aryl,” as defined herein). When composed of two or more rings, the rings may be connected together in a fused fashion. Cycloalkenyl can include any number of carbons, such as 3 to 6 carbon atoms, 4 to 6 carbon atoms, 5 to 6 carbon atoms, 3 to 8 carbon atoms, 4 to 8 carbon atoms, 5 to 8 carbon atoms, 6 to 8 carbon atoms, 7 to 8 carbon atoms, 3 to 9 carbon atoms, 4 to 9 carbon atoms, 5 to 9 carbon atoms, 6 to 9 carbon atoms, 7 to 9 carbon atoms, 8 to 9 carbon atoms, 3 to 10 carbon atoms, 4 to 10 carbon atoms, 5 to 10 carbon atoms, 6 to 10 carbon atoms, 7 to 10 carbon atoms, 8 to 10 carbon atoms, 9 to 10 carbon atoms, 3 to 11 carbon atoms, 4 to 11 carbon atoms, 5 to 11 carbon atoms, 6 to 11 carbon atoms, 7 to 11 carbon atoms, 8 to 11 carbon atoms, 9 to 11 carbon atoms, 10 to 11 carbon atoms, 3 to 12 carbon atoms, 4 to 12 carbon atoms, 5 to 12 carbon atoms, 6 to 12 carbon atoms, 7 to 12 carbon atoms, 8 to 12 carbon atoms, 9 to 12 carbon atoms, 10 to 12 carbon atoms, and 11 to 12 carbon atoms. Representative Cycloalkenyl groups include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. A cycloalkenyl group may be unsubstituted or substituted.

“Cycloalkylmethyl” refers to a radical having a methylene component and a cycloalkyl component, where the methylene component links the cycloalkyl component to the point of attachment. The cycloalkyl component is as defined above, and can include any number of carbons, such as 3 to 6 carbon atoms (i.e., a C₃-C₆ cycloalkylmethyl), 4 to 6 carbon atoms, 5 to 6 carbon atoms, 3 to 8 carbon atoms, 4 to 8 carbon atoms, 5 to 8 carbon atoms, 6 to 8 carbon atoms, 7 to 8 carbon atoms, 3 to 9 carbon atoms, 4 to 9 carbon atoms, 5 to 9 carbon atoms, 6 to 9 carbon atoms, 7 to 9 carbon atoms, 8 to 9 carbon atoms, 3 to 10 carbon atoms, 4 to 10 carbon atoms, 5 to 10 carbon atoms, 6 to 10 carbon atoms, 7 to 10 carbon atoms, 8 to 10 carbon atoms, 9 to 10 carbon atoms, 3 to 11 carbon atoms, 4 to 11 carbon atoms, 5 to 11 carbon atoms, 6 to 11 carbon atoms, 7 to 11 carbon atoms, 8 to 11 carbon atoms, 9 to 11 carbon atoms, 10 to 11 carbon atoms, 3 to 12 carbon atoms, 4 to 12 carbon atoms, 5 to 12 carbon atoms, 6 to 12 carbon atoms, 7 to 12 carbon atoms, 8 to 12 carbon atoms, 9 to 12 carbon atoms, 10 to 12 carbon atoms, and 11 to 12 carbon atoms. In some preferred embodiments, the cycloalkylmethyl group is a cyclopropylmethyl group. A cycloalkylmethyl group can be substituted or unsubstituted.

“Halogen” refers to fluorine, chlorine, bromine, and iodine.

“Heterocycloalkyl” refers to a cycloalkyl as defined above, having from 3 to 12 ring members and from 1 to 4 heteroatoms of N, O and S. Heterocycloalkyl includes bicyclic compounds which include a heteroatom. Bicyclic compounds includes spirocyclic compounds, fused bicyclic compounds, and bridged bicyclic compounds The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C1-6 alkyl or oxo (═O), among many others.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C0-6, C1-2, C1-3, C1-4, C1-5, C1-6, C2-3, C2-4, C2-5, C2-6, C3-4, C3-5, C3-6, C4-5, C4-6 and C5-6. In some instances, the alkyl component can be absent. The heterocycloalkyl component is as defined above. Alkyl-heterocycloalkyl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. A heteroaryl includes groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C0-6, C1-2, C1-3, C1-4, C1-5, C1-6, C2-3, C2-4, C2-5, C2-6, C3-4, C3-5, C3-6, C4-5, C4-6 and C5-6. In some instances, the alkyl component can be absent. The heteroaryl component is as defined within. Alkyl-heteroaryl groups can be substituted or unsubstituted.

“Alkoxy” refers to the formula-OR, wherein R is an alkyl, an alkenyl, an alkynyl, a cycloalkyl, a cycloalkenyl, aryl, or heterocyclyl, as defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

“Alkylthio” or “thioalkyl” refers to the formula-SR, wherein R is an alkyl, an alkenyl, an alkynyl, a cycloalkyl, a cycloalkenyl, aryl, or heterocyclyl, as defined herein. A non-limiting list of alkylthio are methylthio, ethylthio, n-propylthio, isopropylthio, n-butylthio, iso-butylthio, sec-butylthio, phenylthio, and benzylthio. An alkylthio may be substituted or unsubstituted.

“Acyl” refers to a hydrogen, an alkyl, an alkenyl, an alkynyl, a cycloalkyl, a cycloalkenyl, aryl, or heterocyclyl, connected via a carbonyl group as a substituent. Examples include formyl, acetyl, propanoyl, benzoyl, and acryl. An acyl may be substituted or unsubstituted.

“Haloalkyl” refers to any alkyl group as defined above, wherein one or more hydrogen atoms are replaced by a halogen (e.g., a fluorine, a chlorine, a bromine, or an iodine). Where an alkyl radical is substituted by more than one halogen, it may be referred to using a prefix corresponding to the number of halogen substitutions. For example, dihaloalkyl refers to an alkyl substituted by two halo groups, which may be, but are not necessarily, the same halogen. Examples of haloalkyl groups include difluoromethyl (—CHF₂), bromofluoromethyl (—CHBrF), trifluoromethyl (—CF₃), and 2-fluoroethyl (—CH₂CH₂F). Additional examples of haloalkyl groups include —CHF₂, —CH₂F, —CH₂CF₃, —CH₂CHF₂, —CH₂CH₂F, —CH(CH₃)(CF₃), CH(CH₃)(CHF₂), and —CH(CH₃)(CH₂F). A haloalkyl may be substituted or unsubstituted.

“Hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

“Haloalkoxy” refers to an —O-alkyl group in which one or more of the hydrogen atoms are replaced by a halogen (e.g., mono-haloalkoxy, di-haloalkoxy and tri-haloalkoxy). The halogens may be the same or different in each instance. Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

“Sulfenyl” refers to an —SR group in which R can be hydrogen, alkyl, alkenyl, alkynyl, cycloalkyl, cycloalkenyl, aryl, or heterocyclyl, as defined herein. A sulfenyl may be substituted or unsubstituted.

“Sulfinyl” refers to an —S(═O)—R group in which R can be the same as defined with respect to sulfenyl. A sulfinyl may be substituted or unsubstituted.

“Sulfonyl” refers to an —SO₂R group in which R can be the same as defined with respect to sulfenyl. A sulfonyl may be substituted or unsubstituted.

“O-carboxy” refers to a —RC(═O)O— group in which R can be hydrogen, alkyl, alkenyl, alkynyl, cycloalkyl, cycloalkenyl, aryl, or heterocyclyl, as defined herein. An O-carboxy may be substituted or unsubstituted.

“Ester” and “C-carboxy” refer to a —C(═O) OR group in which R can be the same as defined with respect to O-carboxy. Ester and C-carboxy groups may be substituted or unsubstituted.

“Thiocarbonyl” refers to a —C(═S) R group in which R can be the same as defined with respect to O-carboxy. A thiocarbonyl may be substituted or unsubstituted.

“Trihalomethanesulfonyl” refers to an X₃CSO₂— group wherein each X is a halogen.

“Trihalomethanesulfonamido” refers to an X₃CS(O)₂N(R_{A})— group wherein each X is a halogen, and R^{A} is hydrogen, alkyl, alkenyl, alkynyl, cycloalkyl, cycloalkenyl, aryl, or heterocyclyl, as defined herein.

“S-sulfonamido” refers to a —SO₂N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An S-sulfonamido may be substituted or unsubstituted.

“N-sulfonamido” refers to a RSO₂N(R_{A})— group in which R and R_{A} can be independently hydrogen, alkyl, alkenyl, alkynyl, cycloalkyl, cycloalkenyl, aryl, or heterocyclyl, as defined herein. An N-sulfonamido may be substituted or unsubstituted.

“O-carbamyl” refers to a —OC(═O)N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An O-carbamyl may be substituted or unsubstituted.

“N-carbamyl” refers to an ROC(═O)N(R_{A})— group in which R and R_{A} can be independently hydrogen, alkyl, alkenyl, alkynyl, cycloalkyl, cycloalkenyl, aryl, or heterocyclyl, as defined herein. An N-carbamyl may be substituted or unsubstituted.

“O-thiocarbamyl” refers to a —OC(═S)—N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An O-thiocarbamyl may be substituted or unsubstituted.

“N-thiocarbamyl” refers to an ROC(═S)N(R_{A})— group in which R and R_{A} can be independently hydrogen, alkyl, alkenyl, alkynyl, cycloalkyl, cycloalkenyl, aryl, or heterocyclyl, as defined herein. An N-thiocarbamyl may be substituted or unsubstituted.

“C-amido” group refers to a —C(═O)N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. A C-amido may be substituted or unsubstituted.

“N-amido” refers to a RC(═O)N(R_{A})— group in which R and R_{A} can be independently hydrogen, alkyl, alkenyl, alkynyl, cycloalkyl, cycloalkenyl, aryl, or heterocyclyl, as defined herein. An N-amido may be substituted or unsubstituted.

“Optionally substituted” unless otherwise specified means that a group may be unsubstituted, or substituted by one or more of the substituents listed for that group. Likewise, when a group is described as being “unsubstituted or substituted” if substituted, the substituent(s) may be selected from one or more of the indicated substituents. When there are more than one substituents, the substituents may be the same or different. In one embodiment, an optionally substituted group has one substituent. In another embodiment, an optionally substituted group has two substituents. In another embodiment, an optionally substituted group has three substituents. In another embodiment, an optionally substituted group has four substituents. If no substituents are indicated for an “optionally substituted” or “substituted” group, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amino group, a di-substituted amino group, and a tri-substituted amino group.

Still additional definitions and abbreviations are provided elsewhere herein.

### B. Compounds

The phenethylamine pharmacophore is one of the most well-known chemical scaffolds found in biologically active molecules, such as neurotransmitters (e.g., dopamine) and psychoactive drugs (e.g., the entactogen 3,4-methylenedioxymethamphetamine, also known as MDMA).

One class of compounds, known as the “2C” or “2C-X” compounds, are ring-substituted phenethylamines containing methoxy groups on the 2 and 5 positions of the benzene ring, and an additional (often lipophilic) substituent at the 4 position (i.e., R″ below).

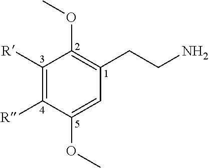

Certain 2C compounds may have effects that are similar to those of entactogens such as MDMA, as well as effects that are similar to those of psychedelics such as psilocybin. Although some 2C compounds are generally well-tolerated within certain dose ranges, adverse sympathomimetic effects have been reported, including agitation, excited delirium, aggression, violence, dysphoria, hypertension, tachycardia, seizures, and hyperthermia, and many 2C compounds are known to be generally associated with heavy “body load” and gastrointestinal effects (see, e.g., Dean et al., J Med Toxicol, 2013; 9(2):172-178).

A small number of conformationally restricted phenethylamine analogs, such as 3-bromo-3,6-dimethoxybenzocyclobuten-1-yl)methylamine and 4-bromo-3,6-dimethoxybenzocyclobuten-1-yl)methylamine have been reported in the literature (McLean et al., J. Med. Chem. 2006; 49:5794-5803; McLean et al. J. Med. Chem. 2006, 49, 14, 4269-4274). The compound 4-bromo-3,6-dimethoxybenzocyclobuten-1-yl)methylamine was found to be a potent and selective 5-HT_{2A} receptor agonist. Reports of other conformationally restricted phenethylamines are scarce in the scientific literature, and to date, no phenethylamines based on a benzocycloalkene core structure have been employed in therapeutic applications.

The present disclosure relates to conformationally restricted phenethylamine analogs, such as benzocyclobutene and benzocyclopentene analogs. In some aspects, the disclosure further relates to methods of synthesizing the compounds, compositions containing the compounds, and methods of using such compounds, including their administration to subjects. In some aspects, useful features of the compounds include neuromodulatory activity, for example, activation of serotonin receptors, inhibition of monoamine transporter uptake, and oral bioavailability.

In one aspect, provided is a compound of Formula (1):

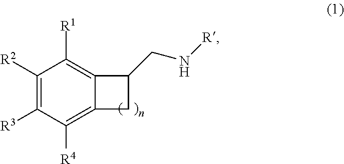

or a pharmaceutically acceptable salt, stereoisomer, hydrate, solvate, or isotopic derivative thereof, wherein R¹ is H or —OCH₃; R² is H, or is taken together with R³ to form a methylenedioxy group; R³ is H, Br, Cl, I, C₁-C₆ alkyl, C₁-C₆ alkylthio, or C₃-C₆ cycloalkylmethyl; or R³ is taken together with R² to form a methylenedioxy group; R⁴ is H, —OCH₃, or —CH₂CH₂OCH₃; R′ is H or C₁-C₆ alkyl; and n is 1 or 2.

In some embodiments, R¹ is H or —OCH₃. In some embodiments, R¹ is H. In other embodiments, R¹ is —OCH₃.

In some embodiments, R² is H, or is taken together with R³ to form a methylenedioxy group. In some embodiments, R² is H. In other embodiments, R² is taken together with R³ to form a methylenedioxy group.

In some embodiments, R³ is H, Br, Cl, I, C₁-C₆ alkyl, C₁-C₆ alkylthio, or C₃-C₆ cycloalkylmethyl. In some embodiments, R³ is H. In other embodiments, R³ is halogen (e.g., R³ is Br, Cl, or I). In some preferred embodiments, R³ is Cl, I, C₁-C₆ alkyl, C₁-C₆ alkylthio, or C₃-C₆ cycloalkylmethyl. In some embodiments, R³ is Br. In some embodiments, R³ is Cl or I. In some embodiments, R³ is Cl. In some embodiments, R³ is I. In some embodiments, R³ is C₁-C₆ alkyl. In some such embodiments, R³ is butyl (e.g., R³ is n-butyl, isobutyl, sec-butyl, or tert-butyl). In some embodiments, R³ is isobutyl. In some embodiments, R³ is C₁-C₆ alkylthio. In some embodiments, R³ is C₃-C₆ cycloalkyl (e.g., R³ is cyclopropyl, cyclobutyl, cyclopentyl, or cyclohexyl). In some embodiments, R³ is cyclopropyl. In other embodiments, R³ is taken together with R² to form a methylenedioxy group.

In certain embodiments, wherein R² and R³ are taken together to form a methylenedioxy group, it will be understood that “a methylenedioxy group” refers to a moiety having the formula

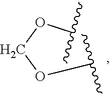 [SMILES: *OCO*]

wherein the wavy lines indicate the points of connectivity to the remainder of the compound. In some embodiments, R² and R³ are taken together to form a methylenedioxy group, and R¹ is —OCH₃. In some embodiments, R² and R³ are taken together to form a methylenedioxy group, and R⁴ is —OCH₃. In some embodiments, R² and R³ are taken together to form a methylenedioxy group, and R′ is H. In some embodiments, R² and R³ are taken together to form a methylenedioxy group, and R′ is C₁-C₆ alkyl. In some embodiments, R² and R³ are taken together to form a methylenedioxy group, and R′ is methyl.

In some embodiments, R⁴ is H. In some embodiments, R⁴ is H or —OCH₃. In some embodiments, R⁴ is H or —CH₂CH₂OCH₃. In some embodiments, R⁴ is not H. In some embodiments, R⁴ is —OCH₃ or —CH₂CH₂OCH₃. In some embodiments, R⁴ is —OCH₃. In other embodiments, R⁴ is —CH₂CH₂OCH₃.

In some embodiments, R′ is H or C₁-C₆ alkyl. In some embodiments, R′ is H. In some embodiments, R′ is C₁-C₆ alkyl.

In some embodiments, n is 1 or 2. In some embodiments, n is 1. In embodiments, n is 2.

In some embodiments, R¹ and R⁴ are both-OCH₃. In some embodiments, R¹ and R⁴ are both-OCH₃, and R³ is I. In some embodiments, R¹ and R⁴ are both-OCH₃, R³ is I, and n is 1. In some embodiments, R¹ and R⁴ are both-OCH₃, and R³ is C₁-C₆ alkyl. In some embodiments, R¹ and R⁴ are both-OCH₃, and R³ is ethyl. In some embodiments, R¹ and R⁴ are both-OCH₃, R³ is ethyl, and n is 1. In some embodiments, R¹ and R⁴ are both-OCH₃, and R³ is isobutyl. In some embodiments, R¹ and R⁴ are both-OCH₃, R³ is isobutyl, and n is 1.

In some embodiments of Formula (1) wherein R³ is Br, R⁴ is other than —OCH₃. In some embodiments wherein R³ is Br, R⁴ is not —OCH₃. In some embodiments wherein R³ is Br, R⁴ is H or —CH₂CH₂OCH₃. In some embodiments wherein R³ is Br, R⁴ is H. In some embodiments wherein R³ is Br, R⁴ is —CH₂CH₂OCH₃. In some embodiments wherein R³ is Br, R¹ and R⁴ are not both-OCH₃. In some embodiments wherein R³ is Br, R′ is not H. In some embodiments wherein R³ is Br, R′ is C₁-C₆ alkyl. In some embodiments, R³ is not Br.

In some embodiments of Formula (1) wherein R³ is H, R⁴ is other than —OCH₃. In some embodiments wherein R³ is H, R⁴ is not —OCH₃. In some embodiments wherein R³ is H, R⁴ is H or —CH₂CH₂OCH₃. In some embodiments wherein R³ is H, R⁴ is H. In some embodiments wherein R³ is H, R⁴ is —CH₂CH₂OCH₃. In some embodiments wherein R³ is H, R¹ and R⁴ are not both —OCH₃. In some embodiments wherein R³ is H, R′ is not H. In some embodiments wherein R³ is H, R′ is C₁-C₆ alkyl. In some embodiments wherein R³ is H, n is 2. In embodiments, R³ is not H.

In some embodiments of Formula (1) wherein R³ is H or Br, R⁴ is other than —OCH₃. In some embodiments wherein R³ is H or Br, R⁴ is not —OCH₃. In some embodiments wherein R³ is H or Br, R⁴ is H or —CH₂CH₂OCH₃. In some embodiments wherein R³ is H or Br, R⁴ is H. In some embodiments wherein R³ is H or Br, R⁴ is —CH₂CH₂OCH₃. In some embodiments wherein R³ is H or Br, R¹ and R⁴ are not both-OCH₃. In some embodiments wherein R³ is H or Br, R′ is not H. In some embodiments wherein R³ is H or Br, R′ is C₁-C₆ alkyl. In some embodiments wherein R³ is H or Br, n is 2. In embodiments, R³ is not H.

In some embodiments of Formula (1) wherein R² and R³ are taken together to form a methylenedioxy group, and R¹ and R⁴ are both H, R′ is C₁-C₆ alkyl. In some embodiments wherein R² and R³ are taken together to form a methylenedioxy group, and R¹ and R⁴ are both H, R′ is not H. In some embodiments wherein R² and R³ are taken together to form a methylenedioxy group and R′ is H, either one or both of R¹ and R⁴ is other than H (i.e., is not H).

In some embodiments, the compound of Formula (1) is not any of:

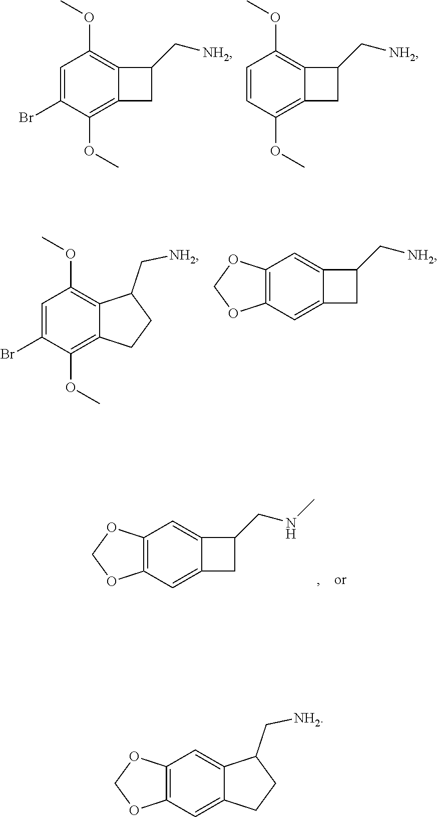

In some embodiments, the compound of Formula (1) is not

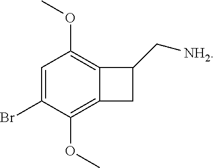

In some embodiments, the compound of Formula (1) is not

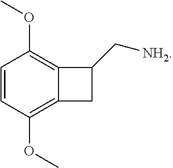

In some embodiments, the compound of Formula (1) is not

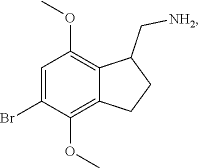 [SMILES: COc1cc(Br)c(OC)c2c1C(CN)CC2]

In some embodiments, the compound of Formula (1) is not

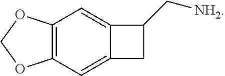 [SMILES: NCC1Cc2cc3c(cc21)OCO3]

In some embodiments, the compound of Formula (1) is not

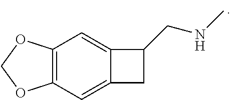

In some embodiments, the compound of Formula (1) is not

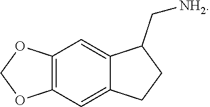 [SMILES: NCC1CCc2cc3c(cc21)OCO3]

In some embodiments, the compound of Formula (1) has the structure of Formula (1A):

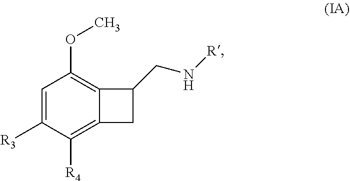

or a pharmaceutically acceptable salt, stereoisomer, hydrate, solvate, or isotopic derivative thereof.

In some embodiments of Formula (1A), R³ is H, Br, Cl, I, C₁-C₆ alkyl, C₁-C₆ alkylthio, or C₃-C₆ cycloalkyl. In some embodiments, R³ is H. In other embodiments, R³ is halogen (e.g., R³ is Br, Cl, or I). In some embodiments, R³ is Cl, I, C₁-C₆ alkyl, C₁-C₆ alkylthio, or C₃-C₆ cycloalkylmethyl. In some embodiments, R³ is Br. In some embodiments, R³ is other than Br (i.e., R³ is not Br). In some embodiments, R³ is Cl or I. In some embodiments, R³ is Cl. In some embodiments, R³ is I. In some embodiments, R³ is C₁-C₆ alkyl. In some such embodiments, R³ is butyl (e.g., R³ is n-butyl, isobutyl, sec-butyl, or tert-butyl). In some embodiments, R³ is isobutyl. In some embodiments, R³ is C₁-C₆ alkylthio. In embodiments, R³ is C₃-C₆ cycloalkyl (e.g., R³ is cyclopropyl, cyclobutyl, cyclopentyl, or cyclohexyl). In embodiments, R³ is cyclopropylmethyl. In other embodiments, R³ is taken together with R² to form a methylenedioxy group.

In some embodiments of Formula (1A), R′ is H.

In some embodiments of Formula (1A), R⁴ is H. In some embodiments, R⁴ is H or —OCH₃. In some embodiments, R⁴ is H or —CH₂CH₂OCH₃. In some embodiments, R⁴ is not H. In some embodiments, R⁴ is —OCH₃ or —CH₂CH₂OCH₃. In some embodiments, R⁴ is —OCH₃. In other embodiments, R⁴ is —CH₂CH₂OCH₃.

In some embodiments of Formula (1A), R³ is Cl, I, C₁-C₆ alkyl, C₁-C₆ alkylthio, or C₃-C₆ cycloalkylmethyl; R⁴ is —OCH₃; and R′ is H. In some such embodiments, R³ is Cl or I; R⁴ is —OCH₃; and R′ is H. In other embodiments, R³ is C₁-C₆ alkyl (e.g., isobutyl); R⁴ is —OCH₃; and R′ is H. In yet other embodiments, R³ is C₃-C₆ cycloalkylmethyl (e.g., cyclopropylmethyl); R⁴ is —OCH₃; and R′ is H.

In some embodiments of Formula (1A) wherein R³ is Br, R⁴ is other than —OCH₃. In some embodiments wherein R³ is Br, R⁴ is not —OCH₃. In some embodiments wherein R³ is Br, R⁴ is H or —CH₂CH₂OCH₃. In some embodiments wherein R³ is Br, R⁴ is H. In some embodiments wherein R³ is Br, R⁴ is —CH₂CH₂OCH₃. In some embodiments wherein R³ is Br, R¹ and R⁴ are not both-OCH₃. In some embodiments wherein R³ is Br, R′ is not H. In some embodiments wherein R³ is Br, R′ is C₁-C₆ alkyl. In some embodiments, R³ is not Br.

In some embodiments of Formula (1A) wherein R³ is H, R⁴ is other than —OCH₃. In some embodiments wherein R³ is H, R⁴ is not —OCH₃. In some embodiments wherein R³ is H, R⁴ is H or —CH₂CH₂OCH₃. In some embodiments wherein R³ is H, R⁴ is H. In some embodiments wherein R³ is H, R⁴ is —CH₂CH₂OCH₃. In some embodiments wherein R³ is H, R¹ and R⁴ are not both —OCH₃. In some embodiments wherein R³ is H, R′ is not H. In some embodiments wherein R³ is H, R′ is C₁-C₆ alkyl. In some embodiments wherein R³ is H, n is 2. In embodiments, R³ is not H.

In some embodiments of Formula (1A) wherein R³ is H or Br, R⁴ is other than —OCH₃. In some embodiments wherein R³ is H or Br, R⁴ is not —OCH₃. In some embodiments wherein R³ is H or Br, R⁴ is H or —CH₂CH₂OCH₃. In some embodiments wherein R³ is H or Br, R⁴ is H. In some embodiments wherein R³ is H or Br, R⁴ is —CH₂CH₂OCH₃. In some embodiments wherein R³ is H or Br, R¹ and R⁴ are not both-OCH₃. In some embodiments wherein R³ is H or Br, R′ is not H. In some embodiments wherein R³ is H or Br, R′ is C₁-C₆ alkyl. In some embodiments wherein R³ is H or Br, n is 2. In embodiments, R³ is not H.

In some embodiments, the compound of Formula (1A) is not any of:

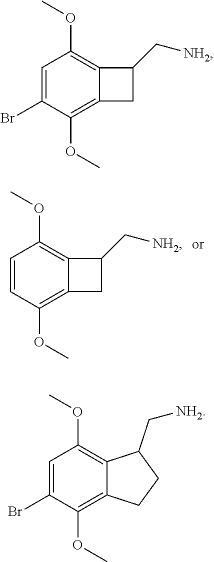

In some embodiments, the compound of Formula (1A) is not

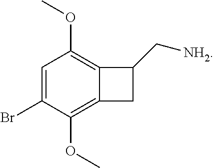

In some embodiments, the compound of Formula (1A) is not

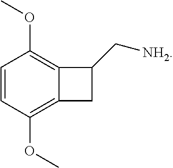

In some embodiments, the compound of Formula (1A) is not

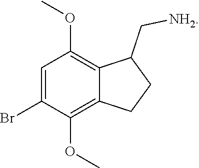 [SMILES: COc1cc(Br)c(OC)c2c1C(CN)CC2]

In some embodiments, the compound has the structure of Formula (1A-I):

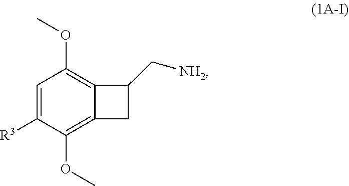

or a pharmaceutically acceptable salt, stereoisomer, hydrate, solvate, or isotopic derivative thereof.

In some embodiments of Formula (1A-I), R³ is H, Br, Cl, I, C₁-C₆ alkyl, C₁-C₆ alkylthio, or C₃-C₆ cycloalkyl. In some embodiments, R³ is H. In other embodiments, R³ is halogen (e.g., R³ is Br, Cl, or I). In some embodiments, R³ is Cl, I, C₁-C₆ alkyl, C₁-C₆ alkylthio, or C₃-C₆ cycloalkylmethyl. In some embodiments, R³ is Br. In some embodiments, R³ is other than Br (i.e., R³ is not Br). In some embodiments, R³ is Cl or I. In some embodiments, R³ is Cl. In some embodiments, R³ is I. In some embodiments, R³ is C₁-C₆ alkyl. In some such embodiments, R³ is butyl (e.g., R³ is n-butyl, isobutyl, sec-butyl, or tert-butyl). In some embodiments, R³ is isobutyl. In some embodiments, R³ is C₁-C₆ alkylthio. In some embodiments, R³ is C₃-C₆ cycloalkyl (e.g., R³ is cyclopropyl, cyclobutyl, cyclopentyl, or cyclohexyl). In some embodiments, R³ is cyclopropylmethyl.

In some embodiments, the compound of Formula (1) has the structure of Formula (1B):

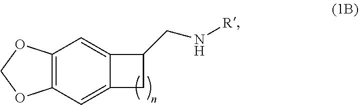

or a pharmaceutically acceptable salt, stereoisomer, hydrate, solvate, or isotopic derivative thereof.

In some embodiments of Formula (1B), R′ is H or methyl. In some embodiments, R′ is H. In other embodiments, R′ is methyl.

In some embodiments of Formula (1B), n is 1 or 2. In some embodiments, n is 1. In other embodiments, n is 2.

In some embodiments of Formula (1B), R′ is H and n is 1. In other embodiments, R′ is H and n is 2. In other embodiments, R′ is methyl and n is 1. In yet other embodiments, R′ is methyl and n is 2.

In some embodiments, the compound of Formula (1B) is not any of:

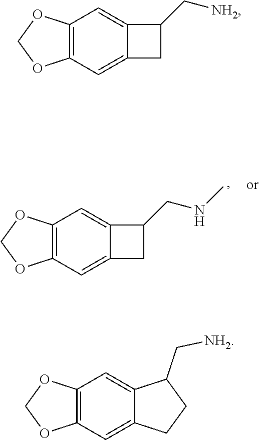

In some embodiments, the compound of Formula (1B) is not

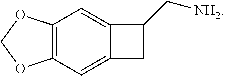 [SMILES: NCC1Cc2cc3c(cc21)OCO3]

In some embodiments, the compound of Formula (1B) is not

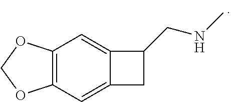

In some embodiments, the compound of Formula (1B) is not

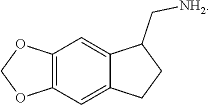 [SMILES: NCC1CCc2cc3c(cc21)OCO3]

In some embodiments, the compound is selected from Table 1, or a pharmaceutically acceptable salt, stereoisomer, hydrate, solvate, or isotopic derivative thereof.

TABLE 1
Exemplary Compounds of Formula (1)

| No. | Structure |
|---|---|
| 1 | 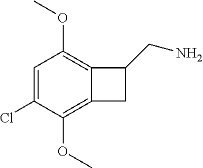 |
| 2 | 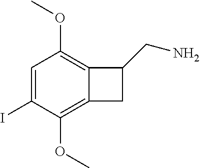 |
| 3 | 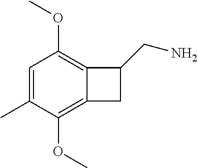 |
| 4 | 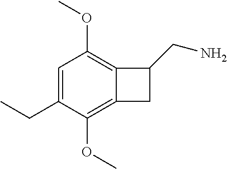 [SMILES: CCc1cc(OC)c2c(c1OC)CC2CN] |
| 5 | 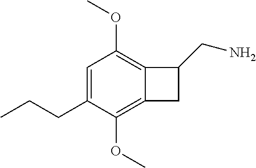 [SMILES: CCCc1cc(OC)c2c(c1OC)CC2CN] |
| 6 | 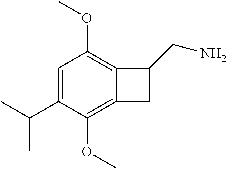 [SMILES: COc1cc(C(C)C)c(OC)c2c1C(CN)C2] |
| 7 | 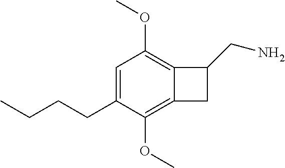 [SMILES: CCCCc1cc(OC)c2c(c1OC)CC2CN] |
| 8 | 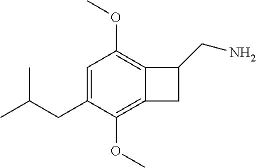 [SMILES: COc1cc(CC(C)C)c(OC)c2c1C(CN)C2] |
| 9 | 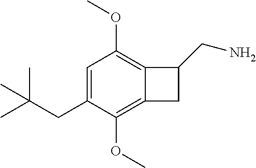 [SMILES: COc1cc(CC(C)(C)C)c(OC)c2c1C(CN)C2] |
| 10 | 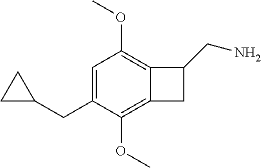 [SMILES: COc1cc(CC2CC2)c(OC)c2c1C(CN)C2] |
| 11 | 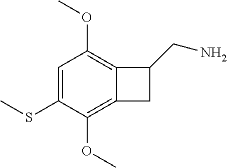 [SMILES: COc1cc(SC)c(OC)c2c1C(CN)C2] |
| 12 | 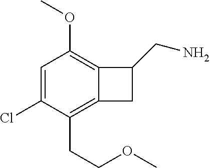 [SMILES: COCCc1c(Cl)cc(OC)c2c1CC2CN] |
| 13 | 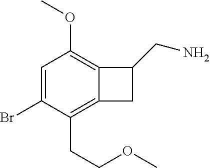 [SMILES: COCCc1c(Br)cc(OC)c2c1CC2CN] |
| 14 | 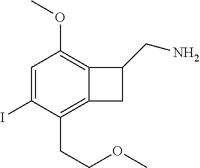 [SMILES: COCCc1c(I)cc(OC)c2c1CC2CN] |
| 15 | 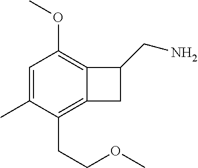 [SMILES: COCCc1c(C)cc(OC)c2c1CC2CN] |
| 16 | 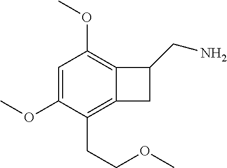 [SMILES: COCCc1c(OC)cc(OC)c2c1CC2CN] |
| 17 | 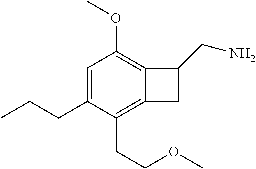 [SMILES: CCCc1cc(OC)c2c(c1CCOC)CC2CN] |
| 18 | 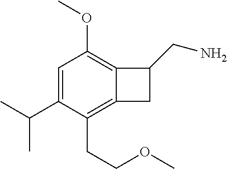 [SMILES: COCCc1c(C(C)C)cc(OC)c2c1CC2CN] |
| 19 | 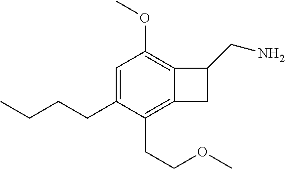 [SMILES: CCCCc1cc(OC)c2c(c1CCOC)CC2CN] |
| 20 | 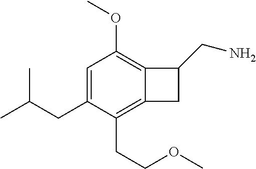 [SMILES: COCCc1c(CC(C)C)cc(OC)c2c1CC2CN] |
| 21 | 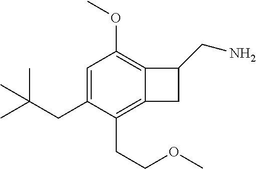 [SMILES: COCCc1c(CC(C)(C)C)cc(OC)c2c1CC2CN] |
| 22 | 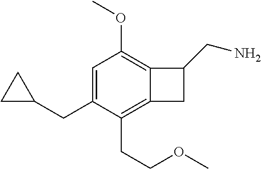 [SMILES: COCCc1c(CC2CC2)cc(OC)c2c1CC2CN] |
| 23 | 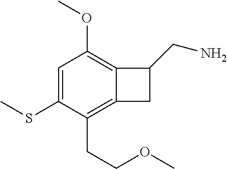 [SMILES: COCCc1c(SC)cc(OC)c2c1CC2CN] |
| 24 | |

In some embodiments, the compound is selected from Table 2, or a pharmaceutically acceptable salt, stereoisomer, hydrate, solvate, or isotopic derivative thereof.

TABLE 2
Exemplary Compounds of Formula (1)

| No. | Structure |
|---|---|
| 26 | 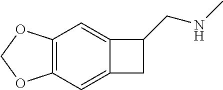 |
| 27 | 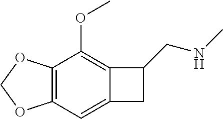 [SMILES: CNCC1Cc2cc3c(c(OC)c21)OCO3] |
| 28 | 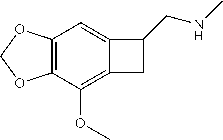 [SMILES: CNCC1Cc2c1cc1c(c2OC)OCO1] |
| 29 | 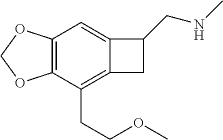 [SMILES: CNCC1Cc2c1cc1c(c2CCOC)OCO1] |
| 30 | 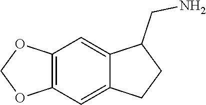 [SMILES: NCC1CCc2cc3c(cc21)OCO3] |
| 31 | 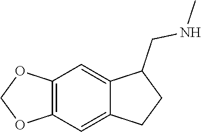 |
| 32 | 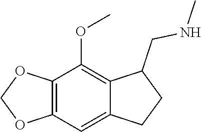 [SMILES: CNCC1CCc2cc3c(c(OC)c21)OCO3] |
| 33 | 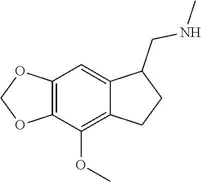 [SMILES: CNCC1CCc2c1cc1c(c2OC)OCO1] |
| 34 | 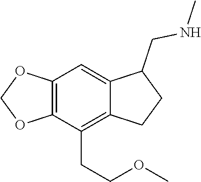 [SMILES: CNCC1CCc2c1cc1c(c2CCOC)OCO1] |

In some embodiments, the compound is:

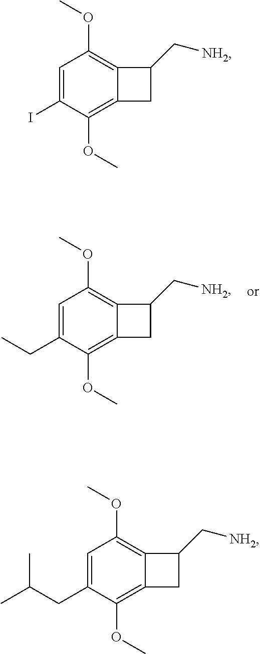 [SMILES: COc1cc(I)c(OC)c2c1C(CN)C2 ; CCc1cc(OC)c2c(c1OC)CC2CN ; COc1cc(CC(C)C)c(OC)c2c1C(CN)C2]

or a pharmaceutically acceptable salt, stereoisomer, hydrate, solvate, or isotopic derivative thereof.

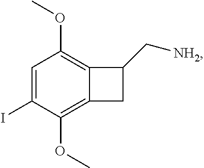

In some embodiments, the compound is or a pharmaceutically acceptable salt, stereoisomer, hydrate, solvate, or isotopic derivative thereof.

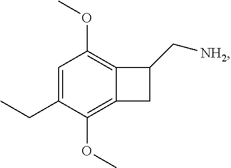 [SMILES: CCc1cc(OC)c2c(c1OC)CC2CN]

In some embodiments, the compound is or a pharmaceutically acceptable salt, stereoisomer, hydrate, solvate, or isotopic derivative thereof.

In some embodiments, the compound is

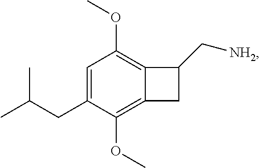 [SMILES: COc1cc(CC(C)C)c(OC)c2c1C(CN)C2]

or a pharmaceutically acceptable salt, stereoisomer, hydrate, solvate, or isotopic derivative thereof.

The individual compounds of the disclosed compositions will be understood to also encompass pharmaceutically acceptable salts of such compounds. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. One of ordinary skill in the art can select from among a wide variety of available counterions those that are pharmaceutically acceptable. In specific applications, the selection of a given anion or cation for preparation of a salt may result in increased or decreased solubility of that salt. Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge, et al., J. Pharm. Sci. 1997, 66, 1-19.)

Certain compounds disclosed herein may contain one or more ionizable groups (groups from which a proton can be removed (e.g., —COOH) or added (e.g., amines) or which can be quaternized (e.g., amines)). All possible ionic forms of such molecules and salts thereof are included in the present disclosure.

A disclosed compound can exist in solid or liquid form. In the solid state, the compound may exist in crystalline or noncrystalline form, or as a mixture thereof. The skilled artisan will appreciate that pharmaceutically acceptable solvates may be formed for crystalline or non-crystalline compounds. In crystalline solvates, solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The subject matter described herein includes such solvates.

The skilled artisan will further appreciate that certain compounds described herein that exist in crystalline form, including the various solvates thereof, may exhibit polymorphism (i.e. the capacity to occur in different crystalline structures). These different crystalline forms are typically known as “polymorphs.” The subject matter disclosed herein includes such polymorphs. Polymorphs include the different crystal packing arrangements of the same elemental composition of a compound. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. Various factors such as the recrystallization solvent, rate of crystallization, and storage temperature may cause a single crystal form to dominate. In addition, one polymorph may spontaneously convert to another polymorph under certain conditions.

The compounds described herein may contain one or more asymmetric centers and give rise to enantiomers, diastereomers, and other stereoisomeric forms. Each chiral center may be defined, in terms of absolute stereochemistry, as (R)- or (S)-. The invention includes all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. Various methods are known in the art for preparing optically active forms and determining activity. Such methods include standard tests described herein and other similar tests which are well known in the art. Examples of methods that can be used to obtain optical isomers of the compounds according to the present disclosure include selective crystallization, enzymatic resolution, asymmetric synthesis (including asymmetric chemical synthesis and asymmetric enzymatic synthesis), kinetic resolution, and chiral chromatography cl (including chiral liquid chromatography, gas chromatography, and high-performance liquid chromatography). When the compounds described herein contain olefinic double bonds or other centers of geometric asymmetry, and unless specified otherwise, it is intended that the compounds include both E and Z geometric isomers. Likewise, tautomeric forms are included.

Compounds of the present disclosure possess a chiral center on the benzocycloalkene ring, as shown by the asterisk (*) in Formula (1) below.

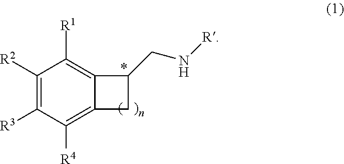

In some embodiments, a compound of the disclosure, or a pharmaceutically acceptable salt, stereoisomer, or isotopic derivative has the structure of Formula (1), Formula (1A), or Formula (1B), wherein the stereochemistry at the * carbon is S. In preferred embodiments, the stereochemistry at the * carbon is R. For example, in some preferred embodiments, wherein the compound has the structure of Formula (1), the stereochemistry at the * carbon is R, as shown below in Formula (R-1):

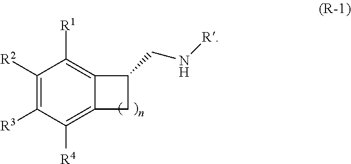

In certain preferred embodiments, wherein the compound has the structure of Formula (1A), the stereochemistry at the * carbon is R, as shown below in Formula (R-1A):

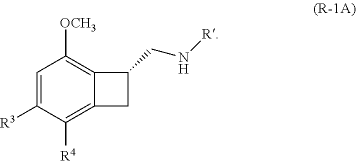

In some embodiments, exemplary compounds of Formula (R-1A) include:

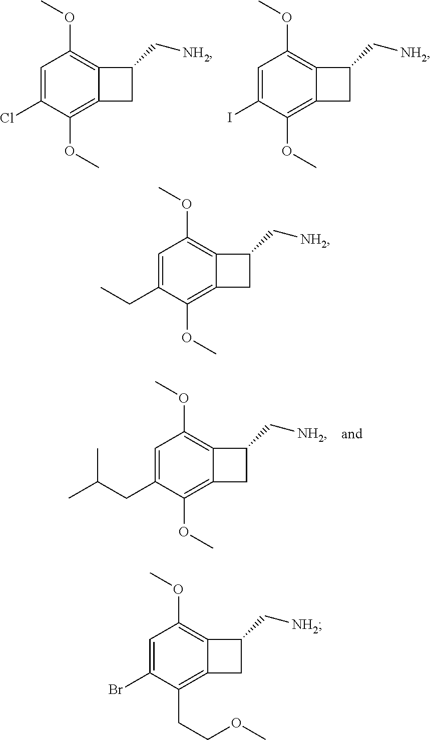 [SMILES: COc1cc(Cl)c(OC)c2c1[C@H](CN)C2 ; COc1cc(I)c(OC)c2c1[C@H](CN)C2 ; CCc1cc(OC)c2c(c1OC)C[C@H]2CN ; COc1cc(CC(C)C)c(OC)c2c1[C@H](CN)C2 ; COCCc1c(Br)cc(OC)c2c1C[C@H]2CN]

as well as pharmaceutically acceptable salts, hydrates, solvates, and isotopic derivatives thereof.

In other embodiments, wherein the compound has the structure of Formula (1A), the stereochemistry at the * carbon is S.

In some embodiments, wherein the compound has the structure of Formula (1B), the stereochemistry at the * carbon is R, as shown below in Formula (R-1B):

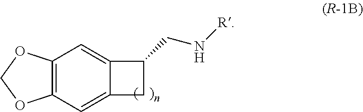

In some embodiments, exemplary compounds of Formula (R-1B) include:

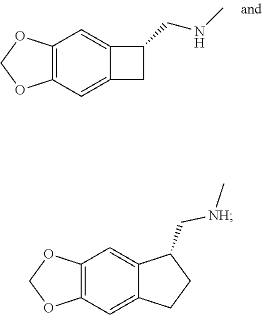

as well as pharmaceutically acceptable salts, hydrates, solvates, and isotopic derivatives thereof.

In other preferred embodiments, wherein the compound has the structure of Formula (1B), the stereochemistry at the * carbon is S, as shown below in Formula (S-1B):

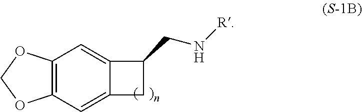

In some embodiments, exemplary compounds of Formula (S-1B) include:

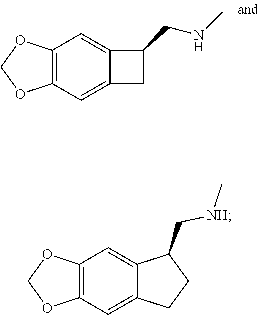

as well as pharmaceutically acceptable salts, hydrates, solvates, and isotopic derivatives thereof.

When the compounds described herein contain olefinic double bonds or other centers of geometric asymmetry, and unless specified otherwise, it is intended that the compounds include both E and Z geometric isomers. Likewise, tautomeric forms are included.

In some embodiments, a disclosed compound is provided as a racemic mixture (e.g., a substance comprising an equimolar amount of both R and S isomers). In some embodiments, a disclosed compound is provided as a non-racemic mixture (e.g., a substance comprising an enantiomeric excess of either the R or S isomer). For example, wherein the non-racemic mixture comprises an enantiomeric excess of the R isomer, in some embodiments the enantiomeric excess of the Risomer is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the enantiomeric excess of the R isomer is about 10%-99%, 20%-99%, 30%-99%, 40%-99%, 50%-99%, 55%-99%, 60%-99%, 65%-99%, 70%-99%, or 75%-99%, wherein each range is inclusive. In some embodiments, the enantiomeric excess of the R isomer is about 10%, 15%, 20%, 25%, 30%, 35%, 40%, 45%, 50%, 55%, 60%, 65%, 70%, 75%, 80%, 85%, 90%, 95%, or 99%. In some embodiments, wherein the non-racemic mixture comprises an enantiomeric excess of the S isomer, in some embodiments the enantiomeric excess of the S isomer is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the enantiomeric excess of the S isomer is about 10%-99%, 20%-99%, 30%-99%, 40%-99%, 50%-99%, 55%-99%, 60%-99%, 65%-99%, 70%-99%, or 75%-99%, wherein each range is inclusive. In some embodiments, the enantiomeric excess of the S isomer is about 10%, 15%, 20%, 25%, 30%, 35%, 40%, 45%, 50%, 55%, 60%, 65%, 70%, 75%, 80%, 85%, 90%, 95%, or 99%. In some embodiments, the non-racemic mixture comprises a single stereoisomer. In some embodiments, the non-racemic mixture comprises the R-isomer as an enantiopure compound. In some embodiments, the non-racemic mixture comprises the S-isomer as an enantiopure compound.

The invention also includes compounds with at least one desired isotopic substitution of an atom, at an amount above the natural abundance of the isotope, i.e., isotopically enriched. Isotopes are atoms having the same atomic number but different mass numbers, i.e., the same number of protons but a different number of neutrons. Examples of isotopes that can be incorporated into compounds described herein include isotopes of hydrogen, carbon, nitrogen, oxygen, and chlorine such as ²H, ³H, ¹¹C, ¹³C, ¹⁴C, ¹⁵N, ¹⁷O, ¹⁸O, and ³⁶Cl respectively. In one non-limiting embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F-labeled compound may be particularly desirable for PET or SPECT studies. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of this invention can generally be prepared by carrying out the procedures disclosed in the schemes or in the examples and preparations described below by substituting a readily available isotopically labeled reagent for a non-isotopically labeled reagent.

The invention also includes prodrugs of disclosed compounds. A “prodrug” is a precursor of a biologically active pharmaceutical agent, which may undergo a chemical or a metabolic conversion to become the biologically active agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent. Typical examples of prodrugs include compounds with biologically labile or cleavable (protecting) groups on a functional moiety of the active compound. Prodrugs include compounds that can be oxidized, reduced, aminated, deaminated, hydroxylated, dehydroxylated, hydrolyzed, alkylated, dealkylated, acylated, deacylated, phosphorylated, or dephosphorylated to produce the active compound. Commonly used functional groups include esters, carbonates, carbamates, amides, phosphates, and sulfonamides. These functional groups can be attached to the drug molecule via a linker that is designed to be cleaved under specific physiological conditions, such as enzymatic hydrolysis or pH-dependent cleavage. The choice of functional group depends on factors such as stability, ease of synthesis, enzymatic activity, and desired rate of prodrug conversion.

Generally, the individual disclosed compounds will be administered as part of a pharmaceutical composition or formulation, and are prepared for inclusion in such composition or formulations as isolated or purified compounds. The terms “isolated,” “purified,” or “substantially pure,” as used herein, refer to material that is substantially or essentially free from components that normally accompany the material when the material is synthesized, manufactured, or otherwise produced. An “isolated,” “purified,” or “substantially pure” preparation of a compound is accordingly defined as a preparation having a chromatographic purity (of the desired compound) of greater than 90%, more preferably greater than 95%, more preferably greater than 96%, more preferably greater than 97%, more preferably greater than 98%, more preferably greater than 99%, more preferably greater than 99.5%, and most preferably greater than 99.9%, as determined by area normalization of an HPLC profile or other similar detection method.

Preferably the substantially pure compound used in the invention is substantially free of any other active compounds which are not intended to be administered to a subject. In this context “substantially free” can be taken to mean that no active compound(s) other than the active compound intended to be administered to a subject are detectable by HPLC or other similar detection method, or are below a desired threshold of detection such as defined above.

In some aspects, features of disclosed compounds provide various advantages. Such advantages may be related to modulation of neurotransmission, pharmacokinetics, such as properties related to absorption, distribution, metabolism, and excretion of a disclosed compound, and subjective effects, such as upon administration to a subject. In some embodiments, such advantages are determined relative to a comparator.

In some embodiments, the comparator is an analogous phenethylamine that is not conformationally restricted. For example, in some embodiments, the comparator is a 2C-X compound. In some embodiments, the comparator is 2C-B. In some embodiments, the comparator is 2C-C. In some embodiments, the comparator is 2C-I. In some embodiments, the comparator is chosen on the basis of shared structural features with a disclosed compound.

For example, in some embodiments wherein a disclosed compound has the structure of

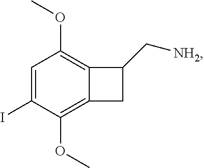

a suitable comparator may be

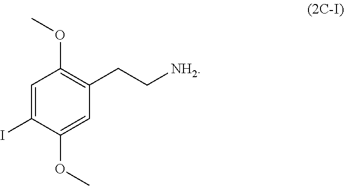

In some embodiments, wherein a disclosed compound has the structure of

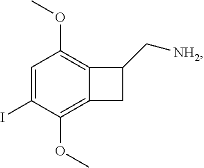

a suitable comparator may be

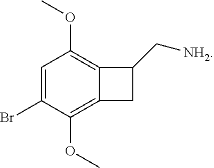

In another example, in some embodiments wherein a disclosed compound has the structure of

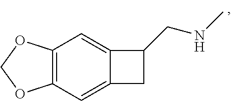

a suitable comparator may be

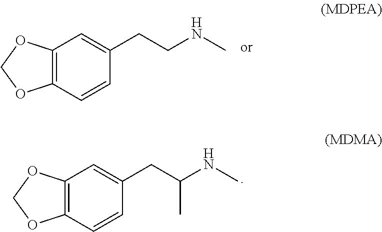

### C. Methods of Preparing Disclosed Compounds

In some aspects, provided herein are methods of preparing disclosed compounds, such as compounds of Formula (1), Formula (1A), and Formula (1B). Exemplary synthetic methods are provided below, and in Examples 12-14.

In some embodiments, a disclosed compound can be prepared from a 2-nitrobenzoic acid precursor as shown below.

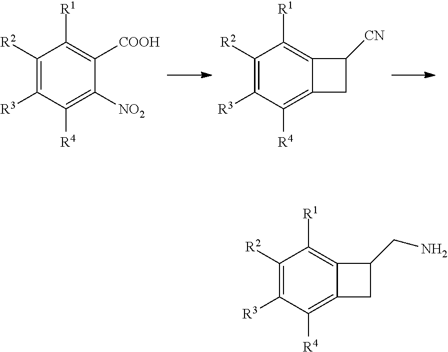

In a first reaction sequence, a 2-nitrobenzoic acid precursor is converted to a benzocyclobutenecarbonitrile intermediate. This transformation is accomplished by i) reduction of the nitro group (e.g., by catalytic hydrogenation); ii) diazotization of the resulting anthranilic acid to produce a benzenediazonium-2-carboxylate intermediate; iii) thermal decomposition of the benzenediazonium-2-carboxylate intermediate to the corresponding benzyne; and iv) 2+2 cycloaddition with acrylonitrile. The benzocyclobutenecarbonitrile is then reduced to the corresponding 1-aminomethyl compound (e.g., by hydrogenation over Raney nickel).

In some embodiments, a disclosed compound is prepared from a 1-chlorobenzocyclobutene precursor as shown below.

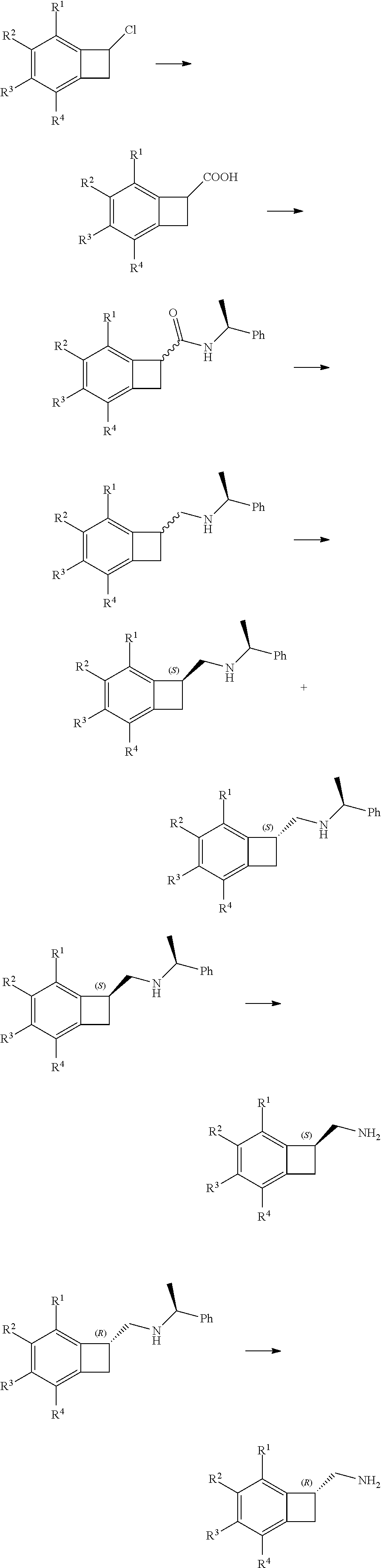

A 1-chlorobenzocyclobutene precursor is converted to the corresponding Grignard reagent (e.g., by addition of Mg in THF), then quenched with CO₂ to provide a benzocyclobutene-1-carboxylic acid intermediate. Condensation of this intermediate with (S)-1-phenethylamine gives a mixture of diastereomeric amides. The amides are then reduced to yield a diastereomeric mixture of (S,S) and (S,R) N−1-phenethylamines. These diastereomeric amines are then separated (e.g., by radial chromatography or HPLC). Subsequent catalytic debenzylation provides enantiopure compounds of the present disclosure.

In some embodiments, for certain compounds of the disclosure (e.g., wherein R³ is Br), R³ may not be present in a 2-nitrobenzoic acid precursor or a 1-chlorobenzocyclobutene precursor. Rather, R³ can be introduced by direct substitution of the phenyl ring later in the synthesis. For example, certain compounds of the disclosure wherein R² is H and R³ is Br are synthesized according to the following general reaction procedures.

In some embodiments, exemplary compounds wherein R² is H and R³ is Br are synthesized from a 2-nitrobenzoic acid precursor as shown below:

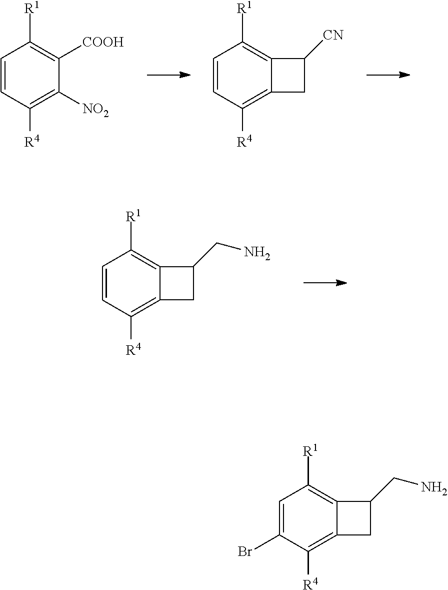

In some embodiments, exemplary compounds wherein R² is H and R³ is Br are synthesized from a 1-chlorobenzocyclobutene precursor as shown below:

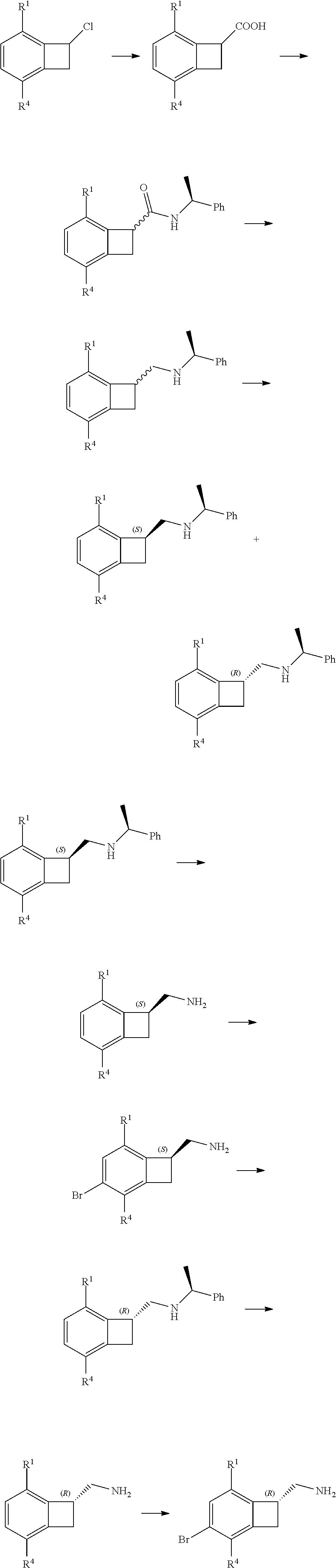

The reaction sequences are performed as described above, with the addition of a final bromination step that directly brominates the phenyl ring to install Br as R³ (e.g., using N-bromosuccinimide, or Br₂ in glacial acetic acid). Similar synthetic routes may be utilized to access other disclosed compounds (e.g., wherein R³ is Cl or I), in racemic or enantiopure form.

Additional procedures known in the art that are useful for preparing the disclosed compounds may be found in McLean, et al., J. Med. Chem. 2006, 49, 4269-4274 and 5794-5803.

Additional methods for synthesis of the compounds described herein and any necessary starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the disclosed compounds. In general, the approaches used for similar compounds may be used (e.g., TiHKAL; Glennon et al., J. Med. Chem., 1986; 29(2), 194-199; Nichols et al. 1991. J. Med. Chem., 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European J. Med. Chem., 138, 1002-1033; Pérez-Silanes et al. 2001. J. Heterocyclic Chem, 38(5), 1025-1030; and references therein), such adaptation being that known and understood to those of ordinary skill; see also Brandt et al. (2011). Drug Test Anal., 4:24-32.

### D. Pharmaceutical Compositions

In some aspects, provided herein are compositions, such as pharmaceutical compositions, comprising the disclosed compounds, such as compounds of Formula (I). “Pharmaceutical compositions” are compositions that include the disclosed compound(s) together in an amount (for example, in a unit dosage form) with a pharmaceutically acceptable carrier, diluent, or excipient. Some embodiments will not have a single carrier, diluent, or excipient alone, but will include multiple carriers, diluents, and/or excipients. Compositions can be prepared by standard pharmaceutical formulation techniques such as disclosed in, e.g., Remington: The Science & Practice of Pharmacy (2020) 23th ed., Academic Press., Cambridge, Mass.; The Merck Index (1996) 12th ed., Merck Pub. Group, Whitehouse, N.J.; Pharm. Principles of Solid Dosage Forms (1993), Technomic Pub. Co., Inc., Lancaster, Pa.; and Ansel & Stoklosa, Pharm. Calculations (2001) 11th ed., Lippincott Williams & Wilkins, Baltimore, Md.; & Poznansky et al. Drug Delivery Systems (1980), R. L. Juliano, ed., Oxford, N.Y., pp. 253-315).

“Pharmaceutically acceptable” used in connection with an excipient, carrier, diluent, or other ingredient means the ingredient is generally safe and, within the scope of sound medical judgment, suitable for use in contact with cells of humans and animals without undue toxicity, irritation, allergic response, or complication, commensurate with a reasonable risk/benefit ratio.

In some embodiments, pharmaceutical compositions comprising a disclosed compound can be administered by a variety of routes including oral, mucosal (e.g., buccal, sublingual), rectal, transdermal, subcutaneous, intravenous, intramuscular, inhaled, and intranasal. In some embodiments, the compounds employed in the methods of this invention are effective as oral, mucosal (e.g., buccal, sublingual), rectal, transdermal, subcutaneous, intravenous, intramuscular, inhaled, and intranasal compositions. Such compositions are prepared in a manner well known in the pharmaceutical art and comprise at least one active compound. (See, e.g., Remington, 2020.)

The disclosed compositions are preferably formulated in a unit dosage form, each dosage containing a therapeutically effective amount of the active ingredients, for example in the dosage amounts disclosed below. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose for a “booster” dose as described below), of the pharmaceutical composition administered.

Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis (including the mucosa) of a subject for an extended or brief period of time.

In embodiments, a disclosed composition is formulated in a pharmaceutically acceptable oral dosage form. Oral dosage forms include oral liquid dosage forms (such as tinctures, drops, emulsions, syrups, elixirs, suspensions, and solutions, and the like) and oral solid dosage forms. A disclosed pharmaceutical composition may be prepared as a formulation suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

In some embodiments, a disclosed composition is formulated as an oral solid dosage form. Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations. Accordingly, in some embodiments, the disclosed oral solid dosage forms may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations may be administered as a single capsule or in multiple capsule dosage form. In some embodiments, the pharmaceutical formulation is administered in two, three, four, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

In some embodiments, a disclosed composition is formulated as an oral liquid dosage form. Oral liquid dosage forms include tinctures, drops, emulsions, syrups, elixirs, suspensions, and solutions, and the like. These oral liquid dosage forms may be formulated with any pharmaceutically acceptable excipient known to those of skill in the art for the preparation of liquid dosage forms, and with solvents, diluents, carriers, excipients, and the like chosen as appropriate to the solubility and other properties of the active agents and other ingredients. Solvents may be, for example, water, glycerin, simple syrup, alcohol, medium chain triglycerides (MCT), and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Liquid formulations also may be prepared as single dose or multi-dose beverages. Suspensions may include oils. Such oils include peanut oil, sesame oil, cottonseed oil, corn oil, and olive oil. Suitable oils also include carrier oils such as MCT and long chain triglyceride (LCT) oils. Suspension preparation may also contain esters of fatty acids such as ethyl oleate, isopropyl myristate, fatty acid glycerides, and acetylated fatty acid glycerides. Suspension formulations may include alcohols, (such as ethanol, isopropyl alcohol, hexadecyl alcohol), glycerol, and propylene glycol. Ethers, such as poly(ethylene glycol), petroleum hydrocarbons such as mineral oil and petrolatum, and water may also be used in suspension formulations. Suspension can thus include an aqueous liquid or a non-aqueous liquid, an oil-in-water liquid emulsion, or a water-in-oil emulsion.

In some embodiments, formulations are provided comprising the disclosed compositions and at least one dispersing agent or suspending agent for oral administration to a subject. The formulation may be a powder and/or granules for suspension, and upon admixture with water, a substantially uniform suspension is obtained. The aqueous dispersion can comprise amorphous and non-amorphous particles consisting of multiple effective particle sizes such that a drug is absorbed in a controlled manner over time.

Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the disclosed compounds, the liquid dosage forms may comprise additives, such as one or more (a) disintegrating agents, (b) dispersing agents, (c) wetting agents, (d) preservatives, (e) viscosity enhancing agents, (f) sweetening agents, or (g) flavoring agents.

Disclosed compositions also may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

In embodiments, disclosed pharmaceutical compositions may be formulated into a topical dosage form. Topical dosage forms include transmucosal and transdermal formulations, such as aerosols, emulsions, sprays, ointments, salves, gels, pastes, lotions, liniments, oils, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which may be used include Vaseline®, lanolin, PEG, alcohols, transdermal enhancers, and combinations thereof.

In some embodiments, a disclosed pharmaceutical composition may be formulated in an ophthalmic formulation. Ophthalmic formulations of the disclosure include topical formulations, such as eye drops, gels, and ointments. Ophthalmic formulations may contain one or more viscosity-modifying agents and have a viscosity that feels comfortable to the eye and do not cause blurring of the vision. For example, an ophthalmic formulation may have a viscosity of 1.0 to 100,000 cP (e.g., from 2.0 to 90,000 cP or from 2.5 and 75,000 cP). Viscosity-modifying agents are substances that have the ability to cause thickening (increase the viscosity) of ophthalmic formulations. Viscosity modifying agents include xanthan gum, edetate, methylcellulose, carboxymethylcellulose, hydroxypropyl methylcellulose, hydroxyethyl cellulose, polyethylene glycol, propylene glycol alginate, chitosan, and tragacanth. Hydrogels may also be used as viscosity-enhancing excipients, particularly in artificial tears. Compatible viscosity-adjusting agents can be used in all formulations mentioned herein. Concentrations of viscosity-modifying agents in ophthalmic formulations of the disclosure can range from about 0.1 percent to about 10 percent by weight (e.g., between 1 percent and 5 percent by weight). Sorbitol may be used as a combined tonicity-adjusting and viscosity-modifying excipient. Sorbitol may be used in ophthalmic formulations of the disclosure in a concentration range from about 0.1 to about 10 percent (e.g., from 2 percent to 5 percent by weight).

Additional ophthalmic formulations of the disclosure include contact lenses. In some embodiments, a disclosed compound or pharmaceutical composition is incorporated into a contact lens for ocular drug delivery. The contact lens may be a hydrogel contact lens or a molecularly imprinted contact lens. Another exemplary contact lens drug delivery system known to those of skill in the art is the experimental SIGHT (Sustained Innovative Glaucoma and Ocular Hypertension Treatment) treatment, which seeks to treat mild to moderate glaucoma and ocular hypertension (see Clinical Trial NCT04747808). The SIGHT drug-eluting lens for glaucoma treatment incorporates the FDA-approved drug bimatoprost into contact lenses that are formulated for controlled drug release. The SIGHT lens comprises drug and barrier layers on the lens surface to control the diffusion release kinetics of the drug. Ophthalmic formulations of the disclosure include those of similar material design as the SIGHT lens, as well as others generally known to those of skill in the art (e.g., as described in Franco, et al., Polymers, 2021, 13, 1102).

### E. Pharmaceutical Combinations

It should be readily appreciated that the disclosed compositions are not limited to combinations of a single compound, or (when formulated as a pharmaceutical composition) limited to a single carrier, diluent, and/or excipient alone, but may also include combinations of multiple compounds (including additional active compounds), and/or multiple carriers, diluents, and excipients. Pharmaceutical compositions of this invention thus may comprise a compound of Formula (1) together with one or more other active agents (or their derivatives and analogs) in combination, together with one or more pharmaceutically-acceptable carriers, diluents, and/or excipients, and additionally with one or more other active compounds.

In some embodiments, a formulation of the invention will be prepared so as to increase an existing therapeutic effect, provide an additional therapeutic effect, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that may be increased or added in embodiments of the invention include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, empathogenic, psychedelic, sedative, and stimulant effects.

“Synergistic effects” should be understood to include increases in potency, bioactivity, bioaccessibility, bioavailability, or therapeutic effect, that are greater than the additive contributions of the components acting alone. Numerous methods known to those of skill in the art exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang, Front Pharmacol., 2019; 10:1222), or the equation of Loewe additivity (Loewe & Muischnek, 1926, Arch. Exp. Pathol Pharmacol. 114:313-326). A synergistic effect also may be calculated using methods such as the Sigmoid-Emax equation (Holford & Scheiner, 1981, Clin. Pharmacokinet. 6:429-453) and the median-effect equation (Chou & Talalay, 1984, Adv. Enzyme Regul. 22:27-55). The corresponding graphs associated with the equations referred to above are the concentration-effect curve and combination index curve, respectively. Each equation referred to above may be applied to experimental data to generate a corresponding graph to aid in assessing the effects of the drug combination.

In embodiments, a disclosed pharmaceutical composition comprises an additional active compound. In some embodiments, the additional active compound is selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, dissociatives, cannabinoids, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, plasticity-inducing agents (e.g., psychoplastogens), monoamine oxidase inhibitors, tryptamines, terpenes, phenethylamines, sedatives, stimulants, serotonergic agents, and vitamins. In some embodiments, the additional active compound acts to increase a therapeutic effect, provide an additional therapeutic effect, decrease an unwanted effect, increase stability or shelf-life, improve bioavailability, induce synergy, increase plasticity (e.g., neural plasticity), or alter pharmacokinetics or pharmacodynamics. In some embodiments, the additional therapeutic effect is an antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, empathogenic, psychedelic, sedative, or stimulant effect.

In some embodiments, an additional active compound is a tryptamine. As understood by those in the art, tryptamines are compounds having the general structure below, wherein R^{N1}, R^{N2}, R^{α}, R^{β}, R², R⁴, R⁵, R⁶, and R⁷ are as defined herein and as generally understood in the art:

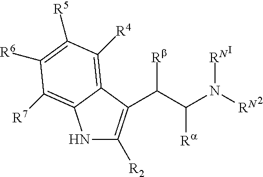

In some embodiments, R^{N1}, R^{N2}, R^{α}, R^{β}, R², R⁴, R⁵, R⁶, and R⁷ are each independently hydrogen, deuterium, halogen (F, Cl, Br, or I), OH, phosphoryloxy, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. Additionally, any two of R^{N1}, R^{N2}, R^{α}, R^{β}, R², R⁴, R⁵, R⁶, and R⁷ and the intervening atoms can be taken together to form an optionally substituted optionally substituted cycloalkyl, optionally substituted cycloalkenyl, optionally substituted aryl, or optionally substituted heterocyclyl. In embodiments, the tryptamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, the additional active compound is a tryptamine selected from the consisting of O-phosphoryl-4-hydroxy-N,N-dimethyltryptamine (psilocybin), group 6-allyl-N,N-diethyl-norlysergamide (AL-LAD), N,N-dibutyltryptamine (DBT), N,N-diethyltryptamine (DET), N,N-diisopropyltryptamine (DiPT), 5-methoxy-α-methyltryptamine (α,O-DMS), N,N-dimethyl-tryptamine (DMT), 2,α-dimethyltryptamine (2,α-DMT), α,N-dimethyltryptamine (α,N-DMT), N,N-dipropyltryptamine (DPT), N-ethyl-N-isopropyltryptamine (EiPT), α-ethyltryptamine (AET), 6,N,N-triethylnorlysergamide (ETH-LAD), 3,4-dihydro-7-methoxy-1-methylcarboline (Harmaline), 7-methoxy-1-methylcarboline (Harmine), N,N-dibutyl-4-hydroxytryptamine (4-HO-DBT), N,N-diethyl-4-hydroxytryptamine (4-HO-DET), N,N-diisopropyl-4-hydroxytryptamine (4-HO-DiPT), 4-hydroxy-N,N,N-trimethyltryptamine (4-HO-TMT), N,N-dimethyl-4-hydroxytryptamine (4-HO-DMT), N,N-dimethyl-5-hydroxytryptamine (5-HO-DMT, bufotenine), N,N-dipropyl-4-hydroxytryptamine (4-HO-DPT), N-ethyl-4-hydroxy-N-methyltryptamine (4-HO-MET), 4-hydroxy-N-isopropyl-N-methyltryptamine (4-HO-MiPT), 4-hydroxy-N-methyl-N-propyl-tryptamine (4-HO-MPT), 4-hydroxy-N,N-tetramethylenetryptamine (4-HO-pyr-T), 12-methoxyibogamine (Ibogaine), N-butyl-N-methyltryptamine (MBT), N,N-diisopropyl-4,5-methylenedioxytryptamine (4,5-MDO-DiPT), N,N-diisopropyl-5,6-methylenedioxytryptamine (5,6-MDO-DiPT), N,N-dimethyl-4,5-methylenedioxytryptamine (4,5-MDO-DMT), N,N-dimethyl-5,6-methylenedioxytryptamine (5,6-MDO-DMT), N-isopropyl-N-methyl-5,6-methylenedioxytryptamine (5,6-MDO-MiPT), N,N-diethyl-2-methyltryptamine (2-Me-DET), 2,N,N-trimethyltryptamine (2-Me-DMT), N-acetyl-5-methoxytryptamine (melatonin), N,N-diethyl-5-methoxytryptamine (5-MeO-DET), N,N-diisopropyl-5-methoxytryptamine (5-MeO-DiPT), N,N,diallyl-5-methoxytryptamine (5-MeO-DALT), 5-methoxy-N,N-dimethyltryptamine (5-MeO-DMT), N-isopropyl-4-methoxy-N-methyltryptamine (4-MeO-MiPT), N-isopropyl-5-methoxy-N-methyltryptamine (5-MeO-MiPT), 5,6-dimethoxy-N-isopropyl-N-methyltryptamine (5,6-MeO-MiPT), 5-methoxy-N-methyl-tryptamine (5-MeO-NMT), 5-methoxy-N,N-tetramethylenetryptamine (5-MeO-pyr-T), 6-methoxy-1-methyl-1,2,3,4-tetrahydrocarboline (6-MeO-THH), 5-methoxy-2,N,N-trimethyl-tryptamine (5-MeO-TMT), N,N-dimethyl-5-methylthiotryptamine (5-MeS-DMT), N-isopropyl-N-methyltryptamine (MiPT), α-methyltryptamine (α-MT), N-ethyltryptamine (NET), N-methyltryptamine (NMT), 6-propylnorlysergamide (PRO-LAD), N,N-tetra-methylenetryptamine (pyr-T), Tryptamine (T), 7-methoxy-1-methyl-1,2,3,4-tetrahydrocarboline (Tetrahydroharmine), or α,N-dimethyl-5-methoxytryptamine (α,N,O-TMS), or a pharmaceutically acceptable salt, hydrate, solvate, prodrug, stereoisomer, or tautomer thereof, or a combination thereof.

In some embodiments, an additional tryptamine will be a “complex tryptamine” or other indolamine and including such examples as iboga alkaloids such as ibogaine, and their analogs, metabolites, and derivatives, and beta-carbolines.

In some embodiments, the additional active compound is a phenethylamine. As understood by those in the art, phenethylamines are compounds having the general structure below, wherein R^{N1}, R^{N2}, R^{α}, R^{β}, and each of R²-R⁶ are as taught herein and as generally understood in the art:

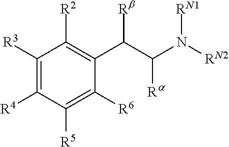

In some embodiments, R^{N1}, R^{N2}, R^{α}, R^{β}, and each of R²-6 are independently hydrogen, deuterium, halogen, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. In some embodiments, R³ and R⁴ are joined together to form an optionally substituted heterocyclyl, such as a dioxole (as with MDMA), a furan, a tetrahydrofuran, a thiophene, a pyrrole, a pyridine, a pyrrolidine, an ethylene oxide, an ethylenimine, a trimethylene oxide, a pyran, a piperidine, an imidazole, a thiazole, a dioxane, a morpholine, or a pyrimidine. In some embodiments, R³ and R⁴ are joined together to form an optionally substituted aryl, such as a phenyl. In some embodiments, the phenethylamine comprises a quaternary ammonium cation wherein each of R^{N1}, R^{N2}, and an additional R^{N3} are independently an alkyl group or an aryl group, and with all other substituents as above. In some embodiments, the phenethylamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, the additional active compound is a phenethylamine selected from the group consisting of α-ethyl-3,4,5-trimethoxy-phenethylamine (AEM), 4-allyloxy-3,5-dimethoxyphenethylamine (AL), 2,5-dimethoxy-4-methylthioamphetamine (ALEPH), 2,5-dimethoxy-4-ethylthioamphetamine (ALEPH-2), 2,5-dimethoxy-4-isopropylthioamphetamine (ALEPH-4), 2,5-dimethoxy-4-phenylthio-amphetamine (ALEPH-6), 2,5-dimethoxy-4-propylthio-amphetamine (ALEPH-7), 2,5-dimethoxy-α-ethyl-4-methylphenethylamine (ARIADNE), 3,4-diethoxy-5-methoxy-phenethylamine (ASB), 4-butoxy-3,5-dimethoxyphenethylamine (B), 2,5-dimethoxy-4,N-dimethylamphetamine (BEATRICE), 2,5-bismethylthio-4-methyl-amphetamine (BIS-TOM), 4-bromo-2,5,ß-trimethoxyphenethylamine (BOB), 2,5,ß-trimethoxy-4-methylphenethylamine (BOD), ß-methoxy-3,4-methylenedioxyphenethylamine (BOH), 2,5-dimethoxy-ß-hydroxy-4-methylphenethylamine (BOHD), 3,4,5,ß-tetramethoxy-phenethylamine (BOM), 4-bromo-3,5-dimethoxyamphetamine (4-Br-3,5-DMA), 2-bromo-4,5-methylenedioxyamphetamine (2-Br-4,5-MDA), 3,4-methylenedioxy-N-ethyl-amphetamine (MDEA), 4-bromo-2,5-dimethoxyphenethylamine (2C-B), 4-benzyloxy-3,5-dimethoxyamphetamine (3C-BZ), 4-chloro-2,5-dimethoxyphenethylamine (2C-C), 2,5-dimethoxy-4-methyl-phenethylamine (2C-D), 2,5-dimethoxy-4-ethyl-phenethylamine (2C-E), 3,5-dimethoxy-4-ethoxyamphetamine (3C-E), 2,5-dimethoxy-4-fluorophenethylamine (2C-F), 2,5-dimethoxy-3,4-dimethylphenethylamine (2C-G), 2,5-dimethoxy-3,4-trimethylene-phenethylamine (2C-G-3), 2,5-dimethoxy-3,4-tetramethylenephenethylamine (2C-G-4), 3,4-norbornyl-2,5-dimethoxyphenethylamine (2C-G-5), 1,4-dimethoxynaphthyl-2-ethylamine (2C-G-N), 2,5-dimethoxyphenethylamine (2C-H), 4-iodo-2,5-dimethoxyphenethylamine (2C-I), 2,5-dimethoxy-4-nitro-phenethylamine (2C-N), 2,5-dimethoxy-4-isopropoxyphenethylamine (2C-O-4), 2,5-dimethoxy-4-propylphenethylamine (2C-P), 4-cyclopropylmethoxy-3,5-dimethoxyphenethylamine (CPM), 2,5-dimethoxy-4-methylselenophenethylamine (2C-SE), 2,5-dimethoxy-4-methylthiophenethylamine (2C-T), 2,5-dimethoxy-4-ethylthiophenethylamine (2C-T-2), 2,5-dimethoxy-4-isopropylthiophenethylamine (2C-T-4), 2,6-dimethoxy-4-isopropylthiophenethylamine (psi-2C-T-4), 2,5-dimethoxy-4-propylthiophenethylamine (2C-T-7), 4-cyclopropylmethylthio-2,5-dimethoxyphenethylamine (2C-T-8), 4-(t)-butylthio-2,5-dimethoxy-phenethylamine (2C-T-9), 2,5-dimethoxy-4-(2-methoxyethylthio) phenethylamine (2C-T-13), 4-cyclopropylthio-2,5-dimethoxyphenethylamine (2C-T-15), 4-(s)-butylthio-2,5-dimethoxy-phenethylamine (2C-T-17), 2,5-dimethoxy-4-(2-fluoroethylthio) phenethylamine (2C-T-21), 3,5-dimethoxy-4-trideuteromethylphenethylamine (4-D), ß,ß-dideutero-3,4,5-trimethoxy-phenethylamine (ß-D), 3,5-dimethoxy-4-methyl-phenethylamine (DESOXY), 2,4-dimethoxy-amphetamine (2,4-DMA), 2,5-dimethoxyamphetamine (2,5-DMA), 3,4-dimethoxyamphetamine (3,4-DMA), 2-(2,5-dimethoxy-4-methylphenyl)cyclopropylamine (DMCPA), 3,4-dimethoxy-ß-hydroxyphenethylamine (DME), 2,5-dimethoxy-3,4-methylenedioxyamphetamine (DMMDA), 2,3-dimethoxy-4,5-methylenedioxyamphetamine (DMMDA-2), 3,4-dimethoxyphenethylamine (DMPEA), 4-amyl-2,5-dimethoxyamphetamine (DOAM), 4-bromo-2,5-dimethoxyamphetamine (DOB), 4-butyl-2,5-dimethoxyamphetamine (DOBU), 4-chloro-2,5-dimethoxyamphetamine (DOC), 2,5-dimethoxy-4-(2-fluoroethyl) amphetamine (DOEF), 2,5-dimethoxy-4-ethyl-amphetamine (DOET), 4-iodo-2,5-dimethoxyamphetamine (DOI), 2,5-dimethoxy-4-methylamphetamine (DOM (STP)), 2,6-dimethoxy-4-methylamphetamine (psi-DOM), 2,5-dimethoxy-4-nitroamphetamine (DON),2,5-dimethoxy-4-propylamphetamine (DOPR), 3,5-dimethoxy-4-ethoxyphenethylamine (E), 2,4,5-triethoxyamphetamine (EEE), 2,4-diethoxy-5-methoxyamphetamine (EEM), 2,5-diethoxy-4-methoxyamphetamine (EME), 4,5-dimethoxy-2-ethoxyamphetamine (EMM), 2-ethylamino-1-(3,4-methylenedioxyphenyl) butane (ETHYL-J), 2-ethylamino-1-(3,4-methylenedioxyphenyl) pentane (ETHYL-K), 6-(2-amino-propyl)-5-methoxy-2-methyl-2,3-dihydrobenzofuran (F-2), 6-(2-aminopropyl)-2,2-dimethyl-5-methoxy-2,3-dihydrobenzofuran (F-22), N-hydroxy-N-methyl-3,4-methylenedioxyamphetamine (FLEA), 2,5-dimethoxy-3,4-(trimethylene) amphetamine (G-3), 2,5-dimethoxy-3,4-(tetra-methylene) amphetamine (G-4), 3,6-dimethoxy-4-(2-aminopropyl)benzonorbornane (G-5), 2,5-dimethoxy-3,4-dimethyl-amphetamine (GANESHA), 1,4-dimethoxynaphthyl-2-isopropyl-amine (G-N), 2,5-dimethoxy-4-ethylthio-N-hydroxyphenethylamine (HOT-2), 2,5-dimethoxy-N-hydroxy-4-(n)-propylthiophenethylamine (HOT-7), 4-(s)-butylthio-2,5-dimethoxy-N-hydroxy-phenethylamine (HOT-17), 2,5-dimethoxy-N,N-dimethyl-4-iodoamphetamine (IDNNA), 2,3,4-trimethoxy-phenethylamine (IM), 3,5-dimethoxy-4-isopropoxyphenethylamine (IP), 5-ethoxy-2-methoxy-4-methylamphetamine (IRIS), 2-amino-1-(3,4-methylenedioxyphenyl) butane (J, BDB), 3-methoxy-4,5-methylenedioxyphenethylamine (LOPHOPHINE), 3,4,5-trimethoxy-phenethylamine (M), 4-methoxyamphetamine (4-MA, PMA), 2,N-dimethyl-4,5-methylenedioxyamphetamine (MADAM-6), 3,5-dimethoxy-4-methallyloxyphenethylamine (MAL), 3,4-methylenedioxyamphetamine (MDA), N-allyl-3,4-methylenedioxyamphetamine (MDAL), N-butyl-3,4-methylenedioxyamphetamine (MDBU), N-benzyl-3,4-methylenedioxy-amphetamine (MDBZ), N-cyclopropylmethyl-3,4-methylenedioxyamphetamine (MDCPM), N,N-dimethyl-3,4-methylenedioxyamphetamine (MDDM), N-ethyl-3,4-methylenedioxy-amphetamine (MDE), N-(2-hydroxyethyl)-3,4-methylenedioxyamphetamine (MDHOET), N-isopropyl-3,4-methylenedioxyamphetamine (MDIP), N-methyl-3,4-methylenedioxy-amphetamine (MDMA), 3,4-ethylenedioxy-N-methylamphetamine (MDMC), N-methoxy-3,4-methylenedioxyamphetamine (MDMEO), N-(2-methoxyethyl)-3,4-methylenedioxyamphetamine (MDMEOET), 3,4-methylenedioxy-α,α,N-trimethylphenethylamine (MDMP), N-hydroxy-3,4-methylenedioxyamphetamine (MDOH), 3,4-methylenedioxyphenethylamine (MDPEA), α,α-dimethyl-3,4-methylenedioxyphenethylamine (MDPH), 3,4-methylenedioxy-N-propargyl-amphetamine (MDPL), 3,4-methylenedioxy-N-propyl-amphetamine (MDPR), 3,4-dimethoxy-5-ethoxyphenethylamine (ME), 4,5-ethylenedioxy-3-methoxyamphetamine (MEDA), 4,5-diethoxy-2-methoxyamphetamine (MEE), 2,5-dimethoxy-4-ethoxyamphetamine (MEM), 4-ethoxy-3-methoxyphenethylamine (MEPEA), 5-bromo-2,4-dimethoxyamphetamine (META-DOB), 2,4-dimethoxy-5-methylthioamphetamine (META-DOT), 2,5-dimethoxy-N-methylamphetamine (METHYL-DMA), 4-bromo-2,5-dimethoxy-N-methylamphetamine (METHYL-DOB), 2-methylamino-1-(3,4-methylenedioxyphenyl) butane (METHYL-J, MBDB), 2-methylamino-1-(3,4-methylenedioxyphenyl) pentane (METHYL-K), 4-methoxy-N-methyl-amphetamine (METHYL-MA, PMMA), 2-methoxy-N-methyl-4,5-methylenedioxyamphetamine (METHYL-MMDA-2), 3-methoxy-4,5-methylenedioxyamphetamine (MMDA), 2-methoxy-4,5-methylenedioxyamphetamine (MMDA-2), 2-methoxy-3,4-methylenedioxyamphetamine (MMDA-3a), 4-methoxy-2,3-methylenedioxyamphetamine (MMDA-3b), 2,4-dimethoxy-5-ethoxyamphetamine (MME), 3,4-dimethoxy-5-(n)-propoxyphenethylamine (MP), 2,5-dimethoxy-4-(n)-propoxyamphetamine (MPM), 4,5-dimethoxy-2-methylthioamphetamine (ORTHO-DOT), 3,5-dimethoxy-4-propoxyphenethylamine (P), 3,5-dimethoxy-4-phenethyloxyphenethylamine (PE), phenethylamine (PEA), 3,5-dimethoxy-4-(2-propynyloxy) phenethylamine (PROPYNYL), 3,5-diethoxy-4-methoxyphenethylamine (SB), 2,3,4,5-tetra-methoxyamphetamine (TA), 4-ethoxy-3-ethylthio-5-methoxyphenethylamine (3-TASB), 3-ethoxy-4-ethylthio-5-methoxyphenethylamine (4-TASB), 3,4-diethoxy-5-methylthio-phenethylamine (5-TASB), 4-(n)-butylthio-3,5-dimethoxyphenethylamine (TB), 4-ethoxy-5-methoxy-3-methylthiophenethylamine (3-TE), 3,5-dimethoxy-4-ethylthiophenethylamine (TE, 4-TE), 3,4-dimethoxy-2-methylthiophenethylamine (2-TIM), 2,4-dimethoxy-3-methylthio-phenethylamine (3-TIM), 2,3-dimethoxy-4-methylthiophenethylamine (4-TIM), 3,4-dimethoxy-5-methylthiophenethylamine (3-TM), 3,5-dimethoxy-4-methylthiophenethylamine (4-TM), 3,4,5-trimethoxyamphetamine (TMA), 2,4,5-trimethoxyamphetamine (TMA-2), 2,3,4-trimethoxyamphetamine (TMA-3), 2,3,5-trimethoxyamphetamine (TMA-4), 2,3,6-trimethoxyamphetamine (TMA-5), 2,4,6-trimethoxyamphetamine (TMA-6), 4,5-dimethoxy-3-ethylthiophenethylamine (3-TME), 3-ethoxy-5-methoxy-4-methylthio-phenethylamine (4-TME), 3-ethoxy-4-methoxy-5-methylthiophenethylamine (5-TME), 3,4-methylenedioxy-2-methylthioamphetamine (2T-MMDA-3a), 2-methoxy-4,5-methylene-thiooxyamphetamine (4T-MMDA-2), 2,4,5-trimethoxyphenethylamine (TMPEA), 4-ethyl-5-methoxy-2-methylthioamphetamine (2-TOET), 4-ethyl-2-methoxy-5-methylthio-amphetamine (5-TOET), 5-methoxy-4-methyl-2-methylthioamphetamine (2-TOM), 2-methoxy-4-methyl-5-methylthioamphetamine (5-TOM), 2-methoxy-4-methyl-5-methyl-sulfinylamphetamine (TOMSO), 3,5-dimethoxy-4-propylthiophenethylamine (TP), 3,4,5-triethoxyphenethylamine (TRIS), 3-ethoxy-5-ethylthio-4-methoxyphenethylamine (3-TSB), 3,5-diethoxy-4-methylthiophenethylamine (4-TSB), 3,4-diethoxy-5-ethylthio-phenethylamine (3-T-TRIS), 3,5-diethoxy-4-ethylthiophenethylamine (4-T-TRIS), (R)-2,5-dimethoxy-4-iodoamphetamine (R-DOI), or a pharmaceutically acceptable salt, hydrate, solvate, prodrug, stereoisomer, or tautomer thereof, or a combination thereof.

In some embodiments, the additional active compound is an ergoline. In some embodiments, the additional active compound is an ergot alkaloid. In some embodiments, the additional active compound is a lysergamide. As understood by those in the art, lysergamides are compounds having the general structure below, wherein R^{N1}, R^{N2}, R¹, R², R⁴, R⁶, R⁷, R⁸, R⁹, R¹², R¹³, and R¹⁴ are as taught herein and as generally understood in the art:

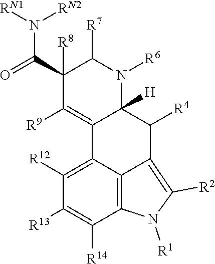

In some embodiments, R^{N1}, R^{N2}, R¹, R², R⁴, R⁶, R⁷, R⁸, R⁹, R¹², R¹³, and R¹⁴ are each independently hydrogen, deuterium, halogen, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. Additionally, any two of R^{N1}, R^{N2}, R¹, R², R⁴, R⁶, R⁷, R⁸, R⁹, R¹², R¹³, and R¹⁴ and the intervening atoms can be taken together to form an optionally substituted optionally substituted cycloalkyl, optionally substituted cycloalkenyl, optionally substituted aryl, or optionally substituted heterocyclyl. In some embodiments, the lysergamide is a quaternary salt, in which an additional R^{6A} is connected to the nitrogen to which R⁶ is bound; wherein R^{6A} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, the additional active compound is a lysergamide selected from the group consisting of lysergic acid diethylamide (i.e., LSD, LSD-25, LAD, Delysid), 6-ethyl-6-nor-lysergic acid diethylamide (ETH-LAD), 6-propynyl-6-nor-lysergic acid diethylamide (PARGY-LAD), 6-allyl-6-nor-lysergic acid diethylamide (AL-LAD), 6-propyl-6-nor-lysergic acid diethylamide (PRO-LAD), 6-isopropyl-6-nor-lysergic acid diethylamide (IP-LAD), 6-cylopropyl-6-nor-lysergic acid diethylamide (CIP-LAD), 6-butyl-6-nor-lysergic acid diethylamide (BU-LAD), 6-(2-fluoroethyl)-6-nor-lysergic acid diethylamide (FLUOROETH-LAD), 1-acetyl-lysergic acid diethylamide (i.e., ALD, ALD-52, N-acetyl-LSD), 1-propionyl-lysergic acid diethylamide (1P-LSD), 1-butyryl-lysergic acid diethylamide (1B-LSD), 1-valeryl-lysergic acid diethylamide (1V-LSD), 1-(cyclopropylmethanoyl)-lysergic acid diethylamide (1 cP-LSD), 1-(1,2-dimethylcyclobutane-1-carbonyl)-lysergic acid diethylamide (1D-LSD), 1-propionyl-6-allyl-6-nor-lysergic acid diethylamide (1P-AL-LAD), 1-(cyclopropylmethanoyl)-6-allyl-6-nor-lysergic acid diethylamide (1 cP-AL-LAD), 1-propionyl-6-ethyl-6-nor-lysergic acid diethylamide (1P-ETH-LAD), lysergic acid 2,4-dimethylazetidide (i.e., LA-SS-Az, LSZ), lysergic acid piperidide (LSD-Pip), and lysergic acid methylisopropyl amide (MIPLA).

Other tryptamines, phenethylamines, and lysergamides useful as additional active compounds for purposes of the invention and thus contemplated for inclusion therein will be as generally known in the art (see, e.g., Shulgin and Shulgin, PiHKAL: A Chemical Love Story, Transform Press (1991); Shulgin and Shulgin, TiHKAL: The Continuation, Transform Press (1997); Grob & Grigsby, Handbook of Medical Hallucinogens, 2021; Luethi & Liechti, Arch. Toxicol., 2020; 94, 1085-1133; Nichols, Pharmacological Reviews, 2016; 68(2), 264-355; Glennon, Pharmacology Biochemistry and Behavior, 1999; 64, 251-256; each of which is incorporated by reference as if fully set forth herein).

### F. Dose and Dosage

In some embodiments, pharmaceutical compositions comprise a therapeutically effective amount or an effective amount of a disclosed compound, such as for administration to a subject. Administration of pharmaceutical compositions in a “therapeutically effective amount,” or an “effective amount” to a subject means administration of an amount of composition sufficient to achieve the desired effect. When an “effective amount” means an amount effective in treating the stated disorder or symptoms in a subject, “therapeutic effect” would be understood to mean the responses(s) in a mammal after treatment that are judged to be desirable and beneficial. Hence, depending on the mental health disorder to be treated, or improvement in mental health or functioning sought, and depending on the particular constituent(s) in the disclosed compositions under consideration, those responses shall differ, but would be readily understood by those of ordinary skill, through an understanding of the disclosure herein and the general knowledge of the art (e.g., by reference to the symptoms listed in the Diagnostic and Statistical Manual of Mental Disorders, 5th Edition (DSM-5) for the stated disorder).

In some embodiments, where a pharmaceutical composition includes a disclosed compound, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition includes a disclosed compound, it may be present in an amount so that a single dose is (in a milligram dosage amount calculated based on the kilogram weight of the patient) between about 0.001 mg/kg and 0.1 mg/kg, such as about 0.001 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, and about 0.1 mg/kg, as well as ranges between these values. In some embodiments, a single dose is between about 0.1 mg/kg and 1.0 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg about 0.8 mg/kg about 0.9 mg/kg, and about 1.0 mg/kg, as well as ranges between these values.

In some embodiments, where a pharmaceutical composition includes a disclosed compound, it may be present in an amount so that a single dose is (in a milligram dosage amount calculated based on the kilogram weight of the patient) about 20 μg/kg body weight or less (e.g., less than 20 μg/kg, less than 15 μg/kg, less than 10 μg/kg, or less than 5 μg/kg body weight, e.g., from 1 to 20 μg/kg body weight, e.g., from 1 to 5 μg/kg, from 5 to 10 μg/kg, from 10 to 15 μg/kg, or from 15 to 20 μg/kg, e.g., about 5 μg/kg, about 10 μg/kg, about 15 μg/kg, or about 20 μg/kg).

In some embodiments, where a pharmaceutical composition includes a disclosed compound, it may be present in an amount so that a single dose is (in a milligram dosage amount calculated based on the kilogram weight of the patient) about less than about 20 ng/ml (e.g., 0.05 to 20 ng/ml, e.g., 0.1 to 15 ng/ml, 0.5 to 10 ng/ml, or 1 to 5 ng/ml, e.g., 0.05 to 0.1 ng/mL, 0.1 to 0.2 ng/ml, 0.2 to 0.3 ng/mL, 0.3 to 0.4 ng/ml, 0.4 to 0.5 ng/mL, 0.5 to 1.0 ng/ml, 1.0 to 5 ng/mL, 5 to 10 ng/mL, 10 to 15 ng/ml, or 15 to 20 ng/ml, e.g., about 0.05 ng/ml, 0.1 ng/ml, 0.2 ng/mL, 0.5 ng/mL, 1.0 ng/ml, 2.0 ng/ml, 2.5 ng/ml, 5.0 ng/ml, 7.5 ng/ml, 10 ng/ml, 12 ng/mL, 15 ng/mL, or 20 ng/mL). In some embodiments, the circulating drug plasma level of the compound is below the limit of detection (e.g., 0.1 ng/ml or less).

In some embodiments, where a pharmaceutical composition includes a disclosed compound, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

In some embodiments, where a pharmaceutical composition includes a disclosed compound, it may be present in an amount so that a single dose is (whether or not such dose is present in a unit dosage form) between about 0.1 mg and 1.0 mg, such as about 0.1 mg, about 0.2 mg, about 0.3 mg, about 0.4 mg, about 0.5 mg, about 0.6 mg, about 0.7 mg, about 0.8 mg, about 0.9 mg, and about 1.0 mg, as well as ranges between these values. In some embodiments, a single dose is between about 1 mg and 10 mg, such as about 1 mg, about 2 mg, about 3 mg, about 4 mg, about 5 mg, about 6 mg, about 7 mg, about 8 mg, about 9 mg, and about 10 mg, as well as ranges between these values. In some embodiments, a single dose is between about 10 mg and 100 mg.

In some embodiments, where a pharmaceutical composition includes an additional active compound, for instance where the additional active compound is a phenethylamine or another tryptamine, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition includes an additional active compound, for instance where the additional active compound is a phenethylamine or a tryptamine, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

It will be appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

Dose amount, frequency or duration may be increased or reduced, as indicated by the clinical outcome desired, status of the pathology or symptom, any adverse side effects of the treatment or therapy, or concomitant medications. One of skill together with the teachings of this disclosure will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

It will be understood that, in some embodiments, the dose actually administered will be determined by a physician, in light of the relevant circumstances, including the disorder to be treated, the chosen route of administration, the actual composition or formulation administered, the age, weight, and response of the individual patient, and the severity of the patient's symptoms, and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, especially where a formulation is prepared in single unit dosage form, such as a capsule, tablet, or lozenge, suggested dosage amounts shall be known by reference to the format of the preparation itself. In other embodiments, where a formulation is prepared in multiple dosage form, for instance liquid suspensions and topical preparations, suggested dosage amounts may be known by reference to the means of administration or by reference to the packaging and labeling, package insert(s), marketing materials, training materials, or other information and knowledge available to those of skill or the public.

Accordingly, another aspect of this disclosure provides pharmaceutical kits containing a pharmaceutical composition or formulation of the invention, suggested administration guidelines or prescribing information therefor, and a suitable container. Individual unit dosage forms can be included in multi-dose kits or containers. Pharmaceutical formulations also can be packaged in single or multiple unit dosage forms for uniformity of dosage and ease of administration.

### G. Kits

Another aspect of this disclosure provides pharmaceutical kits containing a pharmaceutical composition or formulation of the invention, suggested administration guidelines or prescribing information therefor, and a suitable container. Individual unit dosage forms can be included in multi-dose kits or containers. Pharmaceutical formulations also can be packaged in single or multiple unit dosage forms for uniformity of dosage and ease of administration.

Kits generally comprise suitable packaging. The kits may comprise one or more containers comprising any compound described herein. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

Preferably, information pertaining to dosing and proper administration (if needed) will be printed onto a multi-dose kit directly (e.g., on a blister pack or other interior packaging holding the compositions or formulations of the invention); however, kits of the invention can further contain package inserts and other printed instructions (e.g., on exterior packaging) for administering the disclosed compositions and for their appropriate therapeutic use.

### H. Methods of Use

In some aspects, provided herein are methods of using the disclosed compounds. In some embodiments, disclosed compounds are used to modulate neurotransmission. In some embodiments, disclosed compounds are used to treat a condition, such as a disease or a disorder. In some embodiments, disclosed compounds are used in the manufacture of a medicament for the therapeutic and/or the prophylactic treatment of a condition, such as a disease or a disorder. In some embodiments, disclosed compounds are administered as part of therapy. In some embodiments, disclosed compounds are administered along with psychotherapy, psychological support, or patient monitoring. In some embodiments, disclosed compounds are administered in a therapeutically effective amount to a subject having a condition, such as a disease or a disorder. In some embodiments, the condition is a mental health disorder. In some embodiments, the condition is a neurodegenerative disorder. In some embodiments, the condition is a pain disorder. In some embodiments, disclosed compounds are administered to a subject that is healthy.

As used herein, the terms “subject,” “user,” “patient,” and “individual” are used interchangeably, and refer to any mammal, including murines, simians, mammalian farm animals, mammalian sport animals, and mammalian pets, such as canines and felines, although preferably humans. Such terms will be understood to include one who has an indication for which a compound, composition, or method described herein may be efficacious, or who otherwise may benefit by the invention. In general, all of the compounds, compositions, and disclosed methods will be appreciated to work for all individuals, although individual variation is to be expected, and will be understood. The disclosed methods of treatment also can be modified to treat multiple patients at once, including couples or families. Hence, these terms will be understood to also mean two or more individuals.

In some embodiments, disclosed compounds or compositions thereof are orally, mucosally, rectally, subcutaneously, intravenously, intramuscularly, intranasally, by inhalation or transdermally administered to a subject. In some embodiments, when administered through one or more such routes, the disclosed compounds and the disclosed compositions and formulations comprising them are useful in methods for treating a patient in need of such treatment.

a. Modulating Neurotransmission

In some embodiments, disclosed compounds modulate neurotransmission in a subject, such as following administration of a therapeutically effective amount to said subject. In some embodiments, modulating neurotransmission by administering a disclosed compound to a subject treats a disease or disorder in the subject. In some embodiments, modulating neurotransmission comprises regulating levels of monoamines in, for example, the CNS and peripheral tissues. In some embodiments, modulating neurotransmission by administering a disclosed compound to a subject treats a disease or disorder in the subject.

In some embodiments, disclosed compounds activate serotonin receptors. In some embodiments, disclosed compounds agonize and/or antagonize serotonin receptors (5-HT receptors). In some embodiments, disclosed compounds agonize or partially agonize 5-HT receptors, such as any one or more of an 5-HT₁ receptor, such as 5-HT_{1A} and 5-HT_{1B}, an 5-HT₂ receptor, such as 5-HT_{2A}, 5-HT_{2B}, and 5-HT_{2C}, and 5-HT₆.

In some embodiments, a disclosed compound has an in vitro EC₅₀ (agonist mode) for any one or more of 5-HT_{1A}, 5-HT_{1B}, 5-HT_{2A}, 5-HT_{2C}, and 5-HT₆ that is less than 10 μM, less than 5 μM, less than 1 μM, less than 0.5 μM, or less than 0.1 μM. In embodiments, a disclosed compound has an in vitro EC₅₀ (agonist mode) for 5-HT_{2A} that is less than 1 μM, less than 0.5 μM, less than 0.1 μM, less than 0.05 μM, less than 0.01 μM, less than 0.005 μM, or less than 0.001 μM. In embodiments, a disclosed compound has an in vitro EC₅₀ (agonist mode) for 5-HT_{2C} that is less than 1 μM, less than 0.5 μM, less than 0.1 μM, less than 0.05 μM, less than 0.01 μM, less than 0.005 μM, or less than 0.001 μM.

In some embodiments, disclosed compounds show greater potency at 5-HT_{2A} relative to another 5-HT receptor. In some embodiments, disclosed compounds show greater potency at 5-HT_{2A} relative to any one or more of an 5-HT₁ receptor, another 5-HT₂ receptor, such as 5-HT_{2B} and 5-HT_{2C}, a 5-HT₅ receptor, a 5-HT₆ receptor, and a 5-HT₇ receptor.

Determining agonism and antagonism, and measuring EC₅₀ and IC₅₀, respectively, may be determined according to methods available to one of skill in the art. In one example, measuring Gq-mediated calcium flux is a known method for assessing modulation, e.g., activation, of 5-HT_{2A}, a widely recognized target of psychedelic compounds. See, e.g., Klein et al., ACS Pharmacol Transl Sci. 2020 14; 4(2):533-542; Flanagan et al., ACS Pharmacol Transl Sci. 2020; 4(2):488-502; Toro-Sazo et al., PLOS One. 2019; 14(1):e0209804; Halberstadt et al., Psychopharmacology (Berl). 2019; 236(2):799-808. As would be recognized by one of skill, a partial agonist is one that shows reduced maximum efficacy (EMAX) relative to a full agonist (EMAX=100%), e.g., serotonin in the example of a 5-HT receptor.

Because certain therapeutic benefits of disclosed compounds may derive, at least in part, from selective activation of a serotonin receptor (e.g., 5-HT_{2A}, 5-HT_{2C}), one potential approach for improved next-generation compounds with increased therapeutic efficacy, improved safety profiles, and reduced side effects may be optimizing for selective serotonin receptor activation. Accordingly, in some embodiments, a disclosed compound has increased selectivity for the 5-HT_{2A} receptor over another serotonin receptor (e.g., the 5-HT_{2B} receptor, or the 5-HT_{2C} receptor). In some embodiments, a disclosed compound has increased selectivity for the 5-HT_{2A} receptor over the 5-HT_{2B} receptor. In some embodiments, a disclosed compound has increased selectivity for the 5-HT_{2A} receptor over the 5-HT_{2C} receptor. In some embodiments, selectivity is defined as functional activity selectivity, calculated by the ratio of the half-maximal effective concentration (EC₅₀) of a disclosed compound for one receptor (e.g., the 5-HT_{2A} receptor) as compared to another receptor (e.g., a serotonin receptor, such as the 5-HT_{2B} receptor, or the 5-HT_{2C} receptor). For example, if a hypothetical compound had a 5-HT_{2A} EC₅₀ of 0.2 μM and a 5-HT_{2B} EC₅₀ of 1.0 μM, the compound could be said to have a 5-fold functional activity selectivity for the 5-HT_{2A} receptor over the 5-HT_{2B} receptor. In some embodiments, selectivity can be defined as affinity selectivity, defined by the ratio of binding affinity (e.g., as assessed by Kᵢ) for one receptor (e.g., the 5-HT_{2A} receptor) as compared to another receptor (e.g., a serotonin receptor, such as the 5-HT_{2B} receptor, or the 5-HT_{2C} receptor). For example, if a hypothetical compound had a 5-HT_{2A} Kᵢ of 0.1 μM and a 5-HT_{2B} EC₅₀ of 1.0 μM, the compound could be said to have a 10-fold affinity selectivity for the 5-HT_{2A} receptor over the 5-HT_{2B} receptor.

In some embodiments, a disclosed compound has an affinity selectivity of about 1.1-fold, 1.5-fold, 1.6-fold, 2-fold, 5-fold, 10-fold, 20-fold, 30-fold, 50-fold, 70-fold, 80-fold, 90-fold, 100-fold, 150-fold, or at least 150-fold selectivity for the 5-HT_{2A} receptor over the 5-HT_{2B} receptor. In some embodiments, a disclosed compound has improved affinity selectivity for the 5-HT_{2A} receptor over the 5-HT_{2B} receptor, relative to a comparator.

In some embodiments, a disclosed compound has a functional activity selectivity of about 1.1-fold, 1.5-fold, 1.6-fold, 2-fold, 5-fold, 10-fold, 20-fold, 30-fold, 50-fold, 70-fold, 80-fold, 90-fold, 100-fold, 150-fold, or at least 150-fold selectivity for the 5-HT_{2A} receptor over the 5-HT_{2B} receptor. In some embodiments, a disclosed compound has improved affinity selectivity for the 5-HT_{2A} receptor over the 5-HT_{2B} receptor, relative to a comparator.

In some embodiments, a disclosed compound has an affinity selectivity of about 1.1-fold, 1.5-fold, 1.6-fold, 2-fold, 5-fold, 10-fold, 20-fold, 30-fold, 50-fold, 70-fold, 80-fold, 90-fold, 100-fold, 150-fold, or at least 150-fold selectivity for the 5-HT_{2C} receptor over the 5-HT_{2B} receptor. In some embodiments, a disclosed compound has improved affinity selectivity for the 5-HT_{2C} receptor over the 5-HT_{2B} receptor, relative to a comparator.

In some embodiments, a disclosed compound has a functional activity selectivity of about 1.1-fold, 1.5-fold, 1.6-fold, 2-fold, 5-fold, 10-fold, 20-fold, 30-fold, 50-fold, 70-fold, 80-fold, 90-fold, 100-fold, 150-fold, or at least 150-fold selectivity for the 5-HT_{2C} receptor over the 5-HT_{2B} receptor. In some embodiments, a disclosed compound has improved affinity selectivity for the 5-HT_{2C} receptor over the 5-HT_{2B} receptor, relative to a comparator.

b. Modulating Neuroplasticity

In some embodiments, disclosed compounds are used to increase neural plasticity. Neural plasticity, also known as neuroplasticity or brain plasticity, refers to the brain's ability to change and adapt in response to experiences, learning, and environmental factors. Neural plasticity occurs through several mechanisms, including synaptic plasticity, which involves the strengthening or weakening of connections (synapses) between neurons. Synaptic plasticity is often associated with learning and memory processes. Another form of plasticity is called structural plasticity, which involves changes in the physical structure of neurons, such as the growth of new dendritic branches or the formation of new synapses.

Neural plasticity can be defined in terms of neuritogenesis, spinogenesis, and synaptogenesis in neurons. Neuritogenesis refers to the process by which neurons generate and extend their neurites (i.e., to form axons and dendrites). Neuritogenesis is a critical step in neural development and the formation of neuronal circuits. Spinogenesis refers to the formation of dendritic spines, which are small protrusions on the dendrites of neurons. Dendritic spines are crucial for synaptic connections and play a vital role in synaptic transmission and plasticity. Synaptogenesis refers to the formation of synapses, which is crucial for the establishment and refinement of neural circuits, and is a fundamental process underlying learning, memory, and information processing in the brain.

In some embodiments, administration of a disclosed compound to a subject results in an increase in the number of dendritic branches, the number of dendritic crossings, the density of dendritic spines, the density of synapses (i.e., number of synapses per neuron), or total dendritic length. These factors can be measured using a Sholl analysis and other techniques known to those of skill in the art (Ly C, et al. ACS Pharmacology & Translational Science. 2020:4(2); 452-460).

c. Treatment

In some embodiments, disclosed compounds are used to treat a medical condition, such as a disease or disorder. In some embodiments, disclosed compounds are used in the manufacture of a medicament to treat a condition, such as a disease or disorder. Also provided are methods of administering disclosed compounds to a subject having a condition, such as a disease or disorder, thereby treating said condition.

In some embodiments, disclosed compounds or pharmaceutical compositions comprising the disclosed compounds are administered to a subject by one or more routes of administration, including, e.g., oral, mucosal, rectal, subcutaneous, intravenous, intramuscular, intranasal, inhaled, ocular, intraocular, topical, and transdermal routes. When administered through one or more of such routes, the compound(s) of the invention and the disclosed compositions and formulations comprising them are useful in methods for treating a patient in need of such treatment.

In some embodiments are provided methods of treating and/or preventing a condition in a mammal, the method comprising administering to the mammal a therapeutically effective amount of a disclosed compound or pharmaceutical composition. In some embodiments, “treating” or “treatment” refers to treating a disease or disorder in a mammal, and preferably in a human, and includes causing a desired biological or pharmacological effect, such as: (a) preventing a disorder from occurring in a subject who may be predisposed to the disorder but has not yet been diagnosed with it; (b) inhibiting a disorder, i.e. arresting its development; (c) relieving a disorder, i.e., causing regression thereof; (d) protecting from or relieving a symptom or pathology caused by or related to a disorder; (e) reducing, decreasing, inhibiting, ameliorating, or preventing the onset, severity, duration, progression, frequency or probability of one or more symptoms or pathologies associated with a disorder; and (f) preventing or inhibiting of a worsening or progression of symptoms or pathologies associated with a disorder or comorbid with a disorder. In embodiments, treatment includes prevention. In other embodiments, treatment does not include prevention. Other such measurements, benefits, and surrogate or clinical endpoints, alone or in combination, will be understood to one of skill in view of the teachings herein and the knowledge in the art.

In some embodiments, disclosed compounds are used to treat a central nervous system (CNS) disorder. Broadly, CNS disorders include diseases of the nervous system (e.g., movement disorders, neurodegenerative disorders) as well as mental, behavioral, and neurodevelopmental disorders, such as those characterized by the DSM-5, Merck Manual, ICD-11, or other such diagnostic resources known to those of skill.

i. Mental, Behavioral, or Neurodevelopmental Disorders

In some embodiments, disclosed compounds are used to treat a mental, behavioral, or neurodevelopmental disorder. In some embodiments, disclosed compounds are administered, such as in a therapeutically effective amount, to a subject having a mental, behavioral, or neurodevelopmental disorder, thereby treating said mental, behavioral, or neurodevelopmental disorder. In some methods herein, the disclosed compositions, when administered in a therapeutically effective amount, provide beneficial therapeutic effects for the treatment of a mental, behavioral, or neurodevelopmental disorder.

The ICD-11, which is incorporated by reference herein in its entirety, defines “mental, behavioral, or neurodevelopmental disorders” as syndromes characterized by clinically significant disturbance in an individual's cognition, emotional regulation, or behavior that reflects a dysfunction in the psychological, biological, or developmental processes that underlie mental and behavioral functioning. Such disorders include, but are not limited to, neurodevelopmental disorders, schizophrenia or other primary psychotic disorders, catatonia, mood disorders, anxiety or fear-related disorders, obsessive-compulsive or related disorders, disorders specifically associated with stress, dissociative disorders, feeding (or eating) disorders, elimination disorders, disorders of bodily distress or bodily experience, disorders due to substance use or addictive behaviors, impulse control disorders, disruptive behavior or dissocial disorders, personality disorders (and related traits), paraphilic disorders, factitious disorders, neurocognitive disorders, mental or behavioral disorders associated with pregnancy, childbirth or the puerperium, sleep-wake disorders, sexual dysfunctions, and gender incongruence.

A mental, behavioral, or neurodevelopmental disorder where otherwise undefined, will be understood to refer to the disorder as defined in the ICD-11. Within the category of mental, behavioral, or neurodevelopmental disorders, the term mental disorder (or “mental health disorder”) generally refers to a disease condition that involves negative changes in emotion, mood, thinking, and/or behavior. In general, mental health disorders are characterized by clinically significant disturbances in an individual's cognition, emotion, behavior, or a combination thereof, resulting in impaired functioning, distress, or increased risk of suffering. Although the terms “mental disorder” and “mental health disorder,” as well as terms that define specific diseases and disorders, generally shall refer to the criteria in the ICD-11, or a patient with a diagnosis based thereon, it will be appreciated that disclosed methods are equally applicable to patients having an equivalent underlying disorder, whether that disorder is diagnosed based on the criteria in ICD-11, ICD-10, DSM-5, or DSM-IV (each of which is incorporated by reference herein in its entirety) whether the diagnosis is based on other clinically acceptable criteria, or whether the patient has not yet had a formal clinical diagnosis.

In some embodiments, disclosed compounds are used to treat a mental health disorder. In some embodiments, disclosed compounds are administered, such as in a therapeutically effective amount, to a subject having a mental health disorder, thereby treating said mental health disorder. In some methods herein, the disclosed compositions, when administered in a therapeutically effective amount, provide beneficial therapeutic effects for the treatment of a mental health disorder. In some embodiments, the compounds and compositions of the invention are used to reduce the symptoms of a mental health disorder. The symptoms of the mental health disorder to be treated shall be able to be determined by one of skill in the art, by reference to the general understanding of the art regarding that disorder.

In some embodiments, measures of therapeutic efficacy include reports by a subject or an observer. In some embodiments, measures of therapeutic efficacy include responses to a questionnaire. Non-limiting representative examples of applicable measures of symptom improvement include the Generalized Anxiety Disorder Scale-7 (GAD-7), Montgomery-Asberg Depression Rating Scale (MADRS), Global Assessment of Functioning (GAF) Scale, Clinical Global Impression (CGI), Substance Abuse Questionnaire (SAQ), Mini International Neuropsychiatric Interview 5 (MINI 5), Columbia Suicide Severity Rating Scale (C-SSRS), Patient Health Questionnaire (PHQ-9), Pittsburgh Sleep Quality Index (PSQI), Interpersonal Reactivity Index (IRI), Short Form (36) Health Survey (SF-36), Self-Compassion Scale (SCS), Trauma History Questionnaire (THQ), Beck Depression Index (BDI), and related subject- or observer-reported measures.

In some embodiments, a disclosed compound is used to treat a neurodevelopmental disorder. In some embodiments, a “neurodevelopmental disorder” is a neurological and/or cognitive disorder that arises during the developmental period that involves significant difficulties in the acquisition and execution of specific neurological functions (e.g., intellectual, motor, language, or social functions). In some embodiments, the neurodevelopmental disorder is a disorder of intellectual development, a developmental speech or language disorder, autism spectrum disorder, a developmental learning disorder, a developmental motor coordination disorder, attention deficit hyperactivity disorder, or stereotypic movement disorder.

In some embodiments, a disclosed compound is used to treat schizophrenia or another primary psychotic disorder. In general, these disorders are characterized by significant impairments in reality and alterations in behavior manifest in positive symptoms like persistent delusions, persistent hallucinations, disorganized thinking and speech, grossly disorganized behavior, as well as experience of negative symptoms such as blunted or flat affect and avolition and psychomotor disturbances. In some embodiments, a disclosed compound is used to treat schizophrenia, schizoaffective disorder, schizotypal disorder, acute and transient psychotic disorder, delusional disorder, or a substance-induced psychotic disorder.

In some embodiments, a disclosed compound is used to treat catatonia. In some embodiments, “catatonia” refers to a category of syndromes characterized by the co-occurrence of several symptoms of decreased, increased, or abnormal psychomotor activity. In some embodiments, the catatonia is associated with another mental disorder. In some embodiments, the catatonia is induced by substances or medications.

In some embodiments, a disclosed compound is used to treat a mood disorder. As defined in the ICD-11, mood disorders are categorized according to the specific type(s) of mood episodes, and their pattern over time. The primary types of mood episodes are depressive episodes, manic episodes, mixed episodes, and hypomanic episodes. In some embodiments, the mood disorder is a bipolar or related disorder (e.g., bipolar type I disorder, bipolar type II disorder, cyclothymic disorder), a depressive disorder, or a substance-induced mood disorder. In some embodiments, the mood disorder is a depressive disorder. In embodiments, the depressive disorder is single-episode depressive disorder, major depressive episode disorder, persistent depressive disorder (formally known as dysthymia), disruptive mood dysregulation disorder, premenstrual dysphoric disorder, postpartum depression, substance/medication-induced depressive disorder, depressive disorder due to another medical condition, seasonal affective disorder, mixed depressive and anxiety disorder, or an unspecified depressive disorder. In embodiments, depression is assessed through the Patient Health Questionnaire-9 (PHQ-9) screening tool, Montgomery-Åsberg Depression Rating Scale (MADRS), Hamilton Depression Rating Scale, Beck Depression Inventory (BDI-II), Zung Self-Rating Depression Scales (SDS), Major Depression Inventory (MDI), Center for Epidemiologic Studies Depression Scale (CED-D), Rome Depression Inventory (RDI), Hamilton Rating Scale for Depression (HRSD), and Carroll Rating Scale (CRS).

In some embodiments, a disclosed compound is used to treat an anxiety or fear-related disorder. An “anxiety disorder” refers to a class of mental disorders that induce excessive or abnormal fear, dread, or worry. In some embodiments, the anxiety disorder is selected from the group consisting of generalized anxiety disorder, panic disorder, agoraphobia, specific phobia, social anxiety disorder, separation anxiety disorder, selective mutism, or a substance-induced anxiety disorder.

In some embodiments, a disclosed compound is used to treat an obsessive-compulsive or related disorder. In general, these disorders are characterized by repetitive thoughts and behaviors, such as cognitive phenomena (obsessions, intrusive thoughts and preoccupations). In some embodiments, the disorder is characterized by a compulsive need to accumulate possessions and distress related to discarding them (i.e., hoarding disorder). In some embodiments, the disorder is body-focused and can be characterized by recurrent and habitual actions (hair-pulling, skin-picking). In some embodiments, the disorder is obsessive-compulsive disorder, body dysmorphic disorder, olfactory reference disorder, hypochondriasis, hoarding disorder, a body-focused repetitive behavior disorder, or a substance-induced obsessive-compulsive disorder.

In some embodiments, a disclosed compound is used to treat a disorder associated with stress. In some embodiments, the disorder associated with stress has an identifiable stressor that is a causal factor, like exposure to a stressful or traumatic event, or a series of such events or adverse experiences. Stressors may be within the normal range of life experiences (e.g., divorce, socioeconomic problems), or from a threatening or traumatizing experience. In general, the nature and duration of the symptoms that arise in response to the stressor can distinguish the disorder from everyday stress. In some embodiments, a disclosed compound is used to treat post-traumatic stress disorder, complex post-traumatic stress disorder, prolonged grief disorder, adjustment disorder, reactive attachment disorder, or disinhibited social engagement disorder.

In some embodiments, a disclosed compound is used to treat a dissociative disorder. Dissociative disorders can be characterized by involuntary disruption or discontinuity in the normal integration of one or more of the following: identity, sensations, perceptions, affects, thoughts, memories, control over body movements, or behavior. In some subjects, dissociative disorder symptoms can be severe, and may result in impairment in personal, social, educational, occupational or other areas of functioning. In some embodiments, a disclosed compound is used to treat dissociative neurological symptom disorder, dissociative amnesia (including amnesia with dissociative fugue and without dissociative fugue), trance disorder, possession trance disorder, dissociative identity disorder, partial dissociative identity disorder, or depersonalization-derealization disorder.

In some embodiments, a disclosed compound is used to treat a feeding or eating disorder. Feeding or eating disorders generally involve abnormal eating or feeding behaviors that are not explained by another health condition, and are not developmentally appropriate or culturally sanctioned. These disorders can involve preoccupation with food as well as body weight and shape concerns. In some embodiments, a disclosed compound is used to treat anorexia nervosa (including anorexia with significantly low body weight, anorexia with dangerously low body weight, or anorexia in recovery with normal body weight), bulimia nervosa, binge eating disorder, avoidant-restrictive food intake disorder, pica, or rumination-regurgitation disorder.

In some embodiments, a disclosed compound is used to treat an elimination disorder. Elimination disorders include, for example, the repeated voiding of urine into clothes or bed, and the repeated passage of feces in inappropriate places once the individual has reached a developmental age when continence is ordinarily expected. In some embodiments, a disclosed compound is used to treat enuresis (including nocturnal enuresis, diurnal enuresis, and nocturnal and diurnal enuresis) or encopresis (including both with encopresis constipation or overflow incontinence, and encopresis without constipation or overflow incontinence).

In some embodiments, a disclosed compound is used to treat a disorder of bodily distress or bodily experience. Disorders of bodily stress typically involve bodily symptoms that the subject finds distressing and to which the subject devotes excessive attention. Bodily integrity dysphoria typically involves a disturbance in the person's experience of the body manifested by persistent discomfort or intense feelings of body configuration. In some embodiments, a disclosed compound is used to treat a bodily distress disorder (including mild, moderate, and severe bodily distress disorder) or body integrity dysphoria.

In some embodiments, a disclosed compound is used to treat a disorder due to substance use or addictive behaviors. Disorders due to substance use or addictive behaviors are mental and/or behavioral disorders that develop predominantly as a result of the use of psychoactive substances (including medications and illegal or illicit substances), or specific repetitive rewarding and reinforcing behaviors. In some embodiments, a disclosed compound is used to treat disorders due to substance use (i.e., a substance use disorder, or SUD). In some embodiments, the substance use disorder is associated with alcohol, cannabis, synthetic cannabinoids, opioids, sedatives, hypnotics or anxiolytics, cocaine, stimulants (e.g., amphetamines, methamphetamines, methcathinone, synthetic cathinones, caffeine), hallucinogens, nicotine, volatile inhalants, MDMA or MDA, dissociative drugs like ketamine and phencyclidine, or another substance (including medications and non-psychoactive substances). In some embodiments, the substance use disorder is selected from alcohol use disorder, cannabis use disorder, caffeine use disorder, phencyclidine use disorder, inhalants use disorder, opioids use disorder, sedatives use disorder, hypnotics use disorder, anxiolytics use disorder, stimulants use disorder, and tobacco use disorder. In some embodiments, the substance use disorder is alcohol use disorder. In some embodiments, the substance use disorder is cannabis use disorder. In some embodiments, the substance use disorder is caffeine use disorder. In some embodiments, the substance use disorder is phencyclidine use disorder. In some embodiments, the substance use disorder is inhalant use disorder. In some embodiments, the substance use disorder is opioids use disorder. In some embodiments, the substance use disorder is sedatives use disorder. In some embodiments, the substance use disorder is hypnotics use disorder. In some embodiments, the substance use disorder is anxiolytics use disorder. In some embodiments, the substance use disorder is stimulants use disorder. In some embodiments, the substance use disorder is tobacco use disorder. In some embodiments, the substance use disorder is alcohol use disorder, wherein said alcohol use disorder is selected from alcohol abuse, alcohol dependence, and alcoholism. In some embodiments, the disorder is associated with another addictive behavior (e.g., gambling disorders, gaming disorder). In some embodiments, a substance use disorder can be screened using a Screening to Brief Intervention (S2BI), Alcohol, Smoking, and Substance Involvement Screening Test (ASSIST), Brief Screener for Alcohol, Tobacco, and other Drugs (BSTAD), Tobacco, Alcohol, Prescription medication, and other Substance use (TAPS), the Opioid Risk Tool-OUD (ORT-OUD) Chart, Drug Abuse Screen Test (DAST-10), and Tobacco, Alcohol, Prescription medication, and other Substance use (TAPS).

In some embodiments, a disclosed compound is used to treat an impulse control disorder. In general, impulse control disorders are characterized by the repeated failure to resist an impulse, drive, or urge to perform an act that is rewarding to the subject despite negative long-term consequences, such as harm to the subject or a significant impairment in important areas of the subject's functioning. In some embodiments, impulse control behaviors include fire-setting, stealing, inappropriate sexual behavior, and explosive outbursts. In some embodiments, a disclosed compound is used to treat pyromania, kleptomania, compulsive sexual behavior disorder, or intermittent explosive disorder.

In some embodiments, a disclosed compound is used to treat a disruptive behavior disorder or a dissocial disorder. Such disorders may be broadly characterized by persistent behavior problems that range from persistently defiant, disobedient, provocative or spiteful behaviors to behaviors that violate the rights of others or norms, rules, or laws. In some embodiments, a disclosed compound is used to treat oppositional defiant disorder (including oppositional defiant disorder with chronic irritability-anger and oppositional defiant disorder without chronic irritability-anger) or conduct-dissocial disorder (including childhood-onset conduct-dissocial disorder and adolescent-onset conduct-dissocial disorder).

In some embodiments, a disclosed compound is used to treat a personality disorder. Personality disorders may be generally characterized by problems in perceiving one's identity, self-worth, accuracy of self-view, and self-discretion that is manifest in patterns of cognition, emotional experience, emotional expression, and maladaptive behavior. In some embodiments, a disclosed compound is used to treat a mild, moderate, or severe personality disorders. In some embodiments, a disclosed compound is used to treat a prominent personality trait or patterns (e.g., negative affectivity, detachment, dissociality, disinhibition, anankastia, borderline pattern). In some embodiments, the personality disorder is antisocial personality disorder, avoidant personality disorder, borderline personality disorder, dependent personality disorder, histrionic personality disorder, masochistic or sadistic behavior, narcissistic personality disorder, obsessive-compulsive personality disorder, paranoid personality disorder, psychopathy, sociopathy, schizoid personality disorder, or schizotypal personality disorder.

In some embodiments, a disclosed compound is used to treat a paraphilic disorder. Paraphilic disorders can be characterized by persistent and intense patterns of atypical sexual arousal, the focus of which involves others whose age or status renders them unwilling or unable to consent. In some embodiments, a disclosed compound is used to treat exhibitionistic disorder, voyeuristic disorder, pedophilic disorder, coercive sexual sadism disorder, frotteuristic disorder, other paraphilic disorders involving non-consenting individuals, or paraphilic disorders involving solitary behavior or consenting individuals.

In some embodiments, a disclosed compound is used to treat a factitious disorder. In general, factitious disorders may be characterized by intentionally feigning, falsifying, inducing or aggravating medical, psychological, or behavior signs and symptoms or injury to oneself or another person. Subjects with factitious disorders may seek treatment or otherwise present themselves or another person as ill, injured, or impaired. In embodiments, a disclosed compound is used to treat factitious disorder imposed on self or a factitious disorder imposed on another.

In some embodiments, a disclosed compound is used to treat a neurocognitive disorder. Neurocognitive disorders may be characterized by primary clinical defects in cognitive functioning that are acquired (rather than developmental), and therefore the subject experiences a decline from a previously attained level of functioning. In some embodiments, a disclosed compound is used to treat delirium. In some embodiments, the delirium is associated with another disease or disorder. In some embodiments, the delirium is associated with a psychoactive substance (including medications and illicit or illegal substances). In some embodiments, a disclosed compound is used to treat mild neurocognitive disorder. In some embodiments, a disclosed compound is used to treat an amnestic disorder. In some embodiments, the amnestic disorder is associated with another disease or disorder. In some embodiments, the delirium is associated with a psychoactive substance (including medications and illicit or illegal substances). In some embodiments, a disclosed compound is used to treat dementia. In some embodiments, the dementia is associated with Alzheimer's disease, Parkinson's disease, cerebrovascular disease, Lewy body disease, a psychoactive substance (including medications and illicit or illegal substances). In some embodiments, a disclosed compound is used to treat a behavioral or psychological disturbance associated with dementia. In some embodiments, dementia is assessed using a Functional Activities Questionnaire (FAQ), Ascertain Dementia 8 (AD8), Mini-Cog, Mini-Mental State Exam (MMSE), the Montreal Cognitive Assessment (MoCA), and the Neuropsychiatric Inventory Questionnaire (NPI-Q).

In some embodiments, a disclosed compound is used to treat a mental or behavioral disorder associated with pregnancy, childbirth, or the puerperium. In some embodiments, the syndrome associated with pregnancy or the puerperium involves significant mental and behavioral features, including a depressive symptom. In some embodiments, the disorder includes psychotic symptoms. In some embodiments, a disclosed compound is used to treat mental or behavioral disorders associated with pregnancy, childbirth or the puerperium, with psychotic symptoms. In some embodiments, a disclosed compound is used to treat mental or behavioral disorders associated with pregnancy, childbirth or the puerperium, without psychotic symptoms.

In some embodiments, a disclosed compound is used to treat a sleep-wake disorder. In general, sleep-wake disorders are associated with difficulty initiating or maintaining sleep (e.g., insomnia), excessive sleepiness (e.g., hypersomnolence disorders), respiratory disturbance during sleep (e.g., sleep-related breathing disorders (SRBDs), such as obstructive sleep apnea (OSA), central sleep apnea (CSA), sleep-related hypoventilation disorders, sleep-related hypoxemia disorder, snoring, catathrenia, Cheyne-Stokes breathing, and sleep-disordered breathing), disorders of the sleep-wake schedule (e.g., circadian rhythm sleep-wake disorders), abnormal movements during sleep, or problematic behavioral or psychological events that occur while falling asleep, during sleep, or upon arousal from sleep (e.g., parasomnia disorders). In some embodiments, a disclosed compound is used to treat an insomnia disorder, a hypersomnolence disorder, a sleep-related breathing disorder, a circadian rhythm sleep-wake disorder, or a parasomnia disorder.

In some embodiments, a disclosed compound is used to treat sexual dysfunction. Sexual dysfunctions can be defined as syndromes wherein a subject may have difficulty experiencing personally satisfying, non-coercive sexual activities. In some embodiments, a disclosed compound is used to treat hypoactive sexual desire dysfunction, sexual arousal dysfunction, orgasmic dysfunction, ejaculatory dysfunction, or sexual dysfunction associated with pelvic organ prolapse.

In some embodiments, a disclosed compound or composition is administered together with psychotherapy, such as psychosocial or behavioral therapy, including any of (or adapted from any of) cognitive behavioral therapy (e.g., as described in Arch Gen Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J Consul Clin Psychol 2005; 73(2): 354-59; or in Case Reports in Psychiatry, Vol. 2012, Article ID 731638), motivational interviewing based therapy (e.g., as described in J Consul Clin Psychol 2001; 69(5): 858-62), meditation based therapy, such as transcendental meditation based therapy (e.g., as described in J Consul Clin Psychol 2000; 68(3): 515-52), or the therapeutic approach used by MAPS to treat patients with PTSD (e.g., as in Mithoefer, M (2017). Manual for MDMA-Assisted Psychotherapy in the Treatment of Post-traumatic Stress Disorder).

In some embodiments, disclosed compounds and compositions may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention. Furthermore, a patient can participate in numerous other therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it should be understood that such participation can occur with or without the participation or guidance of a therapist.

In some embodiments, “psychotherapy” is specifically “psychedelic-assisted psychotherapy.” Psychedelic-assisted psychotherapy, broadly, includes a range of related approaches that involve at least one session where the patient ingests a psychedelic and is monitored, supported, or otherwise engaged by one or more trained mental health professionals while under the effects of the psychedelic (see, e.g., Schenberg 2018). Protocols have been developed for the standardization of procedures which emphasize a high degree of care (see, e.g., Johnson 2008), such as the therapeutic approach used by MAPS to treat patients with PTSD using MDMA (e.g., as described in Mithoefer 2017).

In some embodiments, the psychotherapy conducted with a disclosed compound is conducted in widely spaced sessions. These sessions can be as frequently as weekly but are more often approximately monthly or less frequently. In most cases, a small number of sessions, on the order of one to three, is needed for a patient to experience significant clinical progress, as indicated, for example, by a reduction in the symptoms of the mental health disorder being treated. In some embodiments, psychotherapy comprises multiple sessions, during some of which a disclosed compound is administered (“drug-assisted psychotherapy”); in others, the patient participates in psychosocial or behavioral therapy without concomitant administration of a drug, or without administration of a disclosed compound.

In some embodiments, a disclosed compound or composition is administered together with standardized psychological treatment or support, which refers to any accepted modality of standard psychotherapy or counseling sessions, whether once a week, twice a week, or as needed; whether in person or virtual (e.g., over telemedicine or by means of a web program or mobile app); and whether with a human therapist or a virtual or AI “therapist.” As used herein, “therapist” refers to a person who treats a patient using the disclosed compositions and methods, whether that person is a psychiatrist, clinical psychologist, clinical therapist, registered therapist, psychotherapist, or other trained clinician, counselor, facilitator, or guide, although it will be understood that certain requirements will be appropriate to certain aspects of the drug-assisted therapy (e.g., prescribing, dispensing, or administering a drug, offering psychotherapeutic support). In some embodiments, a “person” may also include an AI.

In some embodiments, a patient will participate in a treatment protocol or a disclosed method, or be administered a disclosed composition as part of such a method, if the patient meets certain specified inclusion criteria, does not meet certain specified exclusion criteria, does not meet any specified withdrawal criteria during the course of treatment, and otherwise satisfies the requirements of the embodiment of the invention as claimed.

Preferably, where the disclosed pharmaceutical compositions are administered, such administration occurs without or with reduced risk of side effects that would require physician supervision, and therefore allow for treatment at home or otherwise outside of a clinic and without the need for such supervision, and/or additionally without the requirement of adjunctive psychotherapy (although it also may be provided in certain embodiments herein).

In some embodiments, the disclosed compositions may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention. Furthermore, a patient can participate in numerous other therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it should be understood that such participation can occur with or without the participation or guidance of a therapist.

In some instances, certain personalized approaches (i.e., “personalized” or “precision” medicine) may be utilized, based on individual characteristics, including drug metabolism (e.g., CYP2D6 or CYP3A4) or individual genetic variation. The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations.

In one embodiment, the genetic variation is a genetic variation in one or more cytochrome P450 (CYP or CYP450) enzymes that affects drug metabolism, including metabolism of a disclosed composition, and including CYP1A2, CYP2C9, CYP2D6, CYP2C19, CYP3A4 and CYP3A5. Other examples of CYP enzymes include CYP1A1, CYP1B1, CYP2A6, CYP2A13, CYP2B6, CYP2C8, CYP2C9, CYP2C18, CYP2E1, CYP2G1, CYP2J2, CYP2R¹, CYP2S1, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, a disclosed composition is taken together with a compound that is metabolized by the same CYP enzyme(s) as the disclosed composition, so as to permit a lower dose to be taken, increase the effective bioavailability of one or both, or otherwise affect drug metabolism or pharmacokinetics. In some embodiments, the dose of a disclosed composition is adjusted, such as reduced, when administered to a subject known to be a poor metabolizer of an active compound in the composition (e.g., having a genetic variation in CYP2D6 and/or CYP3A4), or increased when administered to a subject known to be a rapid metabolizer. In some embodiments, a patient is tested using ordinary means known to those of skill to determine if the patient is a poor or rapid metabolizer for one or more such CYP enzymes.

In one embodiment, the genetic variation is a genetic variation in metabotropic glutamate receptor type 5 (mGluR5), which has been implicated in mood and anxiety symptoms in humans. In another embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders. In some embodiments, a genetic variation is an inclusion criteria for the administration of a disclosed compound. In some embodiments, a genetic variation is an exclusion criteria for the administration of a disclosed compound.

In one embodiment, the mammal being treated has altered epigenetic regulation of a gene, the expression of which is associated with a mental health condition or susceptibility to a mental health treatment, such as the SIGMAR1 gene for the non-opioid sigma-1 receptor.

ii. Neurodegenerative Disorders

In some embodiments, disclosed compounds are used to treat a neurodegenerative disorder. In some embodiments, disclosed compounds are administered, such as in a therapeutically effective amount, to a subject having a neurodegenerative disorder. In some methods herein, the disclosed compositions, when administered in a therapeutically effective amount, provide beneficial therapeutic effects for the treatment of a neurodegenerative disorder.

The term “neurodegenerative disorder” refers to a class of progressive, chronic, and debilitating conditions characterized by the gradual loss of structure and function of neurons within the central nervous system (CNS) or peripheral nervous system (PNS). These disorders involve the degeneration, impairment, or death of neuronal cells, leading to a decline in cognitive, motor, and/or sensory abilities.

Neurodegenerative disorders can be classified according to primary clinical features, e.g., dementia, parkinsonism, or motor neuron disease, anatomic distribution of neurodegeneration, e.g., frontotemporal degenerations, extrapyramidal disorders, or spinocerebellar degenerations, or principal molecular abnormality (Dugger B, Dickson D W. Pathology of Neurodegenerative Diseases. Cold Spring Harbor Perspectives in Biology. 2017:9(7); a028035). These disorders may involve various etiologies, including but not limited to, presence of pathogenic proteins, age, environmental stressors, and genetic predisposition (Armstrong R. Folia Neuropathologica. 2020:58(2); 93-112).

In some embodiments, the neurodegenerative disorder is selected from the group consisting of Alzheimer's disease, amyotrophic lateral sclerosis or Charcot's disease, chronic traumatic encephalopathy, corticobasal degeneration, dementias including vascular dementia, Huntington's disease, Lytico-Bodig disease, mild cognitive impairment, multiple sclerosis, a motor neuron disease, neuromyelitis optica spectrum disorder, Parkinson's disease or Parkinsonisms, prion diseases, progressive supranuclear palsy, and traumatic brain injury.

iii. Pain Disorders

In some embodiments, disclosed compounds are used to treat a pain disorder. In some embodiments, disclosed compounds are administered, such as in a therapeutically effective amount, to a subject having a pain disorder. In some methods herein, the disclosed compositions, when administered in a therapeutically effective amount, provide beneficial therapeutic effects for the treatment of a pain disorder.

A “pain disorder” refers to a class of medical conditions characterized by the experience of persistent or recurrent physical or psychological pain, either localized or widespread, that significantly impairs an individual's daily functioning and quality of life. These disorders may involve various etiologies, including but not limited to nociceptive, neuropathic, psychogenic, idiopathic or radicular origins. In embodiments, a compound is used to treat neuropathic pain. In embodiments, a compound is used to treat psychogenic pain. In embodiments, a compound is used to treat idiopathic pain. In embodiments, a compound is used to treat radicular pain.

Pain disorders may manifest as acute or chronic pain, and they can affect different parts of the body, such as musculoskeletal, neurological, gastrointestinal, or visceral systems. Pain can be expressed as, but is not limited to, post-herpetic pain, trigeminal pain, occipital pain, or pudendal pain. In embodiments, a disclosed compound is used to treat pain associated with chemotherapy (e.g., chemotherapy associated neuropathy). In embodiments, a disclosed compound is used to treat arthritis, back pain, central pain, chronic fatigue syndrome, cluster headaches, migraine headaches, phantom limb pain, complex regional pain syndrome, compression mononeuropathy, diabetic neuropathy, fibromyalgia, focal neuropathy, herniated disc pain, or sciatica.

In some embodiments, pain is assessed using the Pain, Enjoyment, and General Activity Scale (PEG), the Numeric Rating Scale (NRS), the Visual Analog Scale (VAS), Behavioral Pain Scale (BPS), and the Faces Pain Scale-Revised (FPS-R).

iv. Inflammatory Disorders

Inflammation is an essential immune response to tissue insults such as microbial infection, acute injury, chemical irritants or other such dysregulation of normal tissue functioning. The inflammatory process is a feature of the innate immune system, whereby molecular patterns of tissue damage are recognized and responded to by a variety of inflammatory agents such as cytokines and chemokines. These inflammatory agents act directly to remove harmful stimuli and initiate various signaling responses to return damaged tissue to a state of homeostasis. Although this response is often self-terminating, the resolution of inflammation may fail for multiple reasons, extending the inflammation response into a chronic stage (Ahmed A U. Front Biol. 2011:6(4): 274-281). Chronic inflammation is often associated with or underlies a variety of pathological conditions, including major cardiovascular and neuropsychiatric disorders (Nichols C D. Cardiovasc Psychiatry Neurol 2009:475108).

Recent evidence suggests a significant role of the 5-HT_{2A} serotonin receptor subtype in mediating the termination of the inflammatory response. 5-HT_{2A} receptors are found throughout the body, including in both the central nervous system and peripheral tissues (Flanagan & Nichols. In'l Rev of Psychiatry. 2018. 30(4), 363-375). In the brain, 5-HT_{2A} receptors are involved in cognitive function and working memory, mediate the effects of psychedelic compounds, and have been implicated in mechanisms underlying neuropsychiatric disorders such as schizophrenia (Nichols C D. Cardiovasc Psychiatry Neurol. 2009:475108). In the periphery, 5-HT_{2A} receptors are found in multiple immune related tissues such as the spleen, thymus, and circulating lymphocytes, as well as in components of both the innate and adaptive immune systems (Stefulj J, et al. Brain Behav Immun. 2000 September; 14(3):219-24; Cloëz-Tayarani I, et al. Int Immunol. 2003 February; 15(2):233-40). Research on 5-HT_{2A} receptors at these tissues have elucidated their role in modulating the immune response (Flanagan T W, Nichols C D. Int Rev Psychiatry. 2018 August; 30(4):363-375).

Due to their significant action on 5-HT_{2A} receptors in the brain, multiple studies have been performed to assess the effect of psychedelic compounds on the inflammation modulating effects of 5-HT_{2A} receptors. One such study found that (R)-2,4-dimethoxy-4-iodoamphetamine ((R)-DOI) is able to potently repress TNF-α induced inflammation. This study found the same effect, albeit slightly less potent, induced by the psychedelic compounds 2C-BCB, LA-SS-Az and LSD (Yu et al. J Pharmacol Exp Ther. 2008. 327, 316-323). Notably, the potency required to achieve anti-inflammatory effects of some psychedelic compounds is at levels in the low picomolar range, approximately 500× more potent than conventional corticosteroids at their target. Further, anti-inflammatory doses of psychedelics can be below the threshold for producing subjective or behavioral effects, meaning they may exhibit anti-inflammatory effects without triggering a psychedelic “trip.”

This work, and subsequent in vitro and in vivo studies have demonstrated that (R)-DOI inhibits TNF-α induced expression of genes encoding intracellular adhesion molecule-1 (ICAM1), vascular cell adhesion molecule-1 (VCAM1), and inflammatory cytokines IL-6 and IL-1B, and chemokines monocyte chemotactic protein-1 (MCP1). (R)-DOI also blocks activation and nuclear translocation of NF-\hat{k}B, and nitric oxide synthase activity (Nau F Jr, et al. PLOS One. 2013 Oct. 2; 8(10):e75426; Flanagan & Nichols. Int'l Review Psych. 2018. 30(4), 363-375). Further, some psychedelic compounds potently suppress select key proinflammatory biomarkers, while leaving others unaffected. For the biomarkers where suppression is evident, suppression is potent and returns levels to baseline, not suppressed below baseline levels, even at relatively high doses of drug (Nichols C D. Neuropharmacol. 2022 Nov. 15; 219:109232). Thus, some psychedelics can reduce expression of certain key inflammatory components, while leaving the immune response largely intact. This is a unique mechanism of action among known anti-inflammatory and immunomodulatory agents, and may be advantageous as it is predicted to have fewer side effects such as opportunistic infections that are associated with broad immunosuppressants like corticosteroids (Nichols C D. Neuropharmacol. 2022 Nov. 15; 219:109232).

Although there is great therapeutic potential for psychedelics as anti-inflammatory agents, there is considerable variation in the efficacy of different psychedelics. It has been hypothesized that chemical structural diversity among psychedelics may result in functional selectivity at the 5-HT_{2A} receptor, whereby certain ligands engage specific subsets of amino acid residues in the binding pocket of the receptor that induce stable conformational states that couple to different anti-inflammatory signal transduction affectors. In recent research, the primary pharmacophore for anti-inflammatory phenethylamine 5-HT_{2A} receptor agonists was found to be 2,5-dimethoxyphenethylamine (2C-H) (Flanagan T W, et al. ACS Pharmacol Transl Sci. 2020 Aug. 13; 4(2):488-502). Hence, without being bound by theory, variations of this core pharmacophore may be leveraged to create new compounds with specific anti-inflammatory effects. Further, such compounds may be engineered to elicit variable efficacies at specific anti-inflammatory pathway targets, further increasing their potential to treat chronic inflammatory disorders.

In some embodiments, a disclosed compound is a potent anti-inflammatory agent that acts on specific inflammation mediators, thereby returning chronically inflamed tissue to a healthy state. In some embodiments, the anti-inflammatory effect is enacted without broadly suppressing the immune system, and can therefore be beneficial to treat inflammatory disease where steroids are contraindicated, or the condition is steroid resistant.

In some embodiments, a disclosed compound decreases an inflammatory response in a subject. In some embodiments, the inflammatory response is quantified by a change in the level of an inflammation response biomarker. In some embodiments, the level of an inflammation response biomarker represents the expression level of an inflammation response gene. For example, an increased level of an inflammation response biomarker in a subject can be compared to a baseline level of the same biomarker, said increase being indicative of increased expression of the inflammation response gene encoding that biomarker. In some embodiments, increased expression of an inflammation response gene can be associated with chronic inflammation. In some embodiments, decreased expression of an inflammation response gene can be associated with chronic inflammation.

In some embodiments, a disclosed compound exhibits potent anti-inflammatory properties. In some embodiments, administration of a disclosed compound suppresses several pro-inflammatory markers (e.g., mRNA encoding IL6, IL1b, GMCSF, and IL5). In some embodiments, administration of a disclosed compound suppresses pro-inflammatory markers to baseline levels. Without being bound by theory, disclosed compounds may exert their anti-inflammatory effects due to functional selectivity at the 5-HT_{2A} receptor, whereby the compound engages certain amino acid residues within receptor, stabilizing it in a conformation that triggers anti-inflammatory signal transduction pathway effectors.

In some embodiments, the biomarker of inflammation response gene expression is mRNA. In some embodiments, the biomarker of inflammation response gene expression is a protein. In some embodiments, the inflammation response gene is TNFα, IL-4, IL-5, IL-6, IL-8, IL-9, IL-1B, Il-lA, IL-12, IFNα, IFNb, IFNg, TGF-8, IL-15, IL-17, IL-20, IL-22, LTA, IL-23, IL-18, VCAM1, ICAM1, MCP1, MMP-9, Muc5ac, Gm-csf, CCL2, CCL5, CCL3, CCL4, CCL11, CD11a, CD3, CD4, CD8, or CRP. In some embodiments, the inflammation response gene encodes an inflammatory agent. An inflammatory agent is a protein that activates an inflammatory response. Inflammatory agents include, for example, the proteins IL-1B, TNFα, IL-15, IL-17, and IL-18. In some embodiments, the inflammation response gene encodes an anti-inflammatory agent. An anti-inflammatory agent is a protein that reduces an inflammatory response. Anti-inflammatory agents include, for example, the proteins IL-1, IL-4, IL-10, IL-11, and IL-13. In some embodiments, the inflammation response gene encodes an agent that may be inflammatory or anti-inflammatory. For example, leukemia inhibitory factor, interferon-alpha, IL-6, and transforming growth factor (TGF-β) can act as either inflammatory or anti-inflammatory cytokines under various circumstances (Zhang J M, An J. Int Anesthesiol Clin. 2007 Spring; 45(2):27-37).

In some embodiments, the inflammation response gene is ICAM1. In some embodiments, the biomarker of inflammation response is an ICAM1 gene product. In some embodiments, the biomarker is ICAM1 mRNA. In some embodiments, the biomarker is the ICAM1 protein. In some embodiments, the inflammation response gene is VCAM1. In some embodiments, the biomarker of inflammation response is a VCAM1 gene product. In some embodiments, the biomarker is VCAM1 mRNA. In some embodiments, the biomarker is the VCAM1 protein. In some embodiments, the inflammation response gene is MCP1. In some embodiments, the biomarker of inflammation response is a MCP1 gene product. In some embodiments, the biomarker is MCP1 mRNA. In some embodiments, the biomarker is the MCP1 protein. In some embodiments, the inflammation response gene is IL-5. In some embodiments, the biomarker of inflammation response is a IL-5 gene product. In some embodiments, the biomarker is IL-5 mRNA. In some embodiments, the biomarker is the IL-5 protein. In some embodiments, the inflammation response gene is IL-6. In some embodiments, the biomarker of inflammation response is a IL-6 gene product. In some embodiments, the biomarker is IL-6 mRNA. In embodiments, the biomarker is the IL-6 protein.

In some embodiments, the inflammation response gene is IL-9. In some embodiments, the biomarker of inflammation response is a IL-9 gene product. In some embodiments, the biomarker is IL-9 mRNA. In some embodiments, the biomarker is the IL-9 protein. In some embodiments, the inflammation response gene is IL-15. In some embodiments, the biomarker of inflammation response is a IL-15 gene product. In some embodiments, the biomarker is IL-15 mRNA. In some embodiments, the biomarker is the IL-15 protein. In some embodiments, the inflammation response gene is IL-18. In some embodiments, the biomarker of inflammation response is a IL-1B gene product. In some embodiments, the biomarker is IL-18 mRNA. In some embodiments, the biomarker is the IL-1ß protein.

In some embodiments, the inflammation response gene is Gm-csf. In some embodiments, the biomarker of inflammation response is a Gm-csf gene product. In some embodiments, the biomarker is Gm-csf mRNA. In some embodiments, the biomarker is the Gm-csf protein. In some embodiments, the inflammation response gene is Muc5ac. In some embodiments, the biomarker of inflammation response is a Muc5ac gene product. In some embodiments, the biomarker is Muc5ac mRNA. In some embodiments, the biomarker is the Muc5ac protein. In some embodiments, the inflammation response gene is MMP-9. In some embodiments, the biomarker of inflammation response is a MMP-9 gene product. In some embodiments, the biomarker is MMP-9 mRNA. In some embodiments, the biomarker is the MMP-9 protein. In some embodiments, the inflammation response gene is TGF-8. In some embodiments, the biomarker of inflammation response is a TGF-β gene product. In some embodiments, the biomarker is TGF-β mRNA. In some embodiments, the biomarker is the TGF-β protein.

In some embodiments, the inflammation response biomarker is a cytokine. Cytokines are small signaling proteins that coordinate the interactions of different cell types involved in the amplification and regulation of the inflammatory response. In some embodiments, the cytokine biomarker is IL-2, IFN-γ, TNFα, TNFβ, GM-CSF, IL-2, IL-3, IL-4, IL-5, IL-6, IL-9, IL-10, IL-13, IL-17, IL-25, IL-33, or TGF-β. In some embodiments, the inflammation response biomarker is a chemokine. Chemokines are small signaling proteins that induce the movement of other cell types, such as toward a tissue injury site. In some embodiments, the chemokine biomarker is CCL-1 to CCL-28, CXCL-1 to CXCL-16, IL-8, MCP1, RANTES, XCL1, XCL2, or CX3CL1. In some embodiments, the biomarker of inflammation for a particular inflammatory disease, comorbidity, or patient demographic will be known to those of skill in the art (See: Sreedhar R, et al. General Mechanisms of Immunity and Inflammation. In: Watanabe K & Arumugam S. eds. Japanese Kampo medicines for the treatment of common diseases: Focus on inflammation. Academic Press; 2017: Chapter 3; Germolec D R et al. Markers of Inflammation. Methods Mol Biol. 2018; 1803:57-79; Calder P C, et al. Br J Nutr. 2013 January; 109 Suppl 1:S1-34).

In some embodiments, a disclosed compound causes the level of an inflammation response biomarker in a subject to become closer to a baseline level. “Baseline level” refers to the level of a biomarker observed in healthy populations not experiencing inflammation. Baseline levels differ among biomarkers and will be known to those of skill, or can be measured by standard techniques (Calder P C, et al. Br J Nutr. 2013 January; 109 Suppl 1:S1-34).

In some embodiments, a disclosed compound reduces the level of an inflammatory biomarker. In some embodiments, a disclosed compound does not reduce the level of an inflammatory biomarker below baseline. In some embodiments, a disclosed compound reduces the level of an inflammatory biomarker (e.g., an mRNA biomarker, a cytokine biomarker, a chemokine biomarker) by about 1%, 5%, 10%, 20%, 30%, 40%, 50%, 60%, 70%, 80%, 90%, 95%, or 100%. In some embodiments, a disclosed compound reduces the level of an inflammatory biomarker (e.g., an mRNA biomarker, a cytokine biomarker, a chemokine biomarker) to within about 1%, 5%, 10%, 20%, 30%, 40%, 50%, 60%, 70%, 80%, 90%, 95%, or 100% of its baseline level. In some embodiments, a disclosed compound decreases the concentration of one or more inflammatory biomarkers in a sample by about 100 pg/mL, 90 pg/mL, 80 pg/mL, 70 pg/mL, 60 pg/mL, 50 pg/mL, 40 pg/mL, 30 pg/mL, 20 pg/mL, 10 pg/mL, 5 pg/mL, or 1 pg/mL. In some embodiments, the sample is a tissue sample. In some embodiments, the sample is a blood sample. In some embodiments, the same is a plasma sample.

In some embodiments, a disclosed compound increases the level of an anti-inflammatory biomarker. In some embodiments, a disclosed compound does not increase the level of a pro-inflammation biomarker above baseline. In some embodiments, a disclosed compound increases the level of a pro-inflammation biomarker (e.g., an mRNA biomarker, a cytokine biomarker, a chemokine biomarker) by about 1%, 5%, 10%, 20%, 30%, 40%, 50%, 60%, 70%, 80%, 90%, 95%, or 100%. In some embodiments, a disclosed compound increases the concentration of one or more anti-inflammatory biomarkers in a sample by about 100 pg/mL, 90 pg/mL, 80 pg/mL, 70 pg/mL, 60 pg/mL, 50 pg/mL, 40 pg/mL, 30 pg/mL, 20 pg/mL, 10 pg/mL, 5 pg/mL, or 1 pg/mL. In some embodiments, the sample is a tissue sample. In some embodiments, the sample is a blood sample. In some embodiments, the same is a plasma sample.

In some embodiments, the dosage of a disclosed compound used to elicit an anti-inflammatory effect is sub-behavioral. In some embodiments, a disclosed compound is used to elicit an anti-inflammatory effect at dosage between about 0.001 and 0.01 mg/kg, between about 0.01 and 0.05 mg/kg, between about 0.05 mg/kg and 0.1 mg/kg, between about 0.1 mg/kg and 0.2 mg/kg, between about 0.4 mg/kg and 0.3 mg/kg, between about 0.3 mg/kg and 0.4 mg/kg, or between about 0.4 mg/kg and 0.5 mg/kg.

In some embodiments, a disclosed compound may be used to treat an inflammatory disorder. In some embodiments, a disclosed compound is useful for reducing inflammation. In some embodiments, a disclosed compound is used in the manufacture of a medicament to treat an inflammatory disorder or reduce inflammation.

In some embodiments, the disorder is an acute inflammatory disorder. In some embodiments, the disorder is a chronic inflammatory disorder. In some embodiments, the inflammatory disorder is asthma, chronic obstructive pulmonary disease, neuroinflammation, rheumatoid arthritis, atherosclerosis, psoriasis, type II diabetes, inflammatory bowel disease, Crohn's disease, multiple sclerosis, septicemia, conjunctivitis, Alzheimer's disease, or another inflammatory condition described herein.

In some embodiments, a disclosed compound is useful for treating an inflammatory condition in patients with autoimmune disorders or otherwise compromised immune systems. For example, a disclosed compound is useful for treating chronic inflammation in patients with type 1 diabetes, type 2 diabetes, multiple sclerosis (MS), lupus, rheumatoid arthritis, psoriatic arthritis, reactive arthritis, Addison disease, Celiac disease, autoimmune encephalitis, gout, vasculitis, mixed connective tissue disease, undifferentiated connective tissue disease, myositis, scleroderma, Sjogren's syndrome, uveitis, inflammatory bowel disease (IBD), Guillain-Barre syndrome, psoriasis, grave's disease, scleroderma (systemic sclerosis), dermatomyositis, Hashimoto thyroiditis, pernicious anemia, Alzheimer's disease, heart disease, cardiovascular disease, chronic hepatic and renal disease, fibromyalgia, allergies, or chronic obstructive pulmonary disease (COPD). In some embodiments, a disclosed compound is useful for treating chronic inflammation in an immunocompromised chemotherapy patient.

In some embodiments, a disclosed compound is useful for treating an inflammatory condition in patients with a steroid-resistant disease or disorder. In some embodiments, the steroid-resistant disease or disorder is steroid resistant nephrotic syndrome (SRNS), steroid-resistant inflammatory bowel syndrome (IBS), steroid-resistant asthma, steroid-resistant acute graft-versus-host disease, steroid-resistant ulcerative colitis, steroid-resistant Crohn's disease, steroid-resistant chronic obstructive pulmonary disease (COPD), steroid-resistant pulmonary fibrosis, steroid-resistant leukemias, steroid-resistant rheumatoid arthritis, or steroid-resistant idiopathic nephrosis.

In some embodiments, a disclosed compound is useful for treating an inflammatory condition in a patient with a contraindication to a corticosteroid. Contraindications to corticosteroids can occur, for example, because of hypersensitivity to any component of a corticosteroid formulation, concurrent administration of live or live-attenuated vaccines (e.g., when using immunosuppressive doses), systemic fungal infection, osteoporosis, uncontrolled hyperglycemia, adrenal suppression, Cushing syndrome, diabetes mellitus, glaucoma, cataracts, joint infection, uncontrolled hypertension, herpes simplex keratitis, myopathy, certain psychiatric disturbances and/or disorders, and varicella infection. Additional exemplary contraindications include peptic ulcer disease, congestive heart failure, and viral or bacterial infections not controlled by anti-infective or antibacterial agents.

In some embodiments, a disclosed compound is useful for treating skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, ocular inflammation, or brain inflammation.

In some embodiments, the inflammatory disorder is any of acne vulgaris, oxalic acid/heartburn, age-related macular degeneration (AMD), allergies, allergic rhinitis, Alzheimer's disease, amyotrophic lateral sclerosis, Anemia, appendicitis, arteritis, arthritis, including osteoarthritis, rheumatoid arthritis, juvenile idiopathic arthritis, spondyloarthropathy such as ankylosing spondylitis, reactive arthritis (Reiter syndrome), psoriatic arthritis, enteroarthritis associated with inflammatory bowel disease, Whipple and Behcet's disease, septic arthritis, gout (also known as gouty arthritis, crystalline synovitis, metabolic arthritis), pseudogout (calcium pyrophosphate deposition disease), and Still's disease. Arthritis can affect a single joint (monoarthritis), two to four joints (oligoarthritis), or five or more joints (polyarthritis).

In some embodiments, the inflammatory disorder is any of long COVID, a food allergy, post-treatment lyme disease syndrome, and an ulcer. In some embodiments, an inflammatory disorder is any of asthma, atherosclerosis, autoimmune disorder, balanitis, blepharitis, bronchiolitis, bronchitis, bullous pemphigoid, burns, bursitis, cancer, including NF-κB-induced inflammatory cancer; cardiovascular disease, including hypertension, endocarditis, myocarditis, heart valve dysfunction, congestive heart failure, myocardial infarction, diabetic heart abnormalities, vascular inflammation, including arteritis, phlebitis, and vasculitis; arterial occlusive disease, including arteriosclerosis and stenosis; inflammatory cardiac hypertrophy, peripheral arterial disease, aneurysm, embolism, incision, pseudoaneurysm, vascular malformation, vascular nevus, thrombosis, thrombophlebitis, varicose veins, stroke, cardiac arrest, and carditis; celiac disease, cellulitis, cervicitis, cholangitis, cholecystitis, chorioamnionitis, chronic obstructive pulmonary disease (COPD), cirrhosis, congestive heart failure, conjunctivitis, colitis, cyclophosphamide-induced cystitis, cystic fibrosis, cystitis, lacrimal inflammation, and dementia.

In some embodiments, the inflammatory disorder is a dermatitis disorder. Without being bound by theory, dermatitis refers to inflammation of the skin which can occur chronically due to skin barrier dysfunction, abnormal inflammatory response, and persistent itching (Nakahara T, et al. J Dermatol. 2021; 48(2):130-139; Beck L A, et al. JID Innov. 2022; 2(5):100131). Whereas common among dermatitis disorders include redness, persistent itching, and dry skin, further clinical phenotypes of dermatitis disorders are highly heterogeneous, reflecting the diversity and complexity of the underlying mechanisms leading to the disorder (Renert-Yuval Y, et al. J Allergy Clin Immunol. 2021; 147(4):1174-1190.e1). Many of the inflammatory agents involved in chronic inflammation are also involved in the inflammatory response to dermatitis disorders, including but not limited to CCL17, CCL18, CCL22, CCL27, IL-4, IL-13, IL-17A, IL-18, IL-19, IL-22, IL-26, IL-33, MMP12 (Ahn K, et al. Curr Opin Immunol. 2020; 66:14-21; Renert-Yuval Y, et al. J Allergy Clin Immunol. 2021; 147(4):1174-1190.e1; Furue M, et al. Iran J Immunol. 2019; 16(2):97-107; Sroka-Tomaszewska J, Trzeciak M. Int J Mol Sci. 2021; 22(8):4130). Effective treatments of dermatitis disorders often target inflammatory pathways, thereby regulating the inflammatory response and ameliorating the symptoms of the dermatitis disorder (Wollenberg A, et al. Br J Dermatol. 2014; 170 Suppl 1:7-11).

In some embodiments, the inflammatory disorder is a dermatitis disorder, including atopic dermatitis, chronic photosensitivity dermatitis, eczema, atopic eczema, contact eczema, dryness eczema, seborrheic eczema, discoid eczema, varicose eczema, herpetic dermatitis, neurodermatitis, autosensitizing dermatitis, stasis dermatitis, purulent dermatitis, dyshidrotic eczema, follicular eczema, spongiotic dermatitis, hand dermatitis, diaper dermatitis, occupational contact dermatitis, and lichen planus-like atopic dermatitis.

In some embodiments, the dermatitis disorder is atopic dermatitis. In some embodiments, the dermatitis disorder is chronic photosensitivity dermatitis. In some embodiments, the dermatitis disorder is eczema. In some embodiments, the dermatitis disorder is atopic eczema. In some embodiments, the dermatitis disorder is contact eczema. In some embodiments, the dermatitis disorder is dryness eczema. In some embodiments, the dermatitis disorder is seborrheic eczema. In some embodiments, the dermatitis disorder is discoid eczema. In some embodiments, the dermatitis disorder is varicose eczema. In some embodiments, the dermatitis disorder is herpetic dermatitis. In some embodiments, the dermatitis disorder is neurodermatitis. In some embodiments, the dermatitis disorder is herpetic dermatitis. In some embodiments, the dermatitis disorder is autosensitizing dermatitis. In some embodiments, the dermatitis disorder is stasis dermatitis. In some embodiments, the dermatitis disorder is purulent dermatitis. In some embodiments, the dermatitis disorder is dyshidrotic eczema. In some embodiments, the dermatitis disorder is follicular eczema. In some embodiments, the dermatitis disorder is spongiotic dermatitis. In some embodiments, the dermatitis disorder is hand dermatitis. In some embodiments, the dermatitis disorder is diaper dermatitis. In some embodiments, the dermatitis disorder is occupational contact dermatitis. In some embodiments, the dermatitis disorder is lichen planus-like atopic dermatitis.

In some embodiments, the inflammatory disorder is any of dermatitis, including atopic dermatitis, chronic photosensitivity dermatitis, eczema, atopic eczema, contact eczema, dryness eczema, seborrheic eczema, sweating disorders, discoid eczema, venous eczema, herpetic dermatitis, neurodermatitis, and autosensitizing dermatitis, stasis dermatitis, purulent sweaty, lichen planus, psoriasis, including psoriasis vulgaris, nail psoriasis, prickly psoriasis, scalp psoriasis, inverse psoriasis, pustular psoriasis, erythrodermic psoriasis, and psoriatic arthritis; rosacea, and scleroderma, including morphea; pharmacologically induced inflammation, including from legal or illegal drugs, and chemicals; chronic neurogenic inflammation, including primary and secondary neural inflammation; dermatomyositis, diabetes, diabetic neuropathy, diabetic retinopathy, diabetic nephropathy, diabetic ulcer, digestive system disease, emphysema, encephalitis, endocarditis, endometritis, enterocolitis, epicondylitis, epididymis, fasciitis, fibromyalgia, fibrosis, connectitis, gastritis, gastroenteritis, gingivitis, glomerulonephritis, glossitis, heart disease, heart valvular dysfunction, hepatitis, purulent spondylitis, Huntington's disease, hyperlipidemic pancreatitis, hypertension, ileitis, infection, including lymphangitis, lymphadenitis, bacterial cystitis, bacterial encephalitis, pandemic influenza, viral encephalitis, and viral hepatitis (types A, B, and C); inflammatory bowel disease, including Crohn's disease; inflammatory heart enlargement, inflammatory neuropathy, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), migraine, multiple sclerosis, myelitis, myocarditis, myositis, nephritis, non-alcoholic steatohepatitis, obesity, umbilitis, ovitis, testitis, osteochondritis, osteopenia, osteomyelitis, osteoporosis, osteomyelitis, otitis, pancreatitis, Parkinson's disease, parotitis, pelvic inflammatory disease, pemphigus vulgaris, pericarditis, Peritonitis, pharyngitis, phlebitis, pleurisy, interstitial pneumonia, polycystic nephritis, polymyositis, proctitis, prostatitis, psoriasis, pulpitis, pyelonephritis, portal vein, renal failure, reperfusion injury, retinitis, rheumatic fever Rhinitis, fallopianitis, sarcoidosis, salivary glanditis, sepsis, including bacteremia and viremia; sinusitis, spastic colon, stenosis, stomatitis, stroke, inflammation associated with surgical complications, synovitis, tendonitis, tendonitis, tendonitis, thrombophlebitis, tonsillitis, trauma, traumatic brain injury, graft rejection, including graft versus host disease (GVHD); a Th1-mediated inflammatory disease, trigonitis, tuberculosis, tumor, urethritis, bursitis, uveitis, vaginitis, vasculitis, including Buerger's disease, cerebral vasculitis, Churg-Strauss arteritis, cryoglobulinemia, essential cryoglobulin vasculitis, giant cells arteritis, golfer vasculitis, Henoch-Schönlein purpura, hypersensitivity vasculitis, Kawasaki disease, microscopic polyarteritis/polyvasculitis, nodular polyarteritis, rheumatoid polymuscular muscle pain (PMR), rheumatic vasculitis, Takayasu arteritis, Wegener's granulomatosis, systemic lupus erythematosus (SLE), relapsing polychondritis, Behcet's disease; ulcerative colitis such as ulcerative proctitis, left side colitis, total colitis, and fulminant colitis; and vulvitis.

In some embodiments, a disclosed compound is used to treat an ophthalmic disease or disorder. Ophthalmic diseases and disorders often result from infection and/or inflammation of ocular tissue, and are the leading cause of corneal blindness and visual morbidity worldwide (Bourne R R, et al. Lancet Glob Health. 2013; 1(6):e339-49). Repeated episodes of either infection or inflammation triggers a chronic inflammatory disease process that can result in vascularization and subsequent vision threatening scarring of the cornea (Vaidyanathan U, et al. Med Hypothesis Discov Innov Ophthalmol. 2019; 8(3):163-176). Corticosteroids are often used to control the ophthalmic inflammatory response, however, this treatment is immunosuppressive and can result in uncontrolled pathogen replication, loss of an intact corneal epithelial barrier, increased ocular pressure and eventual deterioration of vision (Fung A T, et al. Clin Exp Ophthalmol. 2020; 48(3):366-401). By contrast, modulation with 5-HT receptor agonists has been shown to have anti-inflammatory and anti-vascularization properties, and the ability to decrease ophthalmic pressure (Foster T, et al. Invest Ophthalmol Vis Sci. 2020; 61(7):429). In some embodiments, a disclosed compound can be used to reduce, or ameliorate, or prevent an ophthalmic disease or disorder, non-limiting examples of which are described herein.

In some embodiments, the ophthalmic disease or disorder is an inflammatory disorder. In some embodiments, the ophthalmic disease or disorder is macular degeneration (e.g., age-related macular degeneration), keratoconjunctivitis, conjunctivitis, keratitis, diabetic retinopathy, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, retinochoroidal neovascularization, or a combination thereof.

In some embodiments, the ophthalmic disease is macular degeneration. In some embodiments, the ophthalmic disease is keratoconjunctivitis. In some embodiments, the ophthalmic disease is conjunctivitis. In some embodiments, the ophthalmic disease is keratitis. In some embodiments, the ophthalmic disease is diabetic retinopathy. In some embodiments, the ophthalmic disease is retinopathy of prematurity. In some embodiments, the ophthalmic disease is polypoidal choroidal vasculopathy. In some embodiments, the ophthalmic disease is ischemic proliferative retinopathy. In some embodiments, the ophthalmic disease is retinitis pigmentosa. In some embodiments, the ophthalmic disease is cone dystrophy. In some embodiments, the ophthalmic disease is proliferative vitreoretinopathy. In some embodiments, the ophthalmic disease is retinal artery occlusion. In some embodiments, the ophthalmic disease is retinal vein occlusion. In some embodiments, the ophthalmic disease is Leber's disease. In some embodiments, the ophthalmic disease is retinal detachment. In some embodiments, the ophthalmic disease is retinal pigment epithelial detachment. In some embodiments, the ophthalmic disease is rubeosis iridis. In some embodiments, the ophthalmic disease is corneal neovascularization. In some embodiments, the ophthalmic disease is retinal neovascularization. In some embodiments, the ophthalmic disease is choroidal neovascularization. In some embodiments, the ophthalmic disease is retinochoroidal neovascularization.

A reduction in inflammation, such as chronic systemic inflammation, may be measured according to various methods available to one of skill. Inflammatory biomarkers may be detected from biological specimens, for example, a subject's blood, such as plasma or serum, or saliva. In one example, inflammation may be detected by measuring high-sensitivity C-reactive protein (CRP) and white blood cell count from a blood test. CRP may also be detected in a saliva sample. Salivary CRP is not synthesized locally in the mouth and may reflect more systemic levels of inflammation compared to other inflammatory biomarkers, such as cytokines (Szabo & Slavish, Psychoneuroendocrin. 202; 124:105069). Additionally clinical pathology data, e.g., hematology data on erythrocyte parameters, platelet count, total number of leukocytes, and leukocyte differentials and morphology, coagulation data on clotting times and fibrinogen, and clinical chemistry data on total protein, albumin and globulin, liver enzymes, renal parameters, electrolytes, and bilirubin can provide an initial indication of the presence and potentially the location of inflammation, in the absence of specific data on immune tissues. See e.g., Germolec et al. Methods Mol Biol. 2018; 1803:57-79 and Luo et al. Clin Lab. 2019 1; 65(3).

### I. EXAMPLES

The following examples are included for illustrative purposes only and are not intended to limit the scope of the invention.

### Example 12: Synthesis of 3-iodo-2,5-dimethoxybicyclo[4.2.0]octa-1,3,5-trien-7-yl) methanamine (Compound 2)

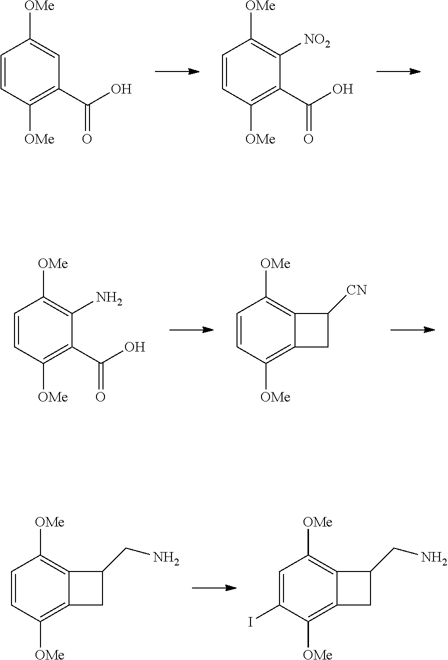 [SMILES: COc1ccc(OC)c(C(=O)O)c1 ; COc1ccc(OC)c([N+](=O)[O-])c1C(=O)O ; COc1ccc(OC)c(C(=O)O)c1N ; COc1ccc(OC)c2c1CC2C#N ; COc1ccc(OC)c2c1CC2CN ; COc1cc(I)c(OC)c2c1C(CN)C2]

### Step 1: 3,6-dimethoxy-2-nitrobenzoic acid

A solution of 2,5-dimethoxybenzoic acid (50.0 g, 274 mmol.) in HNO₃ (1.40 kg, 22.0 mol, 982 mL) was stirred at 0° C. for 0.5 hr. The reaction mixture was quenched by H₂O (300 mL) at 0° C. and filtered. The filter cake was collected to yield 3,6-dimethoxy-2-nitrobenzoic acid (147 g, crude) as a yellow solid. ¹H NMR (400 MHz, DMSO-d₆) δ 7.37 (q, J=9.4 Hz, 2H), 3.85 (s, 3H), 3.81 (s, 3H). LCMS (ESI⁺): m/z 227.9 [M+H]⁺

### Step 2: 2-amino-3,6-dimethoxybenzoic acid

To a solution of 3,6-dimethoxy-2-nitrobenzoic acid (45.0 g, 198 mmol) in EtOH (1 L) was added Pd/C (1.0 g, 10.0% purity) and ammonium formate (50.0 g, 792 mmol) at 0° C. The mixture was stirred at 50° C. for 7 hrs. The reaction mixture was filtered and concentrated. The crude product was triturated with H₂O (1 L) at 25° C. for 2 hrs to yield 2-amino-3,6-dimethoxy-benzoic acid (70.0 g, 355 mmol, 89.6% yield) as a brown solid. ¹H NMR (400 MHz, DMSO-d₆) δ 6.83 (d, J=8.8 Hz, 1H), 6.15 (d, J=8.8 Hz, 1H), 3.75-3.70 (m, 6H) LCMS (ESI⁺): m/z 198.0 [M+H]⁺

### Step 3: 2,5-dimethoxybicyclo[4.2.0]octa-1,3,5-triene-7-carbonitrile

To a solution of 2-amino-3,6-dimethoxy-benzoic acid (50.0 g, 254 mmol) in EtOH (500 mL) was added HCl (12 mL) and isopentyl nitrite (59.0 g, 507 mmol, 68 mL) at 0° C. for 0.2 hr, followed by MTBE (250 mL). The filtrate was concentrated, followed by DCE (450 mL), 2-methyloxirane (30 mL) and prop-2-enenitrile (45.0 g, 840 mmol, 56 mL). The mixture was stirred at 80° C. for 2 hrs. The reaction mixture was concentrated, quenched by sat. aq. NaCl (1 L) and extracted with EtOAc (3×1 L). The combined organic layers were dried over Na₂SO₄, filtered, concentrated and purified by column chromatography (SiO₂, Petroleum ether:EtOAc=95:5 to 93:7) to yield 2,5-dimethoxybicyclo[4.2.0]octa-1,3,5-triene-7-carbonitrile (1.40 g, 7.40 mmol, 2.9% yield) as a white solid. ¹H NMR (400 MHz, Chloroform-d) δ 6.66 (q, J=9.1 Hz, 2H), 4.20 (dd, J=2.6, 5.5 Hz, 1H), 3.81 (s, 3H), 3.74 (s, 3H), 3.69-3.63 (m, 1H), 3.55-3.50 (m, 1H) LCMS (ESI⁺): m/z 188.1 [M−H]⁺

### Step 4: (2,5-dimethoxybicyclo[4.2.0]octa-1,3,5-trien-7-yl)methanamine

To a solution of 2,5-dimethoxybicyclo[4.2.0]octa-1,3,5-triene-7-carbonitrile (200 mg, 1.10 mmol) in THF (5 mL) was added BH₃·THF (1 M, 2.1 mL) at 0° C. The mixture was stirred at 50° C. for 6 hrs under N₂ atmosphere, quenched by MeOH (10 mL) at 0° C. and concentrated to yield (2,5-dimethoxy-7-bicyclo[4.2.0]octa-1,3,5-trienyl)methanamine (200 mg, crude) as a yellow oil. LCMS (ESI): m/z 194.1 [M+H]⁺

### Step 5: (3-iodo-2,5-dimethoxybicyclo[4.2.0]octa-1,3,5-trien-7-yl)methanamine (2)

To a solution of (2,5-dimethoxy-7-bicyclo[4.2.0]octa-1,3,5-trienyl)methanamine (170 mg, 880 μmol) in EtOH (2 mL) was added AgOTf (249 mg, 968 μmol) for 0.3 hr, followed by I₂ (246 mg, 968 μmol, 195 μL). The mixture was stirred at 25° C. for 2 hrs, concentrated and purified by prep-HPLC (column: Phenomenex luna C18 100*40 mm*5 μm; mobile phase: [H₂O (0.04% HCl)—ACN]; gradient: 10-30% B over 8.0 mins) to yield (3-iodo-2,5-dimethoxy-7-bicyclo[4.2.0]octa-1,3,5-trienyl)methanamine (3) (130 mg, 402 μmol, 45.7% yield, 98.7% purity) as a white solid.

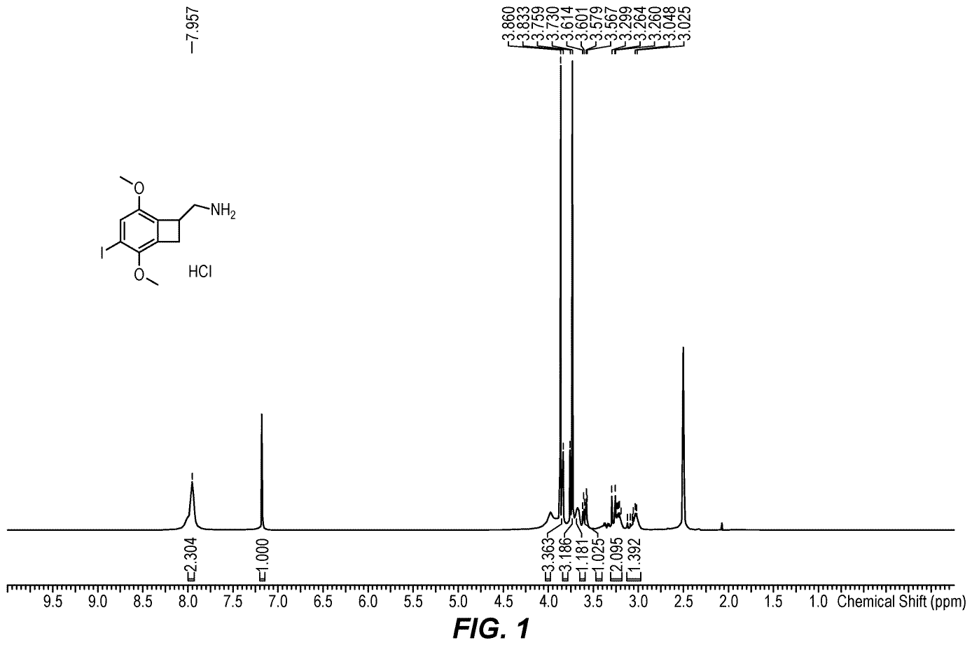

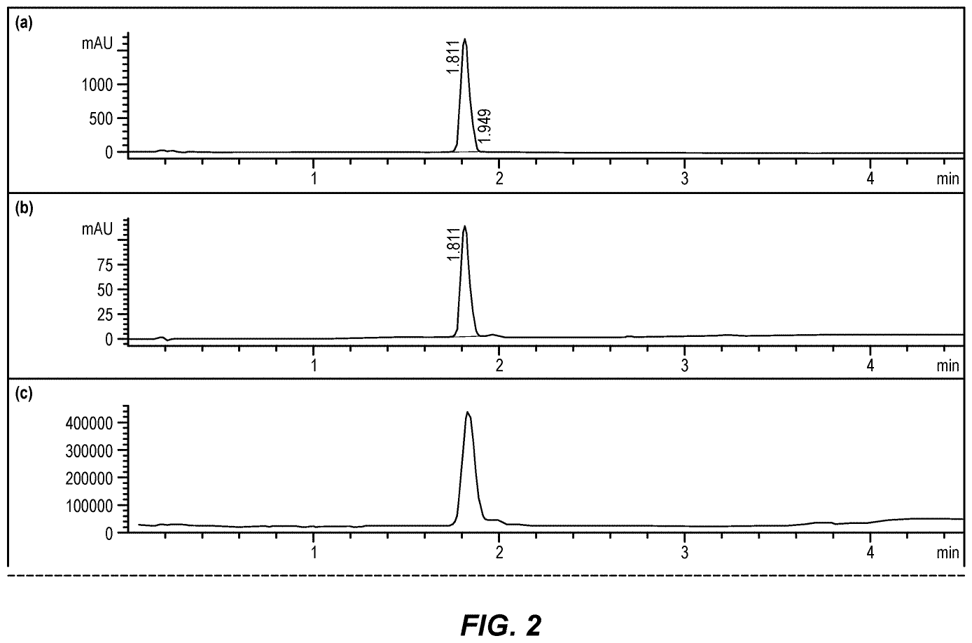

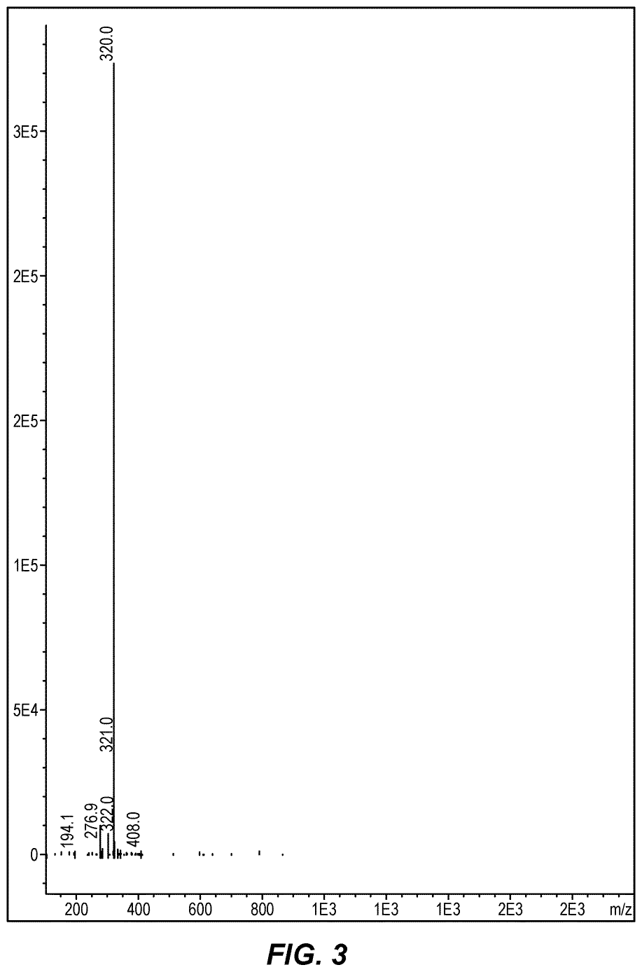

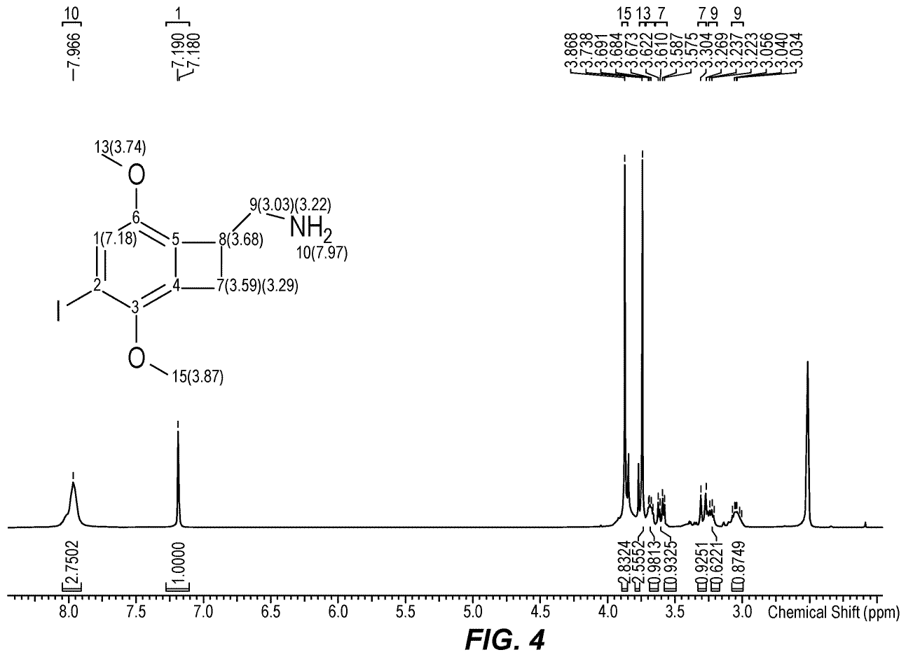

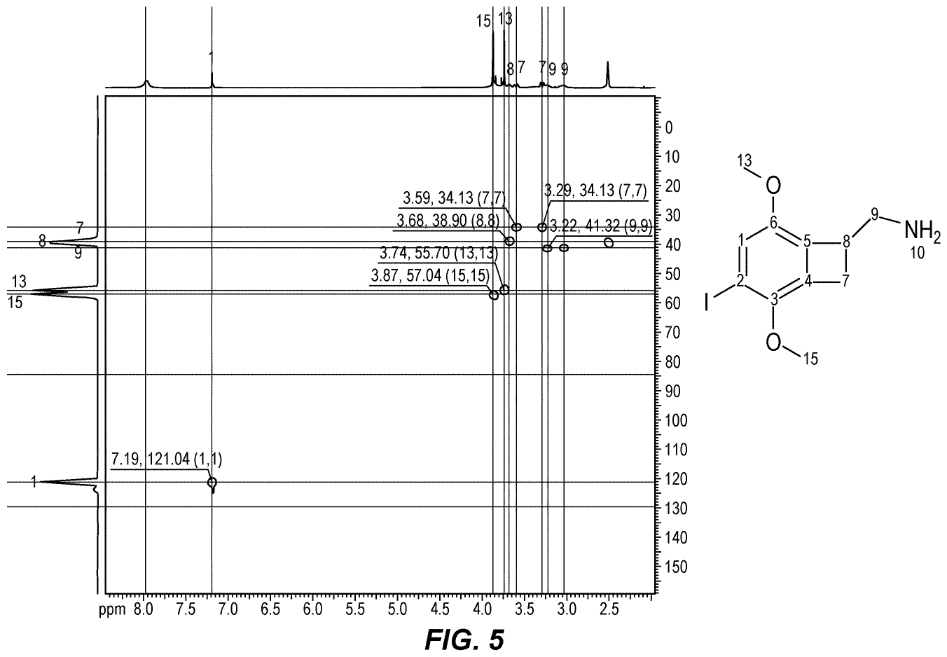

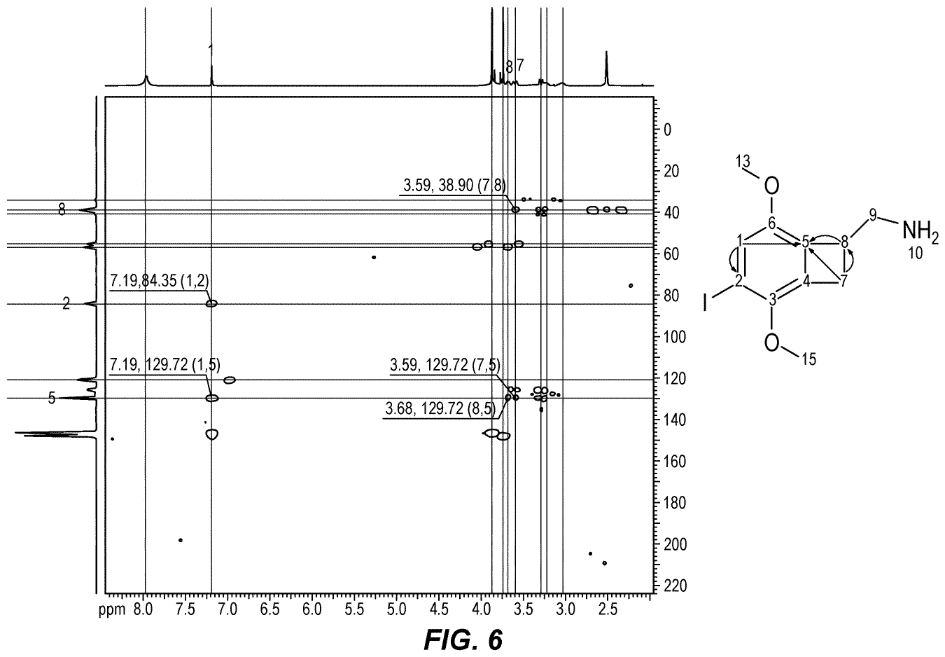

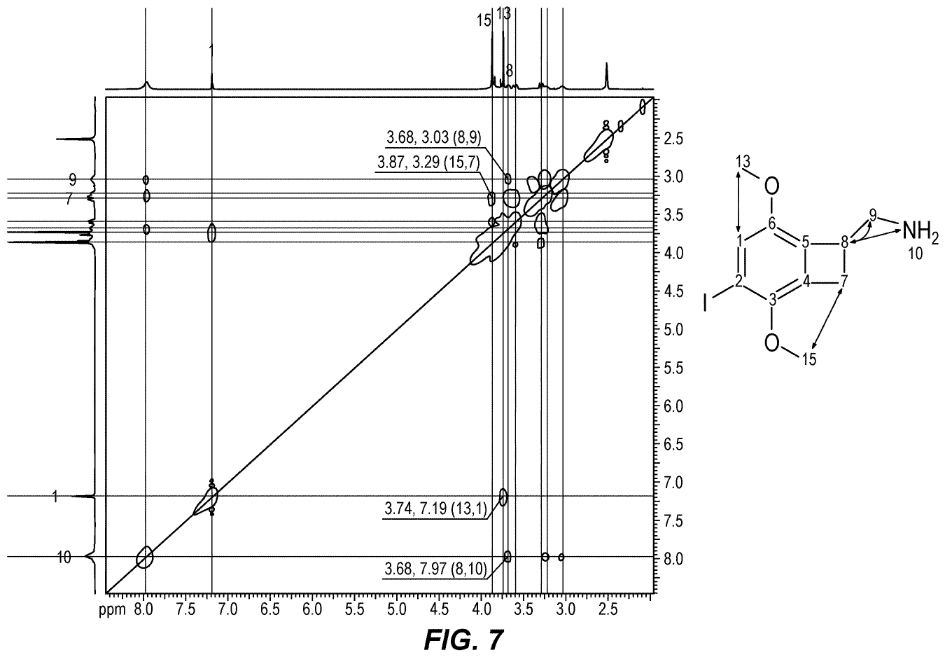

Compound 2 was characterized by 1-dimensional 1H nuclear magnetic resonance (NMR); see FIG. 1 and FIG. 4. ¹H NMR (400 MHz, DMSO-d₆) δ 7.96 (br s, 2H), 7.21-7.15 (m, 1H), 3.88-3.82 (m, 3H), 3.76-3.71 (m, 3H), 3.71-3.65 (m, 1H), 3.59 (dd, J=5.0, 13.9 Hz, 1H), 3.32-3.19 (m, 2H), 3.13-2.98 (m, 1H). Compound 2 was also characterized by liquid-chromatography mass-spectrometry (LCMS); see FIG. 2 and FIG. 3. LCMS (ESI⁺): m/z 320.0 [M+H]⁺. The regiochemistry of Compound 2 was also confirmed by 2-dimensional NMR experiments; see FIG. 5 for an exemplary Heteronuclear Single Quantum Correlation-Distortionless Enhancement by Polarization Transfer (HSQC-DEPT) NMR spectrum in DMSO-d₆, FIG. 6 for an exemplary Heteronuclear Multiple Bond Correlation (HMBC) NMR spectrum in DMSO-d₆, and FIG. 7 for an exemplary Nuclear Overhauser Effect Spectroscopy (NOESY) NMR spectrum in DMSO-d₆.

### Example 13: Synthesis of (3-ethyl-2,5-dimethoxybicyclo[4.2.0]octa-1,3,5-trien-7-yl) methanamine (Compound 4)

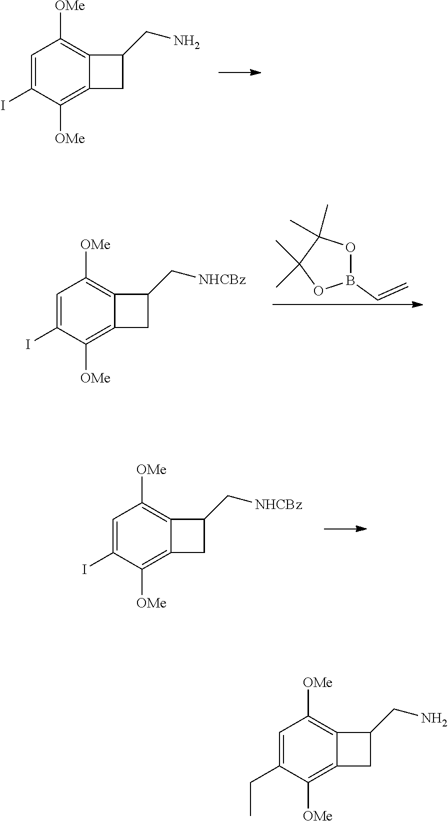

### Step 1: benzyl ((3-iodo-2,5-dimethoxybicyclo[4.2.0]octa-1,3,5-trien-7-yl)methyl) carbamate

To a solution of (3-iodo-2,5-dimethoxy-7-bicyclo[4.2.0]octa-1,3,5-trienyl)methanamine (2.50 g, 7.80 mmol) in DCM (25 mL) was added TEA (2.40 g, 24.0 mmol, 3 mL) and CbzCl (2.00 g, 12.0 mmol, 2 mL). The mixture was stirred at 0° C. for 1 hr, quenched by H₂O (50 mL) and extracted with DCM (3×100 mL). The combined organic layers were dried over Na₂SO₄, filtered, concentrated and purified by column chromatography (SiO₂, Petroleum ether:EtOAc=1:0 to 93:7) to yield benzyl N-[(3-iodo-2,5-dimethoxy-7-bicyclo[4.2.0]octa-1,3,5-trienyl)methyl] carbamate (915 mg, 2.00 mmol, 25.7% yield) as a yellow oil. ¹H NMR (400 MHz, DMSO-d₆) δ 7.41-7.30 (m, 5H), 7.11 (s, 1H), 5.06-4.97 (m, 2H), 3.87-3.79 (m, 3H), 3.75-3.67 (m, 3H), 3.59-3.43 (m, 3H), 3.36-3.24 (m, 2H) LCMS (ESI⁺): m/z 454.1 [M+H]⁺

### Step 2: benzyl ((2,5-dimethoxy-3-vinylbicyclo[4.2.0]octa-1,3,5-trien-7-yl)methyl) carbamate

To a solution of benzyl N-[(3-iodo-2,5-dimethoxy-7-bicyclo[4.2.0]octa-1,3,5-trienyl)methyl] carbamate (300 mg, 662 μmol) and 4,4,5,5-tetramethyl-2-vinyl-1,3,2-dioxaborolane (122 mg, 794 μmol, 135 μL) in THF (3 mL), H₂O (1 mL) was added Pd(dppf)Cl₂ (48.0 mg, 66.0 μmol) and K₃PO₄ (421 mg, 2.00 mmol.). The mixture was stirred at 80° C. for 1 hr under N₂ atmosphere, quenched by H₂O (50 mL) and extracted with EtOAc (3×50 mL). The combined organic layers were dried over Na₂SO₄, filtered, concentrated and purified by column chromatography (SiO₂, Petroleum ether:EtOAc=1:9 to 9:1) to yield benzyl N-[(2,5-dimethoxy-3-vinyl-7-bicyclo[4.2.0]octa-1,3,5-trienyl)methyl]carbamate (180 mg, 509 μmol, 51.4% yield) as a white solid. LCMS (ESI⁺): m/z 354.0 [M+H]⁺

### Step 3: (3-ethyl-2,5-dimethoxybicyclo[4.2.0]octa-1,3,5-trien-7-yl)methanamine (4)

To a solution of benzyl N-[(2,5-dimethoxy-3-vinyl-7-bicyclo[4.2.0]octa-1,3,5-trienyl)methyl] carbamate (170 mg, 483 μmol) in EtOAc (5 mL) was added 10.0% Pd/C (10.0 mg). The mixture was stirred at 25° C. for 2 hrs under H₂ atmosphere, filtered, concentrated and purified by prep-HPLC (Phenomenex luna C18 100*40 mm*5 μm; mobile phase: [H₂O (0.04% HCl)-ACN]; gradient: 5%-30% B over 8.0 mins) to yield (3-ethyl-2,5-dimethoxy-7-bicyclo[4.2.0]octa-1,3,5-trienyl)methanamine (63.0 mg, 281 μmol, 58.5% yield, 99.1% purity) as a yellow solid.

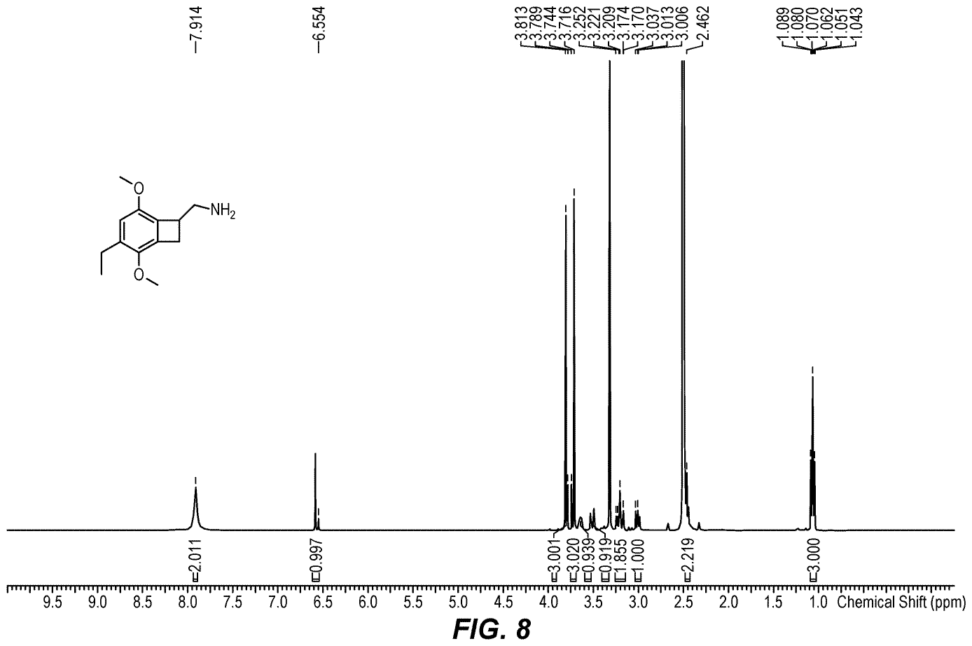

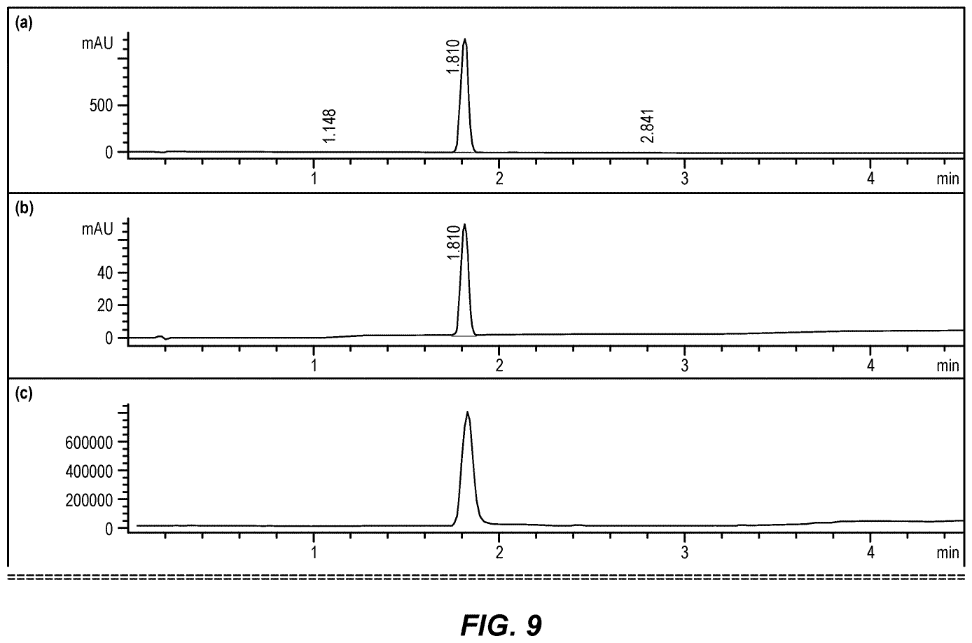

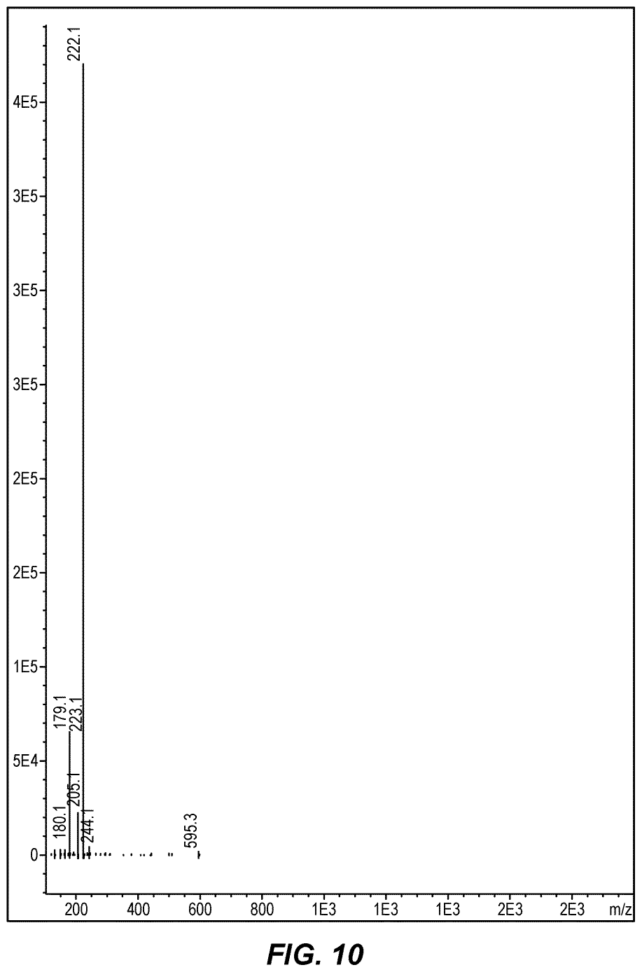

Compound 4 was characterized by ¹H NMR; see FIG. 8 for an exemplary spectrum in DMSO-d₆. ¹H NMR (400 MHz, DMSO-d₆) δ 7.91 (br s, 2H), 6.55 (s, 1H), 3.82-3.78 (m, 3H), 3.75-3.70 (m, 3H), 3.68-3.62 (m, 1H), 3.55-3.48 (m, 1H), 3.26-3.15 (m, 2H), 3.01 (dd, J=9.7, 12.5 Hz, 1H), 2.46 (s, 2H), 1.10-1.03 (m, 3H). Compound 4 was also characterized by LCMS; see FIG. 9 and FIG. 10. LCMS (ESI⁺): m/z 222.1 [M+H]⁺

### Example 14: Synthesis of (3-isobutyl-2,5-dimethoxybicyclo[4.2.0]octa-1,3,5-trien-7-yl)methanamine (Compound 8)

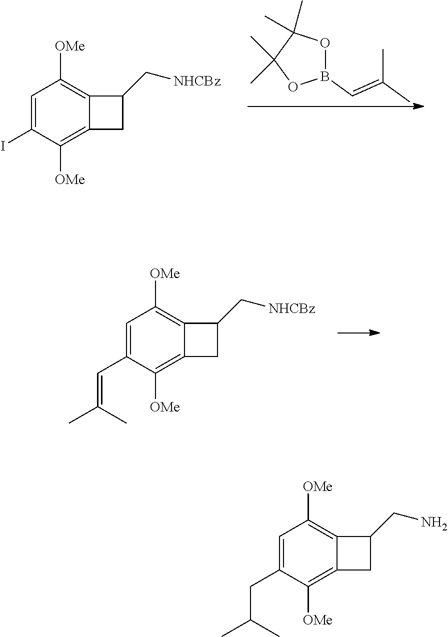

### Step 1: benzyl ((2,5-dimethoxy-3-(2-methylprop-1-en-1-yl) bicyclo[4.2.0]octa-1,3,5-trien-7-yl)methyl) carbamate

A mixture of benzyl N-[(3-iodo-2,5-dimethoxy-7-bicyclo[4.2.0]octa-1,3,5-trienyl)methyl] carbamate (300 mg, 662 μmol), 4,4,5,5-tetramethyl-2-(2-methylprop-1-enyl)-1,3,2-dioxaborolane (145 mg, 794 μmol), Pd(dppf)Cl₂ (48.0 mg, 66.0 μmol) and K₃PO₄ (421 mg, 2.00 mmol) in THF (3 mL), H₂O (1 mL) was stirred at 80° C. for 1 hr under N₂ atmosphere. The residue was quenched by H₂O (50 mL) and extracted with EtOAc (3×50 mL). The combined organic layers were dried over Na₂SO₄, filtered, concentrated and purified by column chromatography (SiO₂, Petroleum ether:EtOAc=95:5 to 9:1) to yield benzyl N-[[2,5-dimethoxy-3-(2-methylprop-1-enyl)-7-bicyclo [4.2.0]octa-1,3,5-trienyl]methyl]carbamate (220 mg, 577 μmol, 87.1% yield) as a yellow oil. 1H NMR (400 MHz, DMSO-d₆) δ 7.39-7.27 (m, 5H), 6.50-6.42 (m, 1H), 6.15-6.08 (m, 1H), 5.09-4.96 (m, 2H), 3.79-3.74 (m, 3H), 3.73-3.68 (m, 3H), 3.58-3.39 (m, 3H), 3.22-3.00 (m, 2H), 1.82 (s, 3H), 1.71 (s, 3H) LCMS (ESI⁺): m/z 381.9 [M+H]⁺

### Step 2: (3-isobutyl-2,5-dimethoxybicyclo[4.2.0]octa-1,3,5-trien-7-yl)methanamine (8)

To a solution of benzyl N-[[2,5-dimethoxy-3-(2-methylprop-1-enyl)-7-bicyclo[4.2.0]octa-1,3,5-trienyl]methyl]carbamate (150 mg, 393 μmol) in EtOAc (5 mL) was added Pd/C (10.0 mg, 10.0%). The mixture was stirred at 25° C. for 2 hrs under H₂ atmosphere, filtered, concentrated and purified by prep-HPLC (column: Phenomenex luna C18 100*40 mm*5 μm; mobile phase: [H₂O (0.04% HCl)-ACN]; gradient: 5%-30% B over 8.0 mins) to yield (3-isobutyl-2,5-dimethoxy-7-bicyclo[4.2.0]octa-1,3,5-trienyl)methanamine (53.0 mg, 210 μmol, 53.4% yield, 99.5% purity) as a yellow solid.

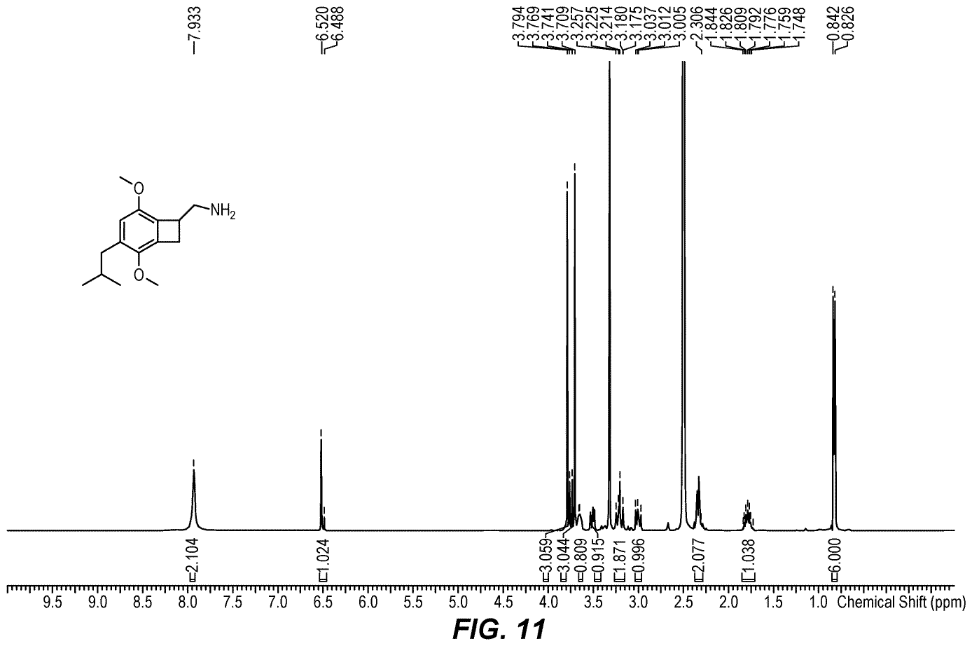

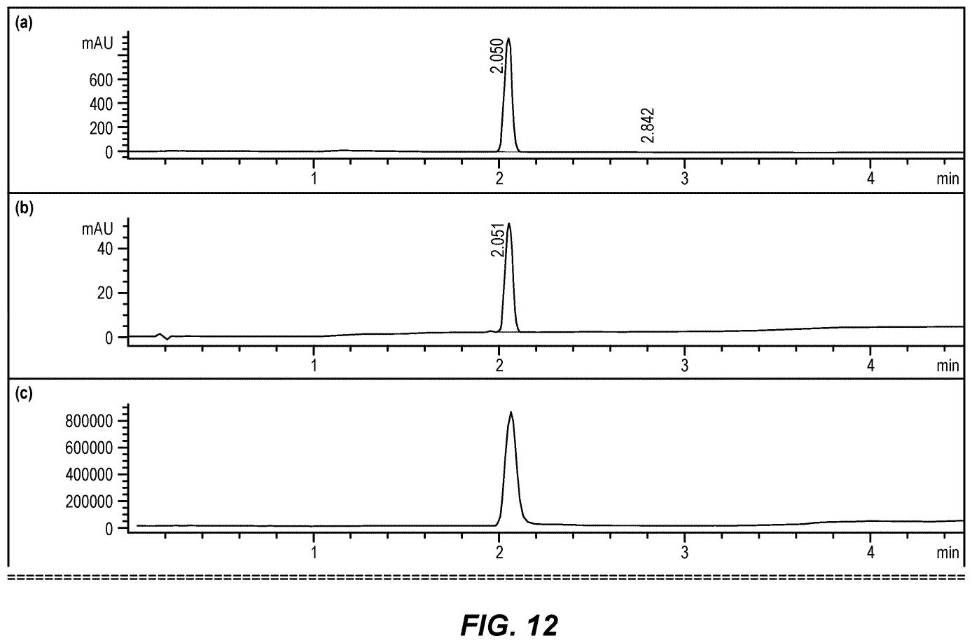

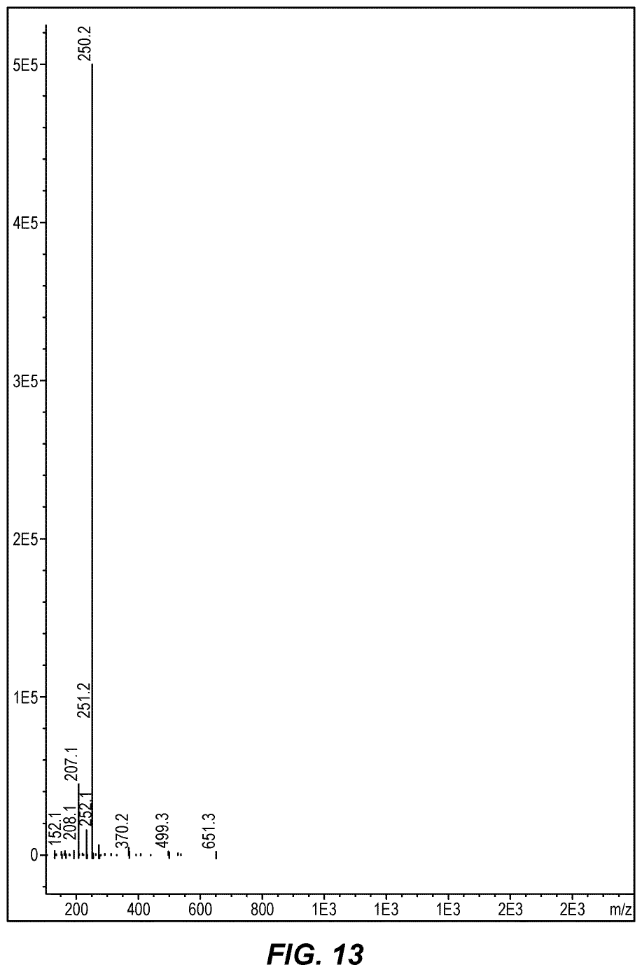

Compound 8 was characterized by ¹H NMR; see FIG. 11 for an exemplary spectrum in DMSO-d₆. ¹H NMR (400 MHz, DMSO-d₆) δ 7.93 (br s, 2H), 6.55-6.47 (m, 1H), 3.81-3.76 (m, 3H), 3.75-3.69 (m, 3H), 3.66 (br dd, J=2.9, 5.0 Hz, 1H), 3.56-3.48 (m, 1H), 3.27-3.16 (m, 2H), 3.01 (dd, J=9.7, 12.5 Hz, 1H), 2.31 (br s, 2H), 1.85-1.71 (m, 1H), 0.83 (d, J=6.6 Hz, 6H). Compound 8 was also characterized by LCMS; see FIG. 11 and FIG. 12. LCMS (ESI⁺): m/z 250.2 [M+H]⁺

### Example 15: In Vitro Receptor Binding and Functional Activity of Compounds

5-HT_{2A}, 5-HT_{2B}, and 5-HT_{2C} In Vitro Receptor Binding Assays:

Membrane was extracted from 5-HT2A/HEK293, 5-HT2B/HEK293 and 5-HT2C/HEK293 cells. The reference compounds and screening compounds were 4-fold serially diluted in 100% DMSO for 8 points. Transfer 1 μL of serial diluted references and screening compounds to the assay plates. Then added 100 μL/well of membrane and 100 μL/well of radioligand 3H-LSD. Incubated at room temperature for 1 hour. Filtered the reaction mixture through the GF/C plate using PerkinElmer Filtermate Harvester and wash the plates. Dried the filter plate for 1 hour at 50°C. Sealed the bottom of the filter plate using Perkin Elmer Unifilter-96 backing seal tape. Added 50 μL of Perkin Elmer Microscint 20 cocktail to each well of assay plate and counted 3H trapped on filter plate using Perkin Elmer MicroBeta2 Reader.

### 5-HT_{2B}IP-One Assay:

Intracellular accumulation of IP1 was measured using an IP-One HTRF) assay kit (Cat. #62IPAPEJ, Cisbio) at WuXi AppTec Co. Ltd. (Hong Kong) Discovery Biology Unit according to their standard protocols. Briefly, 5-HT2B/HEK293 were plated in a 384-well plate and incubated at 37° C. and 5% CO₂ overnight. The reference compounds and screening compounds were 3.16-fold serially diluted in 100% DMSO for 10 points using Bravo. 70 nL of compounds were added to the cell plate using Echo555. Incubated for 60 minutes at 37° C. Added 3 μL of IP1 d2 Reagent working solution and 3 μL of IP1 Tb Cryptate Antibody working solution to all wells. The plates were incubated for 1 hour at room temperature and read on for fluorescence at 620 nm and 665 nm on an EnVision Multimode Plate Reader (PerkinElmer). The ratio of the acceptor and donor emission signals (665/620) were calculated for each individual well and substituted into the standard curve to obtain the log concentration of IP level. After converting to the antilog base IP-1, the average background control signal was subtracted from each well and values were normalized to the maximal response of 5-HT at 3 μM (100%). % MAX was calculated by taking the average normalized maximal response at the highest concentration tested. The data were then analyzed using the four-parameter nonlinear regression curve-fitting function in GraphPad Prism 5 (GraphPad Software, San Diego, CA) to generate potency (EC₅₀) values. Parameter constraint “Top=100” was used in the analysis of 5-HT2B IP1. The represented plots show normalized IP-1 values versus compound concentrations; the corresponding numerical data are parameter estimates for the concentration-response curve using the four-parameter nonlinear regression curve-fitting function in GraphPad Prism 10. “Top=100” parameter constraint was not enabled for the represented plot analysis.”

5-HT_{2A} and 5-HT_{2C} IP-One Assay:

Intracellular accumulation of IP1 was measured using an IP-One HTRF assay kit (Cat. #62IPAPEJ, Cisbio) at WuXi AppTec Co. Ltd. (Hong Kong) Discovery Biology Unit according to their standard protocols. Briefly, the reference compounds and screening compounds were 3.16-fold serially diluted in 100% DMSO for 10 points using Bravo. 70 nL of compounds were added to the assay plate using Echo555. Added 14 μL/7500 cells/well of 5-HT_{2A}-expressing HEK293 or 5-HT_{2C}-expressing HEK293 to the assay plate and incubate for 60 min at 37° C. Added 3 μL of IP1 d2 Reagent working solution and 3 μL of IP1 Tb Cryptate Antibody working solution to all wells. The plates were incubated for 1 hour at room temperature and read for fluorescence at 620 nm and 665 nm on an En Vision Multimode Plate Reader (PerkinElmer). The ratio of the acceptor and donor emission signals (665/620) were calculated for each individual well and substituted into the standard curve to obtain the log concentration of IP level. After converting to the antilog base IP-1, the average background control signal was subtracted from each well and values were normalized to the maximal response of 5-HT at 3 μM (100%). % MAX was calculated by taking the average normalized maximal response for each compound at the highest concentration tested. The data were then analyzed using the four-parameter nonlinear regression curve-fitting function in GraphPad Prism 5 (GraphPad Software, San Diego, CA) to generate potency (EC₅₀) values. The represented plots show normalized IP-1 values versus compound concentrations; the corresponding numerical data are parameter estimates for the concentration-response curve using the four-parameter nonlinear regression curve-fitting function in GraphPad Prism 10.

### Results:

Compound 2

| Binding ([³H]-LSD)-Kᵢ (% lnh) | IP-One EC₅₀ (% MAX) |
|---|---|
| 2A: 17 nM (96%) | 2A: 10 nM (127%) |
| 2B: 28 nM (77%) | 2B: >300 nM (49%) |
| 2C: 3 nM (96%) | 2C: 41 nM (122%) |

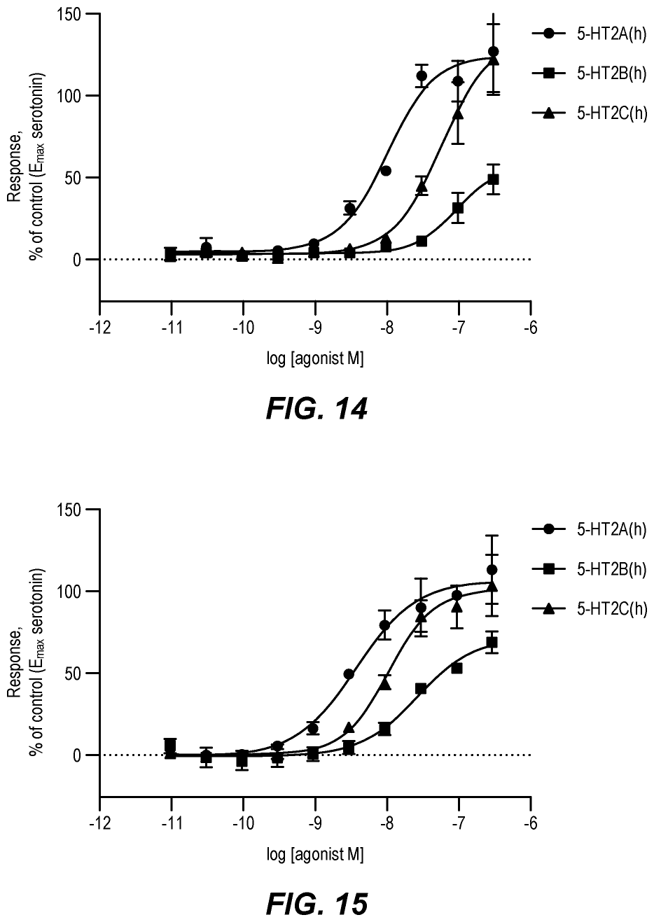

For the IP-One assays, FIG. 14 shows a plot of Response (% of control; Emax serotonin) vs. log [agonist M] for Compound 2, for 5-HT_{2A}, 5-HT_{2B}, and 5-HT_{2C}. Corresponding numerical data for this assay were:

| | 5-HT2A | 5-HT2B | 5-HT2C |
|---|---|---|---|
| | (h) | (h) | (h) |
| Bottom | 5.304 | 3.780 | 3.427 |
| Top | 124.3 | 56.20 | 136.3 |
| LogEC50 | −7.996 | −7.050 | −7.237 |
| HillSlope | 1.338 | 1.531 | 1.279 |
| EC50 | 1.008e−008 | 8.906e−008 | 5.799e−008 |

Compound 2 has low nanomolar affinity at 5-HT_{2A}, 5-HT_{2B}, and 5-HT_{2C}, approximately 1.6-fold affinity selectivity for 5-HT_{2A} over 5-HT_{2B}, and approximately 6-fold affinity selectivity for 5-HT_{2C} over 5-HT_{2A}. Compound 2 has minimal functional activity at 5-HT_{2B} and is approximately 4-fold functionally selective for 5-HT_{2A} over 5-HT_{2C}.

Compound 4

| Binding ([³H]-LSD)-Kᵢ (% lnh) | IP-One EC₅₀ (% MAX) |
|---|---|
| 2A: 26 nM (90%) | 2A: 4 nM (114%) |
| 2B: 43 nM (73%) | 2B: 70 nM (70%) |
| 2C: 4 nM (89%) | 2C: 11 nM (104%) |

For the IP-One assays, FIG. 15 shows a plot of Response (% of control; Emax serotonin) vs. log [agonist M] for Compound 4, for 5-HT_{2A}, 5-HT_{2B}, and 5-HT_{2C}. Corresponding numerical data for this assay were:

| | 5-HT2A | 5-HT2B | 5-HT2C |
|---|---|---|---|
| | (h) | (h) | (h) |
| Bottom | −0.4425 | −0.2984 | 1.236 |
| Top | 107.6 | 72.83 | 101.7 |
| LogEC50 | −8.424 | −7.563 | −7.967 |
| HillSlope | 1.033 | 1.088 | 1.410 |
| EC50 | 3.765e−009 | 2.735e−008 | 1.079e−008 |

Compound 4 has low nanomolar affinity at 5-HT_{2A}, 5-HT_{2B}, and 5-HT_{2C}, approximately 1.6-fold affinity selectivity for 5-HT_{2A} over 5-HT_{2B}, and approximately 6.5-fold affinity selectivity for 5-HT_{2C} over 5-HT_{2A}. Compound 4 has significant functional activity at 5-HT_{2B}, approximately 2.75-fold functional selectivity for 5-HT_{2A} over 5-HT_{2C}, and approximately 17.5-fold functional selectivity for 5-HT_{2A} over 5-HT_{2B}.

Compound 8

| Binding ([³H]-LSD)-Kᵢ (% lnh) | IP-One EC₅₀ (% MAX) |
|---|---|
| 2A: 133 nM (65%) | 2A: 2 nM (113%) |
| 2B: >95 nM (29%) | 2B: 297 nM (51%) |
| 2C: 4 nM (93%) | 2C: 9 nM (117%) |

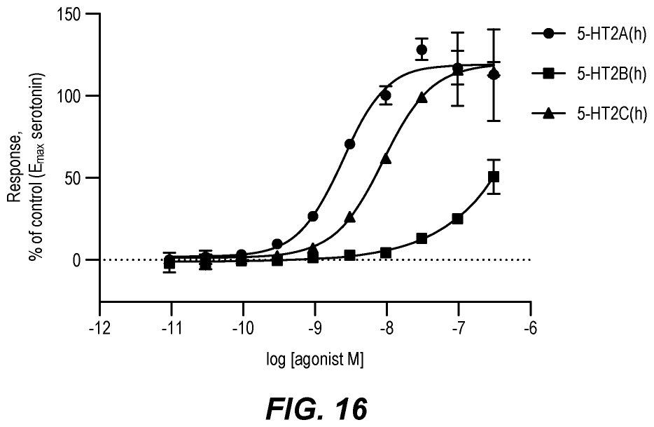

For the IP-One assays, FIG. 16 shows a plot of Response (% of control; Emax serotonin) vs. log [agonist M] for Compound 8, for 5-HT_{2A}, 5-HT_{2B}, and 5-HT_{2C}. Corresponding numerical data for this assay were:

| | 5-HT2A | 5-HT2B | 5-HT2C |
|---|---|---|---|
| | (h) | (h) | (h) |
| Bottom | 2.988 | −0.5387 | 2.175 |
| Top | 119.7 | 51.17 | 119.8 |
| LogEC50 | −8.619 | −3.583 | −8.059 |
| HillSlope | 1.426 | 0.5893 | 1.285 |
| EC50 | 2.403e−009 | 0.0002613 | 8.731e−009 |

Compound 8 has low nanomolar affinity at 5-HT_{2C} and approximately 33-fold affinity selectivity for 5-HT_{2C} over 5-HT_{2A}. Compound 8 has moderate functional activity at 5-HT_{2B}, has approximately 4.5-fold functional selectivity for 5-HT_{2A} over 5-HT_{2C}, and approximately 148.5-fold functional selectivity for 5-HT_{2A} over 5-HT_{2B}.

Future work will include testing comparator compounds (e.g., 3-bromo-3,6-dimethoxybenzocyclobuten-1-yl)methylamine, 4-bromo-3,6-dimethoxybenzocyclobuten-1-yl)methylamine), which may exhibit lower potency at 5-HT_{2A}, or reduced affinity or functional selectivity for 5-HT_{2A} or 5-HT_{2C} over 5-HT_{2B}, as compared to a disclosed compound.

### Example 16: Anti-Inflammatory Properties of Compounds

The anti-inflammatory properties of disclosed compounds are assessed in a mouse model of allergic asthma according to methods described in Flanagan et al. ACS Pharmacology & Translational Science 2020, 4(2), 488-502.

Procedure: The respiratory pathogen-free Brown Norway (RijHsd-BN) rats used in this Example are housed singly in a pathogen-free animal facility with free access to food and water on a 12 h/12 h light/dark cycle. Animal protocols are prepared in accordance with the Guide for the Care and Use of Laboratory Animals (Committee for the Update of the Guide for the Care and Use of Laboratory Animals, National Academies Press, Washington, DC (2011)). The rats are allowed to acclimate at least 1 week prior to initiation of sensitization with chicken ovalbumin grade V (OVA).

For sensitization, Brown Norway rats (7-9 weeks old) are i.p. injected with (500 μL) of 2.0 mg of chicken OVA emulsified in 2.0 mL of Imject Alum [Al(OH)₃/Mg(OH)₂] on days 0 and 7, as described in Elwood et al. J Allergy Clin Immunol. 1991, 88(6), 951-60. OVA exposure methods are based on a previously described mouse model of acute asthma (Nau et al. Am J Physiol. Lung Cellular Mo Physiol. 2015, 308(2), L191-198).

OVA-alone treated rats are exposed to 3 times weekly exposure of 10.0 mg of OVA slowly dissolved in 10.0 mL of 0.9% sterile saline solution in a 15 L (38.00× 19.05× 19.7 cm) acrylic induction chamber. No more than 6 animals are exposed in the chamber per challenge. OVA aerosol was generated using an ultrasonic nebulizer in conjunction with a Pari Proneb pump at a 1.0% OVA concentration for a total duration of 30 min, as described in Palmans et al. Am. J. Respir Crit Care Med. 2000, 161, 627-635.

For drug exposures, rats are exposed in groups of 3-4 rats/group to the appropriate concentration of drug dissolved in a total volume of 4.5 mL of sterile saline using an inExpose nose-only inhalation system 30 min prior to each OVA challenge. Each 4.5 mL of sample is aerosolized using a nebulizer in conjunction with a Pari Proneb pump. Exposures last 15 min. All respiratory parameters are measured 48 h after the final OVA exposure.

To minimize the impact of circadian influences, all respiratory recordings are performed between 10 am and 3 μm (Lai et al. Bio-protocol. 2017, 7(12), e2343; Lai et al. J Neuro Sci. 2016, 36(50), 12661-12676; Pazhoohan et al. PLOS One. 2017, 12(10), e0187249). For measurement of airway responsiveness to MeCh, a noninvasive bias flow ventilated whole body plethysmography system is used in spontaneously breathing, unrestrained rodents. The plethysmograph is ventilated by a continuous flow of 2.5 L/min. A differential pressure transducer is connected on one pole to the main chamber and on the second pole to a reference chamber. The transducer measures pressure differences between both chambers as caused by the respiratory cycle, mainly inhalation and exhalation. Computer software provides a breath-by-breath analysis of pressure signals and transforms pressure differences via computerized calculations to a dimensionless empirically established value, enhanced pause or PenH.

Numerous experiments (Nau et al. Am J Physiol. Lung Cellular Mol Physiol 2015, 308(2), L191-198; Flanagan et al., Life Sci. 2019, 236, 116790; Hamelmann et al., Am. J. Respir. Crit. Care Med. 1997, 156, 766-775; Djuric et al., Brain, Behav. Immun. 1998, 12(4), 272-84) have shown PenH to be a reliable and sensitive measure of bronchoconstriction and a superior measure in assessing the degree of bronchoconstriction compared to other derived parameters such as box pressure or box flow (Djuric et al., Brain, Behav. Immun. 1998, 12(4), 272-84), and it faithfully reproduces the results of forced respiratory techniques such as flexiVent (flexiVent, SCIREQ, Montreal, CA) (Nau et al. American Journal of Physiology. Lung Cellular and Molecular Physiology 2015, 308(2), L191-198).

For the assay, the chamber pressure signal is calibrated by dynamic injection of 5 mL of room air via syringe. Rats are then placed in the chamber, where baseline data is recorded for 5 min following a 10 min habituation period in the plethysmograph. After measurement of baseline PenH, either aerosolized saline (0.9% NaCl Solution) or an aqueous solution of MeCh in increasing concentrations (4, 8, 16, 32 mg/mL) is nebulized through an inlet of the plethysmography chamber for 3 min, followed by measurements of PenH values for 3 min. A vibrating-mesh nebulizer is used to generate aerosol. Following recordings, to prevent a MeCh gradient there is a wash-out period of 7 min in which the rat is provided with fresh air. Data are expressed as the mean SEM of maximal PenH values per group.

Results: Results may show that certain disclosed compounds possess potent anti-inflammatory properties, and more specifically that they may reduce PenH max values and suppress pulmonary inflammation.

### Example 17: Assessment of Ocular Inflammation Following Application of Compounds

Purpose: Ocular inflammation and uveitis encompass potentially sight-threatening diseases with local and systemic etiologies. Cytokines, e.g. IL-6 (Ghasemi, Ocul Immunol Inflamm. 2018; 26(1):37-50) and IL-8 (Ghasemi et al. Ocul Immunol Inflamm. 2011 December; 19(6):401-12), and neuropeptides, e.g., substance P (Bignami et al. Curr Drug Targets. 2016; 17(11):1265-74), can contribute to ocular inflammation.

Methods: Ocular inflammation is assessed according to known methods with modifications. For example, ocular inflammation can be assessed in induced models of uveitis (see, e.g., WO2015074137A1, which describes an endotoxin-induced model in Example 1 and an LPS-induced model in Example 2), a chemical cauterization model of corneal inflammation (see, e.g., Example 4 of WO2015074137A1), or in human subjects at risk of experiencing or currently experiencing such inflammation.

Results & Significance: Application of a disclosed compound, such as topical application, can prevent and/or reduce ocular inflammation. Reductions in ocular inflammation may lead to improvements in symptomatology associated with ocular inflammation, including but not limited to eye redness, pain, and alterations in sight, e.g., blurred vision.

### Example 18: In Vitro Metabolic Stability of Compounds

The purpose of this experiment is to assess the metabolic stability of disclosed compounds in an in vitro assay. The liver is a major site of drug metabolism in the body, and liver microsomes, hepatocytes, and liver S9 fractions can be used to determine the in vitro intrinsic clearance of a compound. See, e.g., Ackley et al., Metabolic Stability Assessed by Liver Microsomes and Hepatocytes. In Yan & Caldwell (eds) Optimization in Drug Discovery. Methods Pharmacol Toxicol. Humana Press, and Richardson et al., Drug Metab Lett. 2016; 10(2):83-90).

Methods: A liver microsomal stability assay is performed according to available methods, e.g., in accordance with the methods described in US 2008/0045588 with modifications. Briefly, the assay is conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds are prepared as solutions in 20% acetonitrile-water and added to the assay mixture (final assay concentration 5 microgram per mL) and incubated at 37° C. Final concentration of acetonitrile in the assay should be <1%. Aliquots (50 μL) are taken out at times 0, 15, 30, 45, and 60 min, and diluted with ice cold acetonitrile (200 μL) to stop the reactions. Samples are centrifuged at 12,000 RPM for 10 min to precipitate proteins. Supernatants are transferred to microcentrifuge tubes and stored for LC/MS/MS analysis of the degradation half-life of the test compounds.

Results & Significance; Results show a measurement of the in vitro intrinsic clearance of disclosed compounds. Such data provides a prediction of the metabolic stability and clearance of the compounds.

### Example 19: In Vivo Assessment of Behavioral Effects of Compounds Using HTR

The mouse head-twitch response (HTR) is a behavioral test that reflects 5-HT_{2A} receptor activation and can be predictive of psychedelic effects in humans (Halberstadt et al. J Psychopharmacol. 2011; 25(11): 1548-1561). HTR is widely used as a behavioral surrogate for human psychedelic effects for its ability to reliably distinguish psychedelic from non-psychedelic 5-HT_{2A} receptor agonists (Halberstadt & Geyer, Psychopharmacol (Berl). 2013; 227(4):727-3).

Methods: An HTR assay is performed in accordance with the methods described in Klein et al., Neuropharmacol, 2018; 142:231-239 to assess the effects of disclosed compounds in mice. Male C57BL/6 J mice (6-8 weeks old) are obtained and housed in a vivarium that meets all requirements for care and treatment of laboratory animals. Mice are housed up to four per cage in a climate-controlled room on a reverse-light cycle (lights on at 1900 h, off at 0700 h) and are provided with ad libitum access to food and water, except during behavioral testing. Testing is conducted between 1000 and 1800 h. All animal experiments are conducted in accordance with applicable guidelines and are approved by an appropriate animal care committee.

A head-mounted magnet and a magnetometer detection coil is used to assess HTR, as previously described (Halberstadt & Geyer, Psychopharmacol (Berl). 2013; 227(4):727-3, Halberstadt & Geyer, Neuropharmacol, 2014; 77:200-7; Nichols et al., ACS Chem Neurosci. 2015; 6(7):1165-1175). Briefly, mice are anesthetized and a small neodymium magnet is attached to the dorsal cranial surface using dental cement. Following a 2-week recovery period, HTR experiments are carried out in a well-lit room with at least 7 days between sessions to avoid carryover effects.

Test compounds are dissolved in a suitable solvent, e.g., water containing 5% Tween 80, and administered IP at a volume of 5 or 10 mL/kg body weight immediately prior to testing. Different doses are tested to produce a dose-response curve. Mice are injected with drug or vehicle, and HTR activity is recorded in a glass cylinder surrounded by a magnetometer coil for 30 min. Coil voltage is low-pass filtered (2e10 kHz cutoff frequency), amplified, and digitized (20 kHz sampling rate) using a Powerlab/8SP with LabChart v 7.3.2 (ADInstruments, Colorado Springs, CO, USA), then filtered off-line (40e200 Hz band-pass).

Head twitches are identified manually based on the following criteria: 1) sinusoidal wavelets; 2) evidence of at least two sequential head movements (usually exhibited as bipolar peaks) with frequency 40 Hz; 3) amplitude exceeding the level of background noise; 4) duration <0.15 s; and 5) stable coil voltage immediately preceding and succeeding each response.

Head twitch counts are analyzed using one-way analyses of variance (ANOVA). Post hoc pairwise comparisons between selected groups are performed using Tukey's studentized range method. The entire recordings are examined for head twitches. In some cases a shorter block of time is analyzed to accommodate compounds with a brief duration-of-action, as potency calculations can be confounded by extended periods of inactivity. ED₅₀ values and 95% confidence limits are calculated using nonlinear regression. Relationships between HTR potency and binding affinities are assessed using linear regression and ordinary least-squares regression. For all analyses, significance is demonstrated by surpassing an α-level of 0.05.

Results can show that disclosed compounds are likely to produce psychedelic effects in humans. The magnitude of such effects is evaluated and compared among compounds. Results can be represented as ED₅₀ (mg/kg). Differences between the mouse HTR of disclosed compounds and suitable comparator compounds are also determined according to the methods described herein.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise compositions, formulations, methods, or the like disclosed; many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

1. A compound of Formula (1): 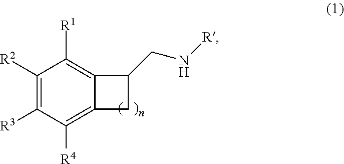
or a pharmaceutically acceptable salt, stereoisomer, hydrate, solvate, or isotopic derivative thereof, wherein:
R¹ is H or —OCH₃;
R² is H, or is taken together with R³ to form a methylenedioxy group;
R³ is H, Br, Cl, I, C₁-C₆ alkyl, C₁-C₆ alkylthio, or C₃-C₆ cycloalkylmethyl;
or R³ is taken together with R² to form a methylenedioxy group;
R⁴ is H, —OCH₃, or —CH₂CH₂OCH₃;
R′ is H or C₁-C₆ alkyl; and
n is 1 or 2; and
when R³ is H or Br, R⁴ is not —OCH₃; and
when R² and R³ are taken together to form a methylenedioxy group, and R¹ and R⁴ are both H, R′ is C₁-C₆ alkyl.

2. The compound of claim 1, wherein the compound has the structure of Formula (1A):

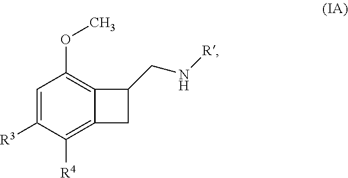

3. The compound of claim 2, wherein R⁴ is —OCH₃.

4. The compound of claim 2, wherein R⁴ is —CH₂CH₂OCH₃.

5. The compound of claim 2, wherein R′ is H.

6. The compound of claim 2, wherein R³ is I.

7. The compound of claim 2, wherein R³ is C₁-C₆ alkyl, isobutyl, or ethyl.

8-9. (canceled)

10. The compound of claim 2, wherein R³ is C₃-C₆ cycloalkylmethyl or cyclopropylmethyl.

11. (canceled)

12. The compound of claim 2, wherein the compound is the R-isomer.

13. The compound of claim 1, wherein the compound has the structure of Formula (1A-I):

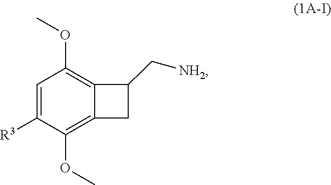

14. The compound of claim 13, wherein R³ is I.

15. The compound of claim 13, wherein R³ is C₁-C₆ alkyl.

16. The compound of claim 15, wherein R³ is isobutyl or ethyl.

17. (canceled)

18. The compound of claim 13, wherein the compound is the R-isomer.

19. The compound of claim 1, wherein the compound has the structure of Formula (1B):

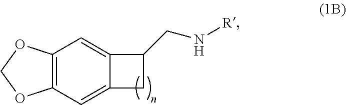

20-30. (canceled)

31. The compound of claim 1, having the structure of:

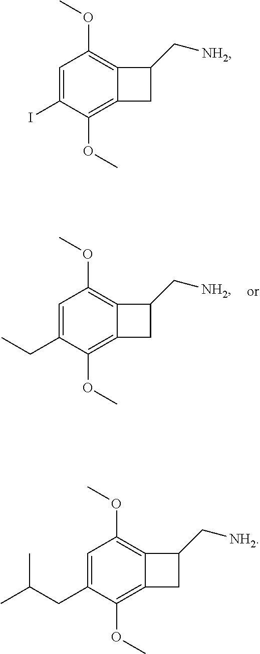 [SMILES: COc1cc(I)c(OC)c2c1C(CN)C2 ; CCc1cc(OC)c2c(c1OC)CC2CN ; COc1cc(CC(C)C)c(OC)c2c1C(CN)C2]

32. The compound of claim 1, having the structure of:

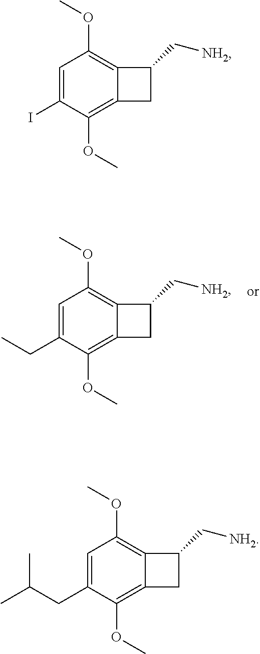 [SMILES: COc1cc(I)c(OC)c2c1[C@H](CN)C2 ; CCc1cc(OC)c2c(c1OC)C[C@H]2CN ; COc1cc(CC(C)C)c(OC)c2c1[C@H](CN)C2]

33-38. (canceled)

39. A pharmaceutical composition comprising a therapeutically effective amount of the compound of claim 1, or a pharmaceutically acceptable salt, stereoisomer, hydrate, solvate, or isotopic derivative thereof, and a pharmaceutically acceptable carrier, diluent, or excipient.

40. The pharmaceutical composition of claim 39, wherein the composition is suitable for oral, buccal, sublingual, intranasal, injectable, subcutaneous, intravenous, intraocular, topical, or transdermal administration.

41-49. (canceled)

50. A method of treating a medical condition in a subject in need of such treatment, the method comprising administering to the subject a therapeutically effective amount of the compound of claim 1, or a pharmaceutically acceptable salt, stereoisomer, hydrate, solvate, or isotopic derivative thereof, wherein the medical condition is a disorder linked to dysregulation or inadequate functioning of serotonergic neurotransmission; a mental, behavioral, or neurodevelopmental disorder; inflammation or an inflammatory disorder; an ophthalmic disorder; or a neurodegenerative disorder.

51-112. (canceled)
